(19) United States
(12) Patent Application Publication
(10) Pub. No.: US 2010/0280545 A1
(43) Pub. Date: Nov. 4, 2010

(54) SYSTEM AND METHOD FOR BONDING LIVING TISSUE

(75) Inventor: Ofer Fridman, D.N. Bikat Beit Hakerem (IL)

Correspondence Address: FISH & RICHARDSON P.C., P.O. BOX 1022, MINNEAPOLIS, MN, 55440-1022, US

(73) Assignee: SERAFFIX LTD., D.N.. MISGAVE (IL)

(21) Appl. No.: 12/599,571

(22) PCT Filed: Apr. 30, 2008
(86) PCT No.: PCT/IL08/00580

§ 371 Date: Nov. 10, 2009

Related U.S. Application Data
(60) Provisional application No. 60/917,085, filed on May 10, 2007.

(51) Int. Cl. A61B 17/00

(52) U.S. Cl. 606/213

(57) ABSTRACT

A system for bonding living tissue including first tissue displacer functionality operative initially to cause displacement of edges of tissue into a mutual touching relationship along a seam prior to bonding thereof and second tissue displacer functionality operative following operation of the first tissue displacer functionality to cause displacement of the edges into a mutual non-touching relationship wherein the edges are in mutual propinquity.

### REFERENCE TO RELATED APPLICATIONS

Reference is made to the following related patent applications, the contents of which are hereby incorporated by reference: U.S. Provisional Patent Application Ser. No. 60/863,818, filed Nov. 1, 2006, entitled NOVEL LASER SOLDERING SYSTEM HAND PIECE; U.S. Provisional Patent Application Ser. No. 60/917,086, filed May 10, 2007, entitled METHOD AND SYSTEM FOR WOUND TREATING; PCT Patent Application PCT/IL2007/001338, filed Nov. 1, 2007; and PCT Patent Application PCT/IL2007/001339, filed Nov. 1, 2007.

Priority is hereby claimed under 37 CFR 1.78(a)(1), (2)i, (4) and (5)i from: U.S. Provisional Patent Application Ser. No. 60/917,086, filed May 10, 2007, entitled METHOD AND SYSTEM FOR WOUND TREATING; PCT Patent Application PCT/IL2007/001338, filed Nov. 1, 2007, and PCT Patent Application PCT/IL2007/001339, filed Nov. 1, 2007.

### FIELD OF THE INVENTION

The present invention relates to systems and methods for bonding living tissue.

### BACKGROUND OF THE INVENTION

The following U.S. Patent publications are believed to represent the current state of the art:

U.S. Pat. Nos. 6,211,335; 5,505,726 5,334,191; and 5,300,065.

### SUMMARY OF THE INVENTION

The present invention seeks to provide improved systems and methods for bonding living tissue.

There is thus provided in accordance with a preferred embodiment of the present invention a system for bonding living tissue including first tissue displacer functionality operative initially to cause displacement of edges of tissue into a mutual touching relationship along a seam prior to bonding thereof and second tissue displacer functionality operative following operation of the first tissue displacer functionality to cause displacement of the edges into a mutual non-touching relationship wherein the edges are in mutual propinquity.

Preferably, the system for bonding living tissue also includes tissue bonding functionality operative following operation of the first and second displacer functionalities to apply energy to the edges of the tissue and third tissue displacer functionality operative to cause displacement of the edges of the tissue into the mutual touching relationship along the seam for bonding thereof. Additionally, the system for bonding living tissue also includes a tissue bonding enhancement material dispenser operative following operation of the second tissue displacer functionality and at least partially prior to the tissue bonding functionality to apply a tissue bonding enhancement material to the edges when the edges are in the mutual non-touching relationship wherein the edges are in mutual propinquity.

In accordance with a preferred embodiment of the present invention the first and second tissue displacer functionalities employ a tissue displacer assembly including at least first and second tissue engagement elements arranged for removable engagement with tissue adjacent opposite facing edges thereof and relative displacement thereof, thereby to bring the opposite facing edges into mutual propinquity, at least one of the at least first and second tissue engagement elements being transparent to radiant energy at least at a region thereof overlying the opposite facing edges.

Preferably, the first and second tissue displacer functionalities employ a tissue displacer assembly including at least first and second tissue engagement elements arranged for removable engagement with tissue adjacent opposite facing edges thereof and at least first and second linearly displaceable elements operatively engageable with the first and second tissue engagement elements for providing relative displacement of the at least first and second tissue engagement elements, wherein operative engagement of the at least first and second linearly displaceable elements with the at least first and second tissue engagement elements includes at least the following modes of operation: a first closing mode of operation wherein generally parallel displacement of the first and second linearly displaceable elements towards each other produces generally non-parallel displacement of the at least first and second tissue engagement elements towards each other, a second opening mode of operation wherein generally parallel displacement of the first and second linearly displaceable elements away from each other produces generally parallel displacement of the at least first and second tissue engagement elements away from each other; and a third closing mode of operation wherein linear displacement of the first and second linearly displaceable elements towards each other produces generally parallel displacement of the at least first and second tissue engagement elements towards each other.

In accordance with a preferred embodiment of the present invention the first and second tissue displacer functionalities employ a tissue displacer assembly including at least first and second tissue engagement elements arranged for removable engagement with tissue adjacent opposite facing edges thereof for providing relative displacement of the opposite facing edges into and out of mutual propinquity. Additionally or alternatively, the first and second tissue displacer functionalities employ a tissue displacer assembly including at least first and second tissue engagement elements arranged for removable engagement with tissue adjacent opposite facing edges thereof and relative displacement thereof, thereby to bring the opposite facing edges into desired mutual propinquity and a bonding enhancement material dispenser mounted with respect to at least one of the at least first and second tissue engagement elements for dispensing tissue bonding enhancement material onto the opposite facing edges when in the desired mutual propinquity. Alternatively or additionally, the first and second tissue displacer functionalities employ a tissue displacer assembly including at least first and second tissue engagement elements arranged for removable engagement with tissue adjacent opposite facing edges thereof, at least first and second displaceable elements operatively engageable with the first and second tissue engagement elements for providing relative displacement of the at least first and second tissue engagement elements into and out of propinquity, such that the opposite facing edges of the tissue are mutually displaced into and out of touching engagement and at least one retainer operative, independently of the first and second displaceable elements, for retaining the at least first and second tissue engagement elements in propinquity, such that the opposite facing edges of the tissue are in touching engagement.

Preferably, the tissue bonding enhancement material dispenser is at least partially integrated with the tissue displacer assembly. Alternatively or additionally, the tissue bonding enhancement material dispenser includes a gas driven sprayer for applying the tissue bonding enhancement material onto the edges.

There is also provided in accordance with another preferred embodiment of the present invention a system for bonding living tissue including tissue bonding functionality operative to apply energy to edges of tissue and to cause displacement of the edges of the tissue into mutual touching relationship along a seam for bonding thereof.

Preferably, the system for bonding living tissue also includes a tissue bonding enhancement material dispenser operative at least partially prior to operation of the tissue bonding functionality to apply a tissue bonding enhancement material to the edges when the edges are in the mutual non-touching relationship wherein the edges are in mutual propinquity.

In accordance with a preferred embodiment of the present invention, the system for bonding living tissue also includes tissue displacer functionality operative prior to operation of the tissue bonding functionality to cause displacement of the edges into a mutual non-touching relationship wherein the edges are in mutual propinquity. Additionally or alternatively, the system for bonding living tissue also includes first tissue displacer functionality operative initially to cause displacement of edges of tissue into a mutual touching relationship along a seam prior to bonding thereof and second tissue displacer functionality operative following operation of the first tissue displacer functionality to cause displacement of the edges into a mutual non-touching relationship wherein the edges are in mutual propinquity.

Preferably, the tissue bonding functionality is operative initially to apply energy to relatively more interior portions of the edges of the tissue and to cause displacement of the relatively more interior portions of the edges of the tissue into the mutual touching relationship along the seam for bonding thereof and thereafter to apply energy to relatively less interior portions of the edges of the tissue and to cause displacement of the relatively less interior portions of the edges of the tissue into the mutual touching relationship along the seam for bonding thereof. Additionally, the tissue bonding functionality is operative sequentially initially to apply energy to relatively more interior portions of the edges of the tissue and to cause displacement of the relatively more interior portions of the edges of the tissue into the mutual touching relationship along the seam for bonding thereof and thereafter sequentially to apply energy to increasingly less interior portions of the edges of the tissue and to cause displacement of the increasingly less interior portions of the edges of the tissue into the mutual touching relationship along the seam for bonding thereof.

In accordance with a preferred embodiment of the present invention the tissue displacer functionality employs a tissue displacer assembly including at least first and second tissue engagement elements arranged for removable engagement with tissue adjacent opposite facing edges thereof and relative displacement thereof, thereby to bring the opposite facing edges into mutual propinquity, at least one of the at least first and second tissue engagement elements being transparent to radiant energy at least at a region thereof overlying the opposite facing edges.

Preferably, the first and second tissue displacer functionalities employ a tissue displacer assembly including at least first and second tissue engagement elements arranged for removable engagement with tissue adjacent opposite facing edges thereof and at least first and second linearly displaceable elements operatively engageable with the first and second tissue engagement elements for providing relative displacement of the at least first and second tissue engagement elements, wherein operative engagement of the at least first and second linearly displaceable elements with the at least first and second tissue engagement elements includes at least the following modes of operation: a first closing mode of operation wherein generally parallel displacement of the first and second linearly displaceable elements towards each other produces generally non-parallel displacement of the at least first and second tissue engagement elements towards each other, a second opening mode of operation wherein generally parallel displacement of the first and second linearly displaceable elements away from each other produces generally parallel displacement of the at least first and second tissue engagement elements away from each other and a third closing mode of operation wherein linear displacement of the first and second linearly displaceable elements towards each other produces generally parallel displacement of the at least first and second tissue engagement elements towards each other.

In accordance with a preferred embodiment of the present invention the first and second tissue displacer functionalities employ a tissue displacer assembly including at least first and second tissue engagement elements arranged for removable engagement with tissue adjacent opposite facing edges thereof for providing relative displacement of the opposite facing edges into and out of mutual propinquity. Additionally or alternatively, the first and second tissue displacer functionalities employ a tissue displacer assembly including at least first and second tissue engagement elements arranged for removable engagement with tissue adjacent edges thereof and relative displacement thereof, thereby to bring the edges into desired mutual propinquity and a bonding enhancement material dispenser mounted with respect to at least one of the at least first and second tissue engagement elements for dispensing tissue bonding enhancement material onto the edges when in the desired mutual propinquity.

There is further provided in accordance with yet another preferred embodiment of the present invention a system for bonding living tissue including tissue bonding functionality operative to apply energy to edges of tissue for bonding thereof along a seam and including a computer-controlled energy impingement location displacer for displacement of an impingement location of the energy along the seam.

Preferably, the computer-controlled energy impingement location displacer includes a displacer that is capable of movement in at least two mutually perpendicular directions in a plane. Alternatively, the computer-controlled energy impingement location displacer includes a displacer that is capable of movement in at least three mutually perpendicular directions.

In accordance with a preferred embodiment of the present invention the computer-controlled energy impingement location displacer includes a seam designator operative to designate the seam in a frame of reference and is operative to displace the impingement location of the energy along the seam, designated by the seam designator. Additionally, the computer-controlled energy impingement location displacer includes a user interface enabling an operator employing the user interface to designate the seam.

Preferably, the tissue bonding functionality includes a laser. Additionally, the tissue bonding functionality also includes an optical waveguide carrying energy from the laser and the computer-controlled energy impingement location displacer displaces at least an end of the optical waveguide.

In accordance with a preferred embodiment of the present invention the system for bonding living tissue also includes a computer-controlled tissue displacer operative to cause displacement of the edges of the tissue into mutual touching relationship along the seam for bonding thereof in coordination with operation of the computer-controlled energy impingement location displacer, whereby application of energy to the edges of the tissue at a given location is quickly followed by displacement of the edges of the tissue at the location into mutual touching relationship. Additionally, the operation of the computer-controlled tissue displacer and of the computer-controlled energy impingement location displacer are coordinated such that application of energy to the edges of the tissue at a given location followed by displacement of the edges of the tissue at the location into mutual touching relationship proceeds sequentially along the seam.

Preferably, the computer-controlled energy impingement location displacer includes a displaceable optical pathway and the system for bonding living tissue also includes a temperature sensor operative to sense temperature at the energy impingement location along the seam via at least part of the displaceable optical pathway.

In accordance with a preferred embodiment of the present invention the computer-controlled tissue displacer includes a tissue tension force sensor for measuring the amount of tensile force applied to the edges of the tissue when displaced into the mutually touching relationship. Additionally, the tissue tension force sensor is operative to provide an output indication of a measured tissue tension parameter.

Preferably, the system for bonding living tissue also includes first tissue displacer functionality operative initially to cause displacement of edges of tissue into a mutual touching relationship along a seam prior to bonding thereof and second tissue displacer functionality operative following operation of the first tissue displacer functionality to cause displacement of the edges into a mutual non-touching relationship wherein the edges are in mutual propinquity. Additionally, the first and second tissue displacer functionalities employ a tissue displacer assembly including at least first and second tissue engagement elements arranged for removable engagement with tissue adjacent opposite facing edges thereof and relative displacement thereof, thereby to bring the opposite facing edges into mutual propinquity, at least one of the at least first and second tissue engagement elements being transparent to radiant energy at least at a region thereof overlying the opposite facing edges. Alternatively, the first and second tissue displacer functionalities employ a tissue displacer assembly including at least first and second tissue engagement elements arranged for removable engagement with tissue adjacent opposite facing edges thereof and at least first and second linearly displaceable elements operatively engageable with the first and second tissue engagement elements for providing relative displacement of the at least first and second tissue engagement elements, wherein operative engagement of the at least first and second linearly displaceable elements with the at least first and second tissue engagement elements includes at least the following modes of operation: a first closing mode of operation wherein generally parallel displacement of the first and second linearly displaceable elements towards each other produces generally non-parallel displacement of the at least first and second tissue engagement elements towards each other, a second opening mode of operation wherein generally parallel displacement of the first and second linearly displaceable elements away from each other produces generally parallel displacement of the at least first and second tissue engagement elements away from each other and a third closing mode of operation wherein linear displacement of the first and second linearly displaceable elements towards each other produces generally parallel displacement of the at least first and second tissue engagement elements towards each other.

Preferably, the first and second tissue displacer functionalities employ a tissue displacer assembly including at least first and second tissue engagement elements arranged for removable engagement with tissue adjacent opposite facing edges thereof for providing relative displacement of the opposite facing edges into and out of mutual propinquity. Alternatively, the first and second tissue displacer functionalities employ a tissue displacer assembly including at least first and second tissue engagement elements arranged for removable engagement with tissue adjacent opposite facing edges thereof and relative displacement thereof, thereby to bring the edges into desired mutual propinquity and a bonding enhancement material dispenser mounted with respect to at least one of the at least first and second tissue engagement elements for dispensing tissue bonding enhancement material onto the edges when in the desired mutual propinquity.

In accordance with a preferred embodiment of the present invention the first and second tissue displacer functionalities employ a tissue displacer assembly including at least first and second tissue engagement elements arranged for removable engagement with tissue adjacent opposite facing edges thereof, at least first and second displaceable elements operatively engageable with the first and second tissue engagement elements for providing relative displacement of the at least first and second tissue engagement elements into and out of propinquity, such that the opposite facing edges of the tissue are mutually displaced into and out of touching engagement and at least one retainer operative, independently of the first and second displaceable elements, for retaining the at least first and second tissue engagement elements in propinquity, such that the opposite facing edges of the tissue are in touching engagement.

Preferably, the system for bonding living tissue also includes a tissue bonding enhancement material dispenser. Additionally, the tissue bonding enhancement material dispenser includes a gas driven sprayer for applying the tissue bonding enhancement material onto the edges.

There is even further provided in accordance with still another preferred embodiment of the present invention a tissue displacer assembly including at least first and second tissue engagement elements arranged for removable engagement with tissue adjacent opposite facing edges thereof and at least first and second linearly displaceable elements operatively engageable with the first and second tissue engagement elements for providing relative displacement of the at least first and second tissue engagement elements, wherein operative engagement of the at least first and second linearly displaceable elements with the at least first and second tissue engagement elements includes at least the following modes of operation a first closing mode of operation wherein generally parallel displacement of the first and second linearly displaceable elements towards each other produces generally non-parallel displacement of the at least first and second tissue engagement elements towards each other, a second opening mode of operation wherein generally parallel displacement of the first and second linearly displaceable elements away from each other produces generally parallel displacement of the at least first and second tissue engagement elements away from each other and a third closing mode of operation wherein linear displacement of the first and second linearly displaceable elements towards each other produces generally parallel displacement of the at least first and second tissue engagement elements towards each other.

Preferably, at least one of the at least first and second tissue engagement elements is transparent to radiant energy at least at a region thereof overlying the opposite facing edges. Additionally or alternatively, the tissue displacer assembly also includes at least one retainer operative, independently of the first and second displaceable elements, for retaining the at least first and second tissue engagement elements in propinquity, such that the opposite facing edges of the tissue are in touching engagement.

There is yet further provided in accordance with another preferred embodiment of the present invention a tissue manipulation assembly including a chassis; a plurality of individually controllable motors; a plurality of arms each at least partially positioned by one of the plurality of motors and a tissue engagement head mounted onto each of the plurality of arms, the tissue manipulation assembly having at least the following modes of operation: a first closing mode of operation wherein generally non-parallel displacement of the first and second tissue engagement heads towards each other takes place and a second closing mode of operation wherein linear displacement of the first and second tissue engagement heads towards each other produces generally parallel displacement of the at least first and second tissue engagement elements towards each other.

Preferably, the first closing mode of operation initially employs less than all of the tissue engagement heads. Additionally or alternatively, the tissue engagement head includes a vacuum engagement head arranged for selectable engagement with tissue adjacent a cut.

There is still further provided in accordance with yet another preferred embodiment of the present invention a tissue displacer assembly including at least first and second tissue engagement elements arranged for removable engagement with tissue adjacent opposite facing edges thereof and relative displacement thereof, thereby to bring the opposite facing edges into mutual propinquity, at least one of the at least first and second tissue engagement elements being transparent to radiant energy at least at a region thereof overlying the opposite facing edges.

Preferably, the tissue displacer assembly also includes at least one retainer operative, independently of the first and second displaceable elements, for retaining the at least first and second tissue engagement elements in propinquity, such that the opposite facing edges of the tissue are in touching engagement.

There is also further provided in accordance with still another preferred embodiment of the present invention a tissue displacer assembly including at least first and second tissue engagement elements arranged for removable engagement with tissue adjacent opposite facing edges thereof for providing relative displacement of the opposite facing edges into and out of mutual propinquity.

Preferably, relative displacement of the at least first and second tissue engagement elements includes at least the following modes of operation: a first closing mode of operation wherein generally parallel displacement of the first and second tissue engagement elements towards each other brings the opposite facing edges towards each other and a second opening mode of operation wherein generally parallel displacement of the first and second tissue engagement elements away from each other moves the opposite facing edges away from each other. Additionally or alternatively the tissue displacer assembly also includes at least one retainer operative, independently of the first and second displaceable elements, for retaining the at least first and second tissue engagement elements in propinquity, such that the opposite facing edges of the tissue are in touching engagement. Additionally, the at least one retainer includes a bandage assembly including a pair of side bandage portions, each having an adhesive surface for attachment to a patient's body and a release layer, a plurality of conduits, fixed to each bandage portion and a plurality of elongate elements each extending through a corresponding one of the plurality of conduits, one end of each of the elongate elements extending through conduits on each side bandage portion being connected to a common pull-tab and an opposite end of each of the elongate elements extending through conduits on each side bandage portion being fixed to an opposite side bandage portion.

There is further provided in accordance with even another preferred embodiment of the present invention a tissue displacer assembly including at least first and second tissue engagement elements arranged for removable engagement with tissue adjacent opposite facing edges thereof and relative displacement thereof, thereby to bring the edges into desired mutual propinquity and a bonding enhancement material dispenser mounted with respect to at least one of the at least first and second tissue engagement elements for dispensing tissue bonding enhancement material onto the edges when in the desired mutual propinquity.

Preferably, the bonding enhancement material dispenser includes a gas driven sprayer for applying the tissue bonding enhancement material onto the edges.

In accordance with a preferred embodiment of the present invention the tissue displacer assembly also includes at least one retainer operative, independently of the first and second displaceable elements, for retaining the at least first and second tissue engagement elements in propinquity, such that the edges of the tissue are in touching engagement. Additionally, the at least one retainer includes a bandage assembly including a pair of side bandage portions, each having an adhesive surface for attachment to a patient's body and a release layer, a plurality of conduits, fixed to each bandage portion and a plurality of elongate elements each extending through a corresponding one of the plurality of conduits, one end of each of the elongate elements extending through conduits on each side bandage portion being connected to a common pull-tab and an opposite end of each of the elongate elements extending through conduits on each side bandage portion being fixed to an opposite side bandage portion.

There is still further provided in accordance with still another preferred embodiment of the present invention a bandage assembly including a pair of side bandage portions, each having an adhesive surface for attachment to a patient's body and a release layer, a plurality of conduits, fixed to each bandage portion and a plurality of elongate elements each extending through a corresponding one of the plurality of conduits, one end of each of the elongate elements extending through conduits on each side bandage portion being connected to a common pull-tab and an opposite end of each of the elongate elements extending through conduits on each side bandage portion being fixed to an opposite side bandage portion.

There is yet further provided in accordance with yet another preferred embodiment of the present invention a tissue displacer assembly including at least first and second tissue engagement elements arranged for removable engagement with tissue adjacent opposite facing edges thereof, at least first and second displaceable elements operatively engageable with the first and second tissue engagement elements for providing relative displacement of the at least first and second tissue engagement elements into and out of propinquity, such that the opposite facing edges of the tissue are mutually displaced into and out of touching engagement and at least one retainer operative, independently of the first and second displaceable elements, for retaining the at least first and second tissue engagement elements in propinquity, such that the opposite facing edges of the tissue are in touching engagement.

Preferably, the at least one retainer includes a bandage assembly including a pair of side bandage portions, each having an adhesive surface for attachment to a patient's body and a release layer, a plurality of conduits, fixed to each bandage portion and a plurality of elongate elements each extending through a corresponding one of the plurality of conduits, one end of each of the elongate elements extending through conduits on each side bandage portion being connected to a common pull-tab and an opposite end of each of the elongate elements extending through conduits on each side bandage portion being fixed to an opposite side bandage portion.

There is also provided in accordance with still another preferred embodiment of the present invention a system for bonding living tissue including a radiant energy tissue bonder operative to apply energy to adjacent edges of the tissue for bonding thereof and a post energy application tissue displacer being operative to be displaced along a seam defined by the adjacent edges of the tissue and to cause displacement of the edges of tissue into a mutual touching relationship along the seam subsequent to application of energy thereto.

Preferably, the system for bonding living tissue also includes a hand-held element supporting the radiant energy tissue bonder and the first tissue displacer. Additionally or alternatively, the system for bonding living tissue also includes a pre-energy application tissue displacer being operative to be displaced along a seam defined by the adjacent edges and to cause displacement of the edges of tissue into desired mutual propinquity along the seam prior to application of energy thereto.

In accordance with a preferred embodiment of the present invention the system for bonding living tissue also includes a dispenser for a tissue bonding enhancement material which is operative to dispense the tissue bonding enhancement material onto the edges of the tissue prior to application of energy thereto. Additionally or alternatively, the system for bonding living tissue also includes an energy application controller operative to govern the application of energy to the edges of the tissue.

Preferably, the system for bonding living tissue also includes a displacement controller operative to govern the displacement of the post energy application tissue displacer along the seam. Additionally or alternatively, the system for bonding living tissue also includes a dispenser controller for controlling the rate of dispensing of the tissue bonding enhancement material onto the edges of the tissue.

In accordance with a preferred embodiment of the present invention the tissue displacer employs a tissue displacer assembly including at least first and second tissue engagement elements arranged for removable engagement with tissue adjacent opposite facing edges thereof and relative displacement thereof, thereby to bring the opposite facing edges into mutual propinquity, at least one of the at least first and second tissue engagement elements being transparent to radiant energy at least at a region thereof overlying the opposite facing edges. Alternatively or additionally, the first and second tissue displacer functionalities employ a tissue displacer assembly including at least first and second tissue engagement elements arranged for removable engagement with tissue adjacent opposite facing edges thereof and at least first and second linearly displaceable elements operatively engageable with the first and second tissue engagement elements for providing relative displacement of the at least first and second tissue engagement elements, wherein operative engagement of the at least first and second linearly displaceable elements with the at least first and second tissue engagement elements includes at least the following modes of operation: a first closing mode of operation wherein generally parallel displacement of the first and second linearly displaceable elements towards each other produces generally non-parallel displacement of the at least first and second tissue engagement elements towards each other, a second opening mode of operation wherein generally parallel displacement of the first and second linearly displaceable elements away from each other produces generally parallel displacement of the at least first and second tissue engagement elements away from each other and a third closing mode of operation wherein linear displacement of the first and second linearly displaceable elements towards each other produces generally parallel displacement of the at least first and second tissue engagement elements towards each other.

Preferably, the first and second tissue displacer functionalities employ a tissue displacer assembly including at least first and second tissue engagement elements arranged for removable engagement with tissue adjacent opposite facing edges thereof for providing relative displacement of the opposite facing edges into and out of mutual propinquity. Additionally or alternatively, the first and second tissue displacer functionalities employ a tissue displacer assembly including at least first and second tissue engagement elements arranged for removable engagement with tissue adjacent opposite facing edges thereof and relative displacement thereof, thereby to bring the edges into desired mutual propinquity and a bonding enhancement material dispenser mounted with respect to at least one of the at least first and second tissue engagement elements for dispensing tissue bonding enhancement material onto the edges when in the desired mutual propinquity.

In accordance with a preferred embodiment of the present invention the first and second tissue displacer functionalities employ a tissue displacer assembly including at least first and second tissue engagement elements arranged for removable engagement with tissue adjacent opposite facing edges thereof, at least first and second displaceable elements operatively engageable with the first and second tissue engagement elements for providing relative displacement of the at least first and second tissue engagement elements into and out of propinquity, such that the opposite facing edges of the tissue are mutually displaced into and out of touching engagement and at least one retainer operative, independently of the first and second displaceable elements, for retaining the at least first and second tissue engagement elements in propinquity, such that the opposite facing edges of the tissue are in touching engagement.

### BRIEF DESCRIPTION OF THE DRAWINGS

The present invention will be understood and appreciated more fully from the following detailed description, taken in conjunction with the drawings in which:

FIGS. 1A and 1B are together a simplified illustration of the operation of a system and method for bonding living tissue in accordance with a preferred embodiment of the invention;

FIGS. 2A and 2B are together a simplified illustration of the operation of a system and method for bonding living tissue in accordance with another preferred embodiment of the present invention;

FIG. 3 is a simplified pictorial view illustration of the system of FIG. 1A constructed and operative in accordance with one embodiment of the present invention in an operative orientation on a patient;

FIGS. 4A, 4B, 4C and 4D are simplified pictorial and plan view illustrations of a patient-mounted portion of the system of FIG. 3, FIG. 4B being an underside plan view taken along arrow B in FIG. 4A, FIGS. 4C and 4D being respective end and side views taken along respective arrows C and D in FIG. 4A;

FIG. 5 is a simplified partially exploded view illustration of the patient-mounted portion of the system of FIG. 3 in accordance with one embodiment of the present invention;

FIG. 6 is a simplified partially exploded view illustration of a clamp assembly forming part of the system of FIG. 3;

FIGS. 7A and 7B are each a simplified exploded view illustration of a clamp portion of the clamp assembly of FIG. 6 in accordance with two alternative embodiments of the present invention;

FIGS. 8A, 8B, 8C and 8D are each a simplified illustration of a first part of the clamp portion of FIG. 7A;

FIGS. 9A, 9B, 9C, 9D and 9E are each a simplified illustration of a second part of the clamp portion of FIG. 7A;

FIGS. 10A, 10B, 10C and 10D are each a simplified illustration of a third part of the clamp portion of FIG. 7A;

FIGS. 11A, 11B, 11C, 11D, 11E and 11F are each a simplified illustration of a fourth part of the clamp portion of FIG. 7A;

FIGS. 12A, 12B, 12C, 12D and 12E are each a simplified illustration of a fifth part of the clamp portion of FIG. 7A;

FIGS. 13A, 13B, 13C, 13D and 13E are each a simplified illustration of a sixth part of the clamp portion of FIG. 7A;

FIGS. 14A, 14B, 14C, 14D and 14E are each a simplified illustration of a seventh part of the clamp portion of FIG. 7A;

FIGS. 15A, 15B, 15C, 15D and 15E are each a simplified illustration of a eighth part of the clamp portion of FIG. 7A;

FIGS. 16A, 16B, 16C, 16D and 16E are each a simplified illustration of a ninth part of the clamp portion of FIG. 7A;

FIGS. 17A, 17B, 17C, 17D, 17E and 17F are each a simplified illustration of a third part of the clamp portion of FIG. 7B;

FIGS. 18A, 18B, 18C, 18D, 18E and 18F are each a simplified illustration of a fourth part of the clamp portion of FIG. 7B;

FIGS. 19A, 19B, 19C, 19D, 19E and 19F are each a simplified illustration of a fifth part of the clamp portion of FIG. 7B;

FIGS. 20A and 20B are each a simplified pictorial illustration of a clamp engagement element forming part of the system of FIGS. 4A-4D and 5;

FIGS. 21A, 21B, 21C and 21D are each a simplified illustration of a clamp manipulation subassembly forming part of the system of FIGS. 4A-4D and 5;

FIGS. 22A and 22B are each a simplified illustration of a tissue manipulation subassembly forming part of an alternative embodiment of the system of FIGS. 4A-4D and 5;

FIG. 23 is a simplified partially exploded view illustration of a bandage assembly forming part of an alternative embodiment of the system of FIG. 3 and useful with the tissue manipulation subassembly of FIGS. 22A and 22B;

FIGS. 24A and 24B are each a simplified pictorial illustration of a tissue bonding enhancement material dispenser subassembly forming part of the system of FIGS. 4A-4D and 5;

FIGS. 25A and 25B are each a simplified pictorial illustration of an alternative embodiment of a tissue bonding enhancement material dispenser subassembly forming part of the system of FIGS. 4A-4D and 5;

FIGS. 26A, 26B and 26C are each a simplified pictorial illustration of an energy directing subassembly forming part of the system of FIGS. 4A-4D;

FIGS. 27A, 27B and 27C are each a simplified pictorial illustration of a stage in the operation of the system of FIGS. 1A, 1B and 3-21D and 24A-26C, wherein the clamp assembly (FIG. 6) is mounted onto a patient in propinquity to a cut to be bonded;

FIGS. 28A, 28B, 28C, 28D and 28E are each a simplified pictorial illustration of another stage in the operation of the system of FIGS. 1A, 1B and 3-21D and 24A-26C, wherein clamp engagement elements (FIGS. 20A & 20B) are retained in the clamp manipulation subassembly (FIGS. 21A-21D);

FIGS. 29A, 29B and 29C are each a simplified pictorial illustration of further stage in the operation of the system of FIGS. 1A, 1B and 3-21D and 24A-26C, wherein clamp engagement elements (FIGS. 20A & 20B), while retained in the clamp manipulation subassembly (FIGS. 21A-21D), are caused to engage the clamp assembly and produce initial closing displacement thereof;

FIGS. 30A and 30B are each a simplified pictorial illustration of a yet further stage in the operation of the system of FIGS. 1A, 1B, 3-21D and 24A-26C, wherein clamp engagement elements (FIGS. 20A & 20B) are displaced by the clamp manipulation subassembly (FIGS. 21A-21D) in engagement with the clamp assembly prior to application of energy or of bonding enhancement material;

FIGS. 31A and 31B are each a simplified pictorial illustration of a still further stage in the operation of the system of FIGS. 1A, 1B and 3-7A and 8A-16E and 20A-24B and 26A-26C, wherein bonding enhancement material is applied in accordance with one embodiment of the present invention employing an air brush applicator;

FIG. 32 is a simplified pictorial illustration of a bonding enhancement material application stage in the operation of the system of FIGS. 1A, 1B and 3-6 and 7B-9E and 13A-21D and 26A-26C, wherein bonding enhancement material is applied in accordance with yet another embodiment of the present invention employing clamp mounted applicators;

FIGS. 33A, 33B and 33C are each a simplified pictorial illustration of a bonding enhancement material application stage in the operation of the system of FIGS. 1A, 1B and 3-7A and 8A-16E and 20A-23 and 25A-26C, wherein bonding enhancement material is applied in accordance with still another embodiment of the present invention, wherein a tissue bonding enhancement material dispenser subassembly comprises a material supply assembly including a container which is coupled via a valve to a source of pressurized gas (not shown);

FIGS. 34A, 34B, 34C, 34D, 34E, 34F and 34G are each a simplified pictorial illustration of a still further stage in the operation of the system of FIGS. 1A, 1B and 3-26C, wherein energy is applied to the cut by the energy directing subassembly (FIGS. 26A-26C) in coordination with operation of the clamp manipulation subassembly (FIGS. 21A-21D) or the tissue manipulation subassembly (FIGS. 22A and 22B);

FIGS. 35A, 35B and 35C are each a simplified pictorial illustration of an additional stage in the operation of the system of FIGS. 1A, 1B and 3-26C, wherein an operator secures the cut in a bonded orientation by use of the clamp assembly (FIG. 6);

FIGS. 36A, 36B and 36C illustrate a further additional stage in the operation of the system of FIGS. 1A, 1B and 3-21D and 24A-26C wherein the clamp engagement elements 252 and 254 (FIGS. 20A and 20B) are released from the clamp manipulation subassembly 256 (FIGS. 21A-21D) and disengaged from the clamp assembly;

FIGS. 37A and 37B illustrate a final stage in the operation of the system of FIGS. 1A, 1B, 3-21D and 24A-26C wherein the clamp portion is separated from the remainder of the clamp assembly and an auxiliary bandage is placed over the cut and the remainder of the clamp assembly;

FIGS. 38A, 38B and 38C are each a simplified pictorial illustration of a stage in the operation of the system of FIGS. 1A, 1B and 3-5 and 22A-26C, wherein the bandage assembly (FIG. 23) is mounted onto a patient in propinquity to a cut to be bonded;

FIGS. 39A, 39B and 39C are each a simplified pictorial illustration of a further stage in the operation of the system of FIGS. 1A, 1B and 3-5 and 22A-26C wherein the tissue manipulation subassembly (FIGS. 22A & 22B) is employed to provide initial closing of a cut to be bonded;

FIG. 40 is a simplified pictorial illustration of a further stage in the operation of the system of FIGS. 1A, 1B and 3-5 and 22A-26C wherein the tissue manipulation subassembly (FIGS. 22A & 22B) is used to provide opening displacement of the cut prior to the application of energy or of bonding enhancement material;

FIG. 41 is a simplified illustration of a further additional stage in the operation of the system of FIGS. 1A, 1B and 3-5 and 22A-26C, wherein the tissue manipulation subassembly (FIGS. 22A & 22B) is disengaged from the patient; and

FIG. 42 illustrates a final stage in the operation of the system of FIGS. 1A, 1B and 3-5 and 22A-26C, wherein an auxiliary bandage is placed over the cut and the bandage assembly (FIG. 23).

### DETAILED DESCRIPTION OF PREFERRED EMBODIMENTS

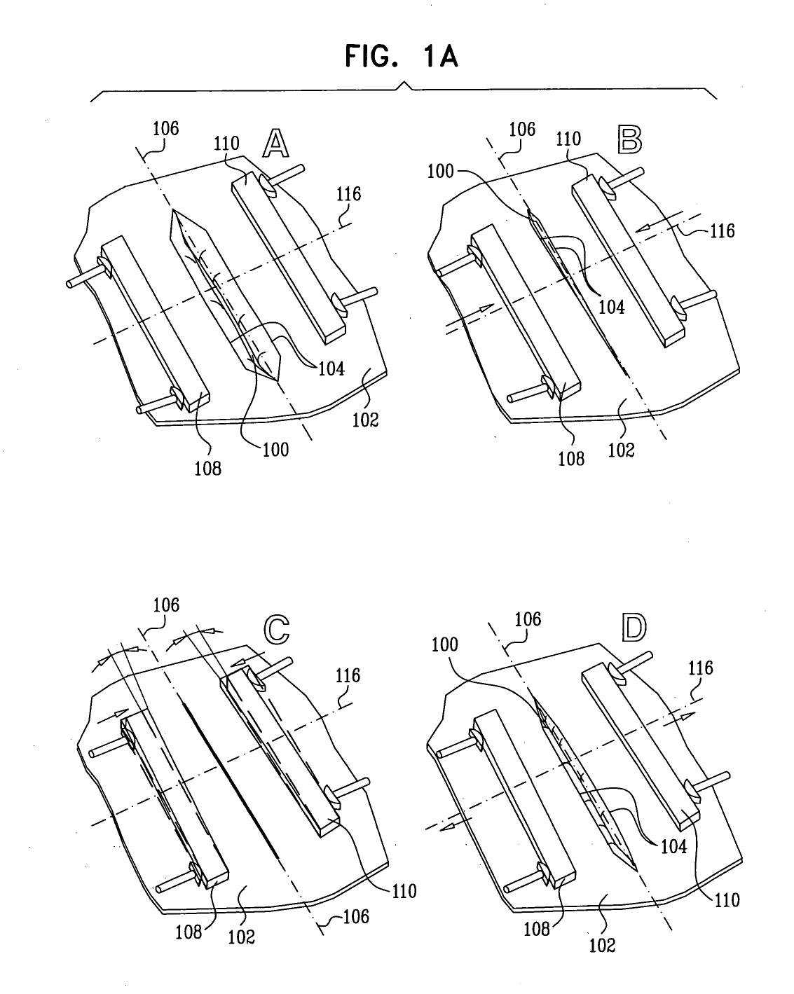

Reference is now made to FIGS. 1A and 1B, which are, together, a simplified illustration of the operation of a system and method for bonding living tissue in accordance with a preferred embodiment of the invention. FIGS. 1A and 1B show a cut 100 in living tissue 102 which defines edges 104 of the tissue 102. The term “cut” refers to any separation of edges of tissue which it is sought to bond and can include incisions, excisions and wounds. In accordance with the present invention there is provided first tissue displacer functionality operative initially to cause displacement of edges 104 of tissue 102 into a mutual touching relationship along a seam, here shown extending along a longitudinal axis 106, prior to bonding thereof; second tissue displacer functionality operative following operation of the first tissue displacer functionality to cause displacement of edges 104 into a mutual non-touching relationship wherein said edges are in mutual propinquity and third tissue displacer functionality operative following application of energy to edges 104 to cause displacement of edges 104 of the tissue 102 into the mutual touching relationship along the seam for bonding thereof.

As seen in FIGS. 1A and 1B, the tissue displacer functionalities preferably include at least one tissue engagement displacer element, here shown schematically as first and second tissue engagement displacer elements respectively designated by reference numerals 108 and 110. Tissue engagement displacer elements, such as elements 108 and 110, may engage the tissue 102 in any suitable manner. One example is adhesive engagement directly between the tissue engagement displacer element and the underlying tissue. Alternatively, one or more wound dressings may be provided in adhesive engagement with the tissue and in adhesive or other engagement with one or more tissue engagement displacement elements. In accordance with a preferred embodiment of the present invention, at least one of, and preferably both, tissue engagement displacer elements 108 and 110 is transparent to radiant energy at least at a region thereof overlying edges 104.

Engagement between the tissue engagement displacer elements, the tissue and any intermediate element, such as a wound dressing, may be realized by the use of one or more adhesives, application of vacuum, use of staples or in any other suitable manner. It is noted that when a relatively deep cut is sought to be closed, surface engagement with the tissue is often not sufficient, thus requiring the use of staples, needles or other implements which provide the required depth of tissue engagement.

As seen in FIGS. 1A and 1B, at stages A and B, the first tissue displacer functionality is operative to initially close the wound. As seen particularly at stage B, the initial closure is not satisfactory and thus the operator, normally a surgeon, manipulates one or more of the tissue engagement displacer elements 108 and 110 so as to achieve a satisfactory closure, as seen at stage C. Once the satisfactory closure is achieved, the second tissue displacer functionality operates to axially displace the edges of the tissue 102 along a displacement axis 116 which is, in the illustrated case, perpendicular to the longitudinal axis 106 of the seam, to a mutual non-touching relationship wherein said edges 104 are in mutual propinquity, as seen at stage D.

It is appreciated that in one embodiment of the present invention, stage D and the following stages may be obviated, the edges 104 of the cut are retained in their mutually touching engagement by a suitable wound dressing and healing is allowed to progress from stage C without further manipulation.

Turning now to stage E, there is seen a tissue bonding enhancement material dispenser 120 preferably operative, following operation of the second tissue displacer functionality, to apply a tissue bonding enhancement material 122, preferably human serum albumin or, alternatively, any other suitable tissue bonding enhancement material, to edges 104 when edges 104 are in mutual non-touching relationship but in mutual propinquity as seen at stage E. It is appreciated that in one embodiment of the present invention stage E may be obviated or may be repeated further in the sequence of FIGS. 1A and 1B.

Turning now to stage F, it is seen that normally following application of the tissue bonding enhancement material 122, tissue-bonding functionality is operative to apply energy to the edges 104 of tissue 102. In a preferred embodiment of the invention, tissue-bonding functionality comprises a 10 W CO2 laser 130 coupled via an optical fiber 132 to a linear scanner or lens 134. It is appreciated that, alternatively, any other suitable laser or other energy source may be employed. Examples of suitable alternative energy sources include a YAG laser, microwave transmitter, IR transmitter, UV transmitter, ultra sound or others for the heating and the excitation of the soldering agent and the heating of the tissue.

Normally, during or following application of energy to the edges 104 of tissue 102, third tissue displacer functionality is operative to cause displacement of the edges 104 of tissue 102 into mutual touching relationship along the seam for bonding thereof. This is seen at stage G. As will be described in greater detail hereinbelow, when a deep cut is involved, application of energy and closure preferably takes place layer by layer from the inside out. Application of tissue bonding enhancement material 122, as in stage E, may be repeated one or more times during or between applications of energy to edges 104.

Stage H shows the bonded cut 140. It is appreciated that the edges (not shown) of the cut are retained in their mutually touching engagement shown at stage H by a suitable wound dressing and healing is allowed to progress from stage H without further manipulation. Tissue engagement displacer elements, such as elements 108 and 110, may be disengaged from the tissue at this stage.

It is appreciated that the functionality of FIGS. 1A and 1B may be operated remotely by using any suitable data link.

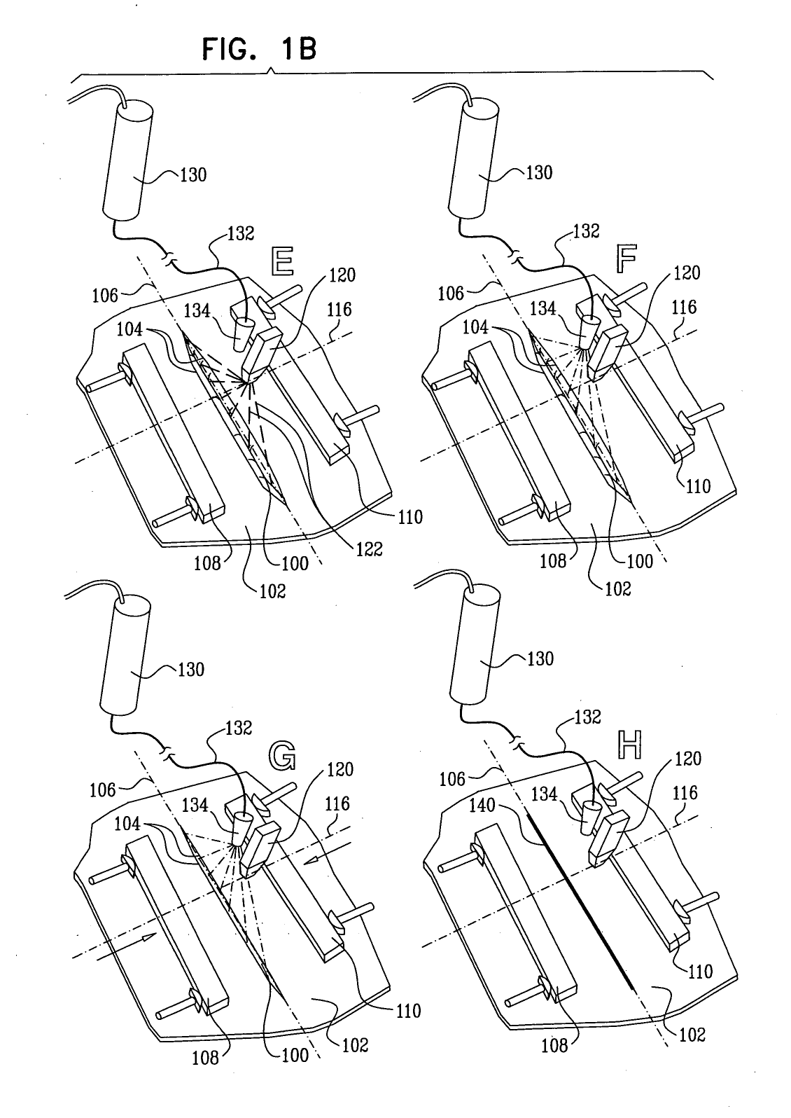

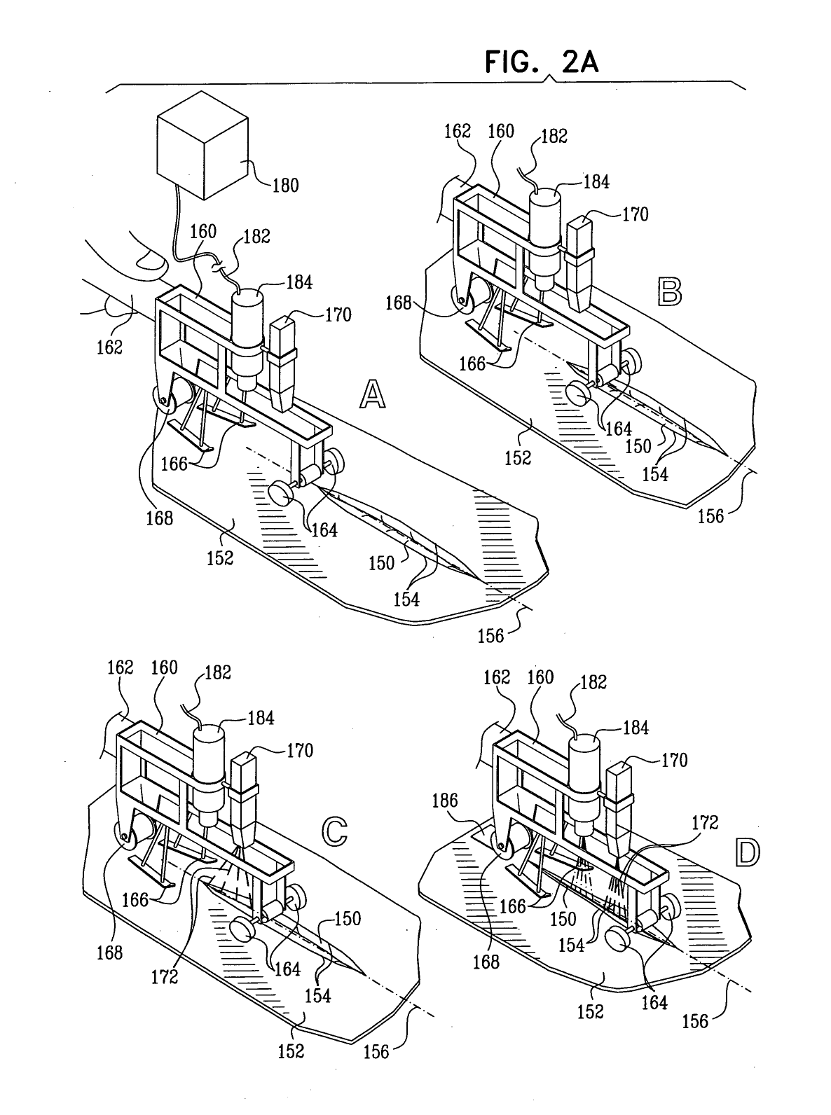

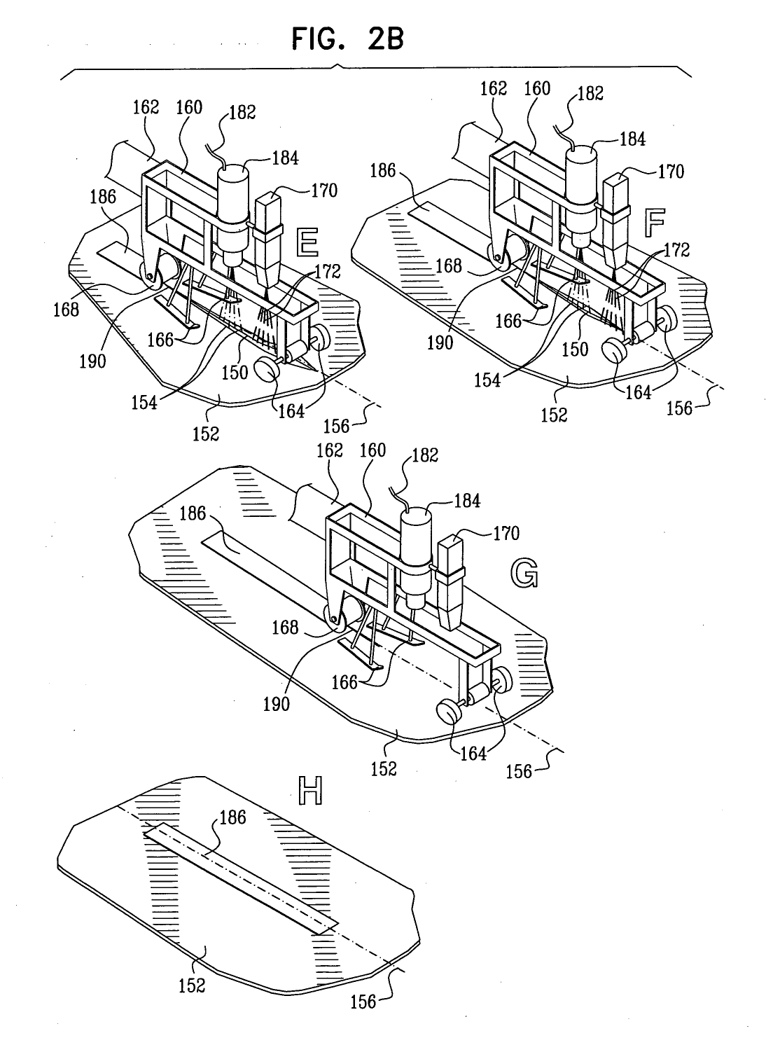

Reference is now made to FIGS. 2A and 2B, which are, together, a simplified illustration of the operation of a system and method for bonding living tissue in accordance with another preferred embodiment of the present invention. The embodiment of FIGS. 2A and 2B is distinguished from that of FIGS. 1A and 1B in that in the embodiment of FIGS. 2A and 2B, a cut 150 in tissue 152 is bonded serially therealong, from end to end, while in the embodiment of FIGS. 1A and 1B, it is bonded in parallel along at least a portion of its length. Additionally, whereas the embodiment of FIGS. 1A and 1B employs tissue engagement displacer elements which are attached to the tissue adjacent edges of the cut, in the embodiment of FIGS. 2A and 2B, the tissue engagement displacer elements move along the length of the cut 150 parallel to edges 154 along a seam axis 156.

The embodiment of FIGS. 2A and 2B preferably includes a chassis 160 which is attached to a handle 162 suitable for being held in the hand of an operator, such as a surgeon. Mounted on chassis are preferably a pair of forward rollers 164, which preferably have two functions: driving the chassis 160 at a controlled velocity over the cut 150 along the surface of tissue 152 and sequentially separating edges 154 of cut 150 as the chassis moves along the surface of tissue 152.

Mounted on chassis 160 rearward of forward rollers 164 are a pair of slider elements 166 which preferably are operative to provide displacement of edges 154 of tissue 152 into mutual touching relationship along a seam extending along seam axis 156 following application of energy to edges 154 for bonding thereof. It is appreciated that forward rollers 164 and slider elements 166 may typically be interchangeable, thus the functionality of forward rollers 164 may be provided by slider elements and the functionality of slider elements 166 may be provided by rollers.

Mounted on chassis 160 rearward of slider elements 166 is a wound dressing tape dispensing roller 168, which is operative to dispense a wound dressing tape and to cause it to engage tissue 152 over the bonded edges 154 and to retain them in mutual touching relationship.

Mounted on chassis 160 and maintained at a fixed, predetermined distance from the surface of tissue 152 is a tissue bonding enhancement material dispenser 170 preferably operative, downstream of rollers 164, to apply a tissue bonding enhancement material 172, preferably human serum albumin or, alternatively, any other suitable tissue bonding enhancement material, to edges 154 when edges 154 are in mutual non-touching relationship but in mutual propinquity due to the action of rollers 164.

Also mounted on chassis 160 and maintained at a fixed, predetermined distance from the surface of tissue 152 is tissue bonding functionality operative to apply energy to edges 154 of tissue 152. In a preferred embodiment of the invention, tissue-bonding functionality comprises a 10 W CO2 laser 180 coupled via an optical fiber 182 to a linear scanner or lens 184. It is appreciated that, alternatively, any other suitable laser or other energy source may be employed. Examples of suitable alternative energy sources include a YAG laser, microwave transmitter, IR transmitter, UV transmitter, ultra sound or others for the heating and the excitation of the soldering agent and the heating of the tissue.

Turning now to the various stages of operation of the functionality of FIGS. 2A and 2B, stage A shows handle 162 is in the hand of the surgeon, being positioned such that rollers 164 and slider elements 166 engage a surface of tissue 152 and chassis 160 is aligned with seam axis 156 downstream of cut 150.

Stage B shows initial operative engagement of rollers 164 with cut 150 for separating the edges 154 of cut 150 such that edges 154 lie in mutual non-touching relationship but in mutual propinquity.

Stage C shows further linear displacement of chassis 160 along cut 150, with rollers 164 continuing to separate the edges 154 of cut 150 such that edges 154 lie in mutual non-touching relationship but in mutual propinquity and tissue bonding enhancement material dispenser 170 being operative, downstream of rollers 164, to apply a tissue bonding enhancement material 172 to edges 154.

Stage D shows still further linear displacement of chassis 160 along cut 150, with rollers 164 continuing to separate the edges 154 of cut 150 such that edges 154 lie in mutual non-touching relationship but in mutual propinquity, tissue bonding enhancement material dispenser 170 being operative downstream of rollers 164, to apply a tissue bonding enhancement material 172 to edges 154 and laser 180 of tissue bonding functionality being operative, downstream of dispenser 170, to apply energy to edges 154 of tissue 152. Wound dressing tape dispensing roller 168 is seen to be operative to dispense a wound dressing tape 186 and to cause it to engage tissue 152 downstream of the bonded edges 154.

Stage E shows yet further linear displacement of chassis 160 along cut 150, with rollers 164 continuing to separate the edges 154 of cut 150 such that edges 154 lie in mutual non-touching relationship but in mutual propinquity, tissue bonding enhancement material dispenser 170 being operative downstream of rollers 164, to apply a tissue bonding enhancement material 172 to edges 154 and laser 180 of tissue bonding functionality being operative, downstream of dispenser 170, to apply energy to edges 154 of tissue 152. Slider elements 166 are seen to be operative to provide displacement of edges 154 of tissue 152 into mutual touching relationship along a seam 190, extending along seam axis 156, following application of energy to edges 154 for bonding thereof. Wound dressing tape dispensing roller 168 is seen to be operative to dispense wound dressing tape 186 and to cause it to engage tissue 152 over seam 190.

Stage F shows additional linear displacement of chassis 160 along cut 150, with rollers 164 being located upstream of cut 150, tissue bonding enhancement material dispenser 170 being operative, downstream of rollers 164, to apply a tissue bonding enhancement material 172 to edges 154 and laser 180 of tissue bonding functionality being operative, downstream of dispenser 170, to apply energy to edges 154 of tissue 152. Slider elements 166 are seen to be operative to provide displacement of edges 154 of tissue 152 into mutual touching relationship along seam 190, extending along seam axis 156, following application of energy to edges 154 for bonding thereof. Wound dressing tape dispensing roller 168 is seen to be operative to dispense wound dressing tape 186 and to cause it to engage tissue 152 over seam 190.

Stage G shows still further linear displacement of chassis 160 along cut 150, with rollers 164, tissue bonding enhancement material dispenser 170 and laser 180 of tissue bonding functionality being located upstream of cut 150 (not shown). Slider elements 166 are seen to be operative to provide displacement of edges 154 of tissue 152 into mutual touching relationship along the seam 190, extending along seam axis 156, following application of energy to edges 154 for bonding thereof. Wound dressing tape dispensing roller 168 is seen to be operative to dispense wound dressing tape 186 and to cause it to engage tissue 152 over seam 190.

Stage H shows cut 150 following bonding thereof and covered by wound dressing tape 186, which retains edges 154 (not shown) in mutual touching engagement along seam 190.

It is appreciated that the various elements of the embodiment of FIGS. 2A and 2B need not necessarily all be provided. For example, rollers 164 and their functionality may be obviated in certain cases. As another example, wound-dressing tape dispensing roller 168 and its functionality need not be part of the present invention.

It is appreciated that although the illustrations of FIGS. 1A-2B illustrate straight cuts, the present invention is not limited to use with straight cuts and can be employed with cuts of any suitable configuration which may contain multiple curves in three dimensions.

Reference is now made to FIG. 3, which is a simplified pictorial view illustration of the system of FIG. 1A constructed and operative in accordance with one embodiment of the present invention in an operative orientation on a patient. As seen in FIG. 3, there is provided a system for bonding living tissue in operative association with a patient. The system preferably includes a patient-mounted portion 200 and a non patient-mounted portion 202, typically mounted on a cart 204.

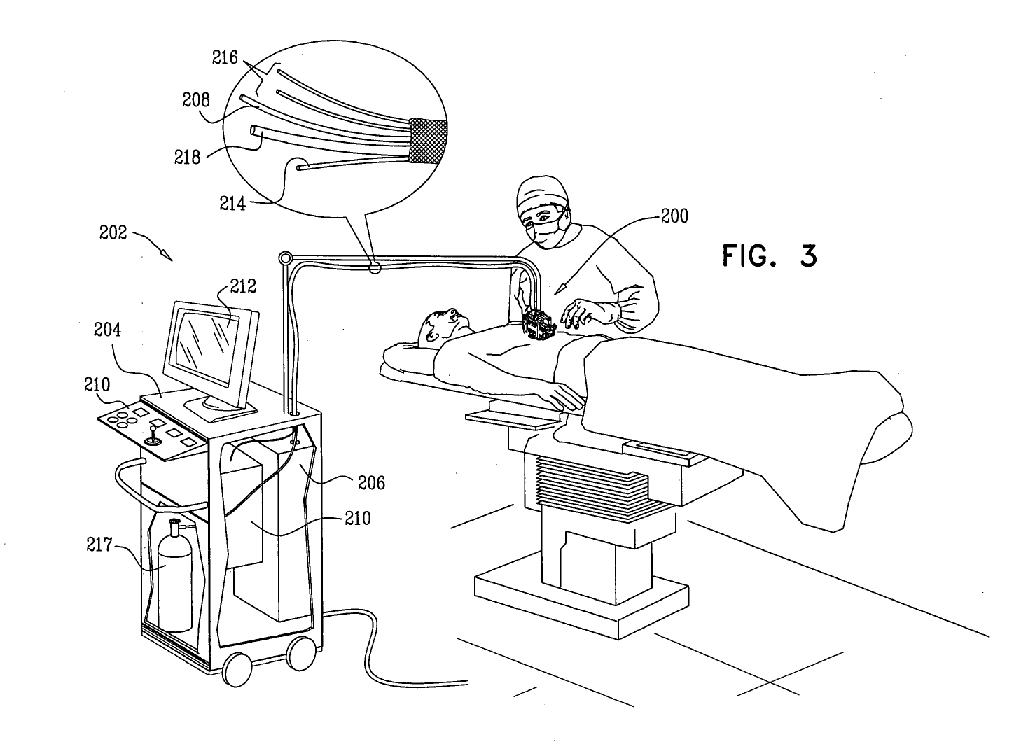

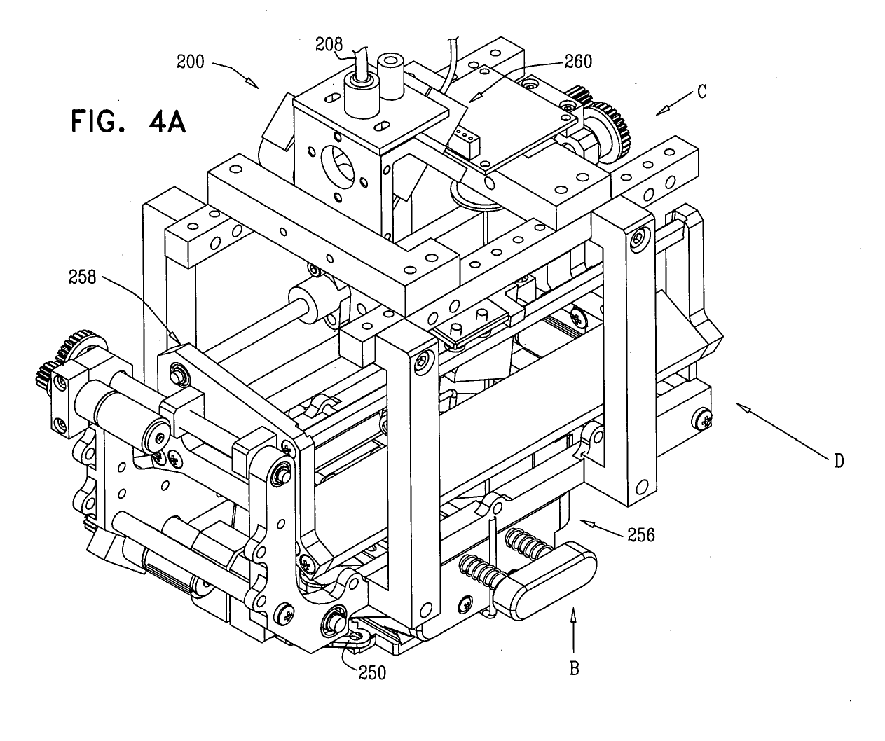

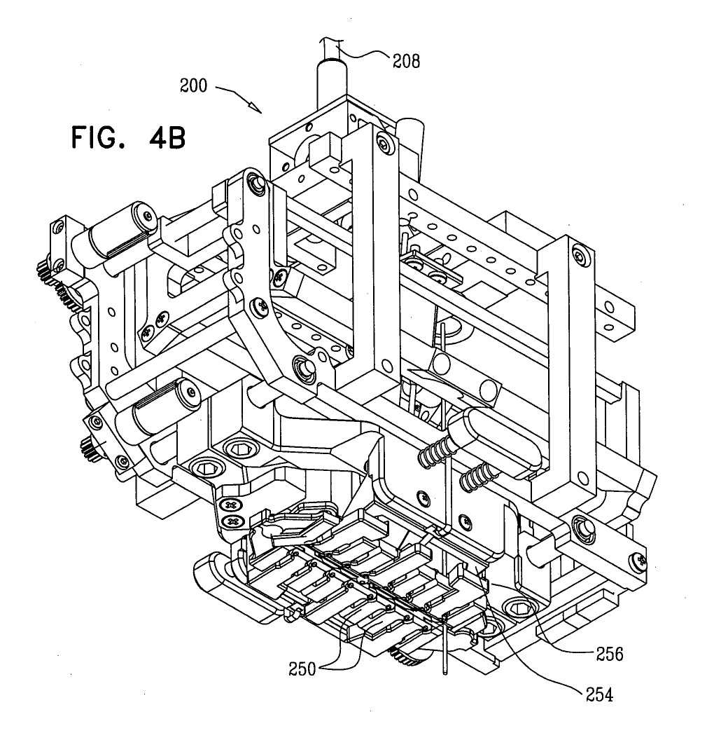

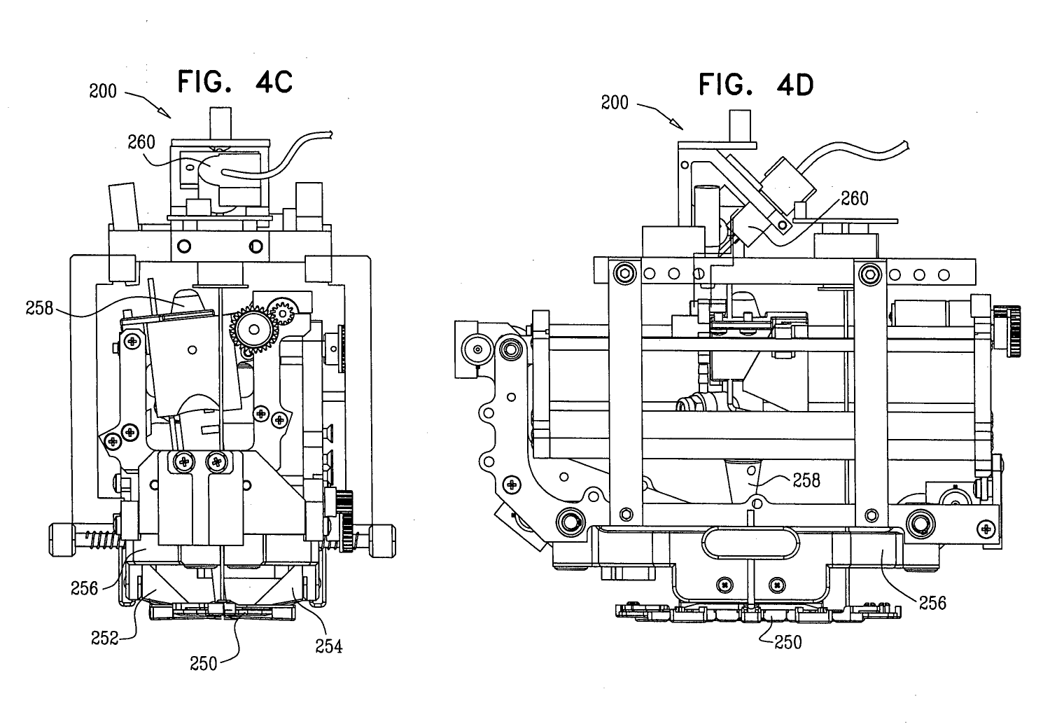

The patient-mounted portion 200 preferably is supported by a patient's body and is located over a cut to be bonded. The patient-mounted portion 200 is described hereinbelow in detail with reference to FIGS. 4A-26C and preferably includes first tissue displacer functionality operative initially to cause displacement of edges of tissue into a mutual touching relationship along a seam prior to bonding thereof, second tissue displacer functionality operative following operation of the first tissue displacer functionality to cause displacement of the edges into a mutual non-touching relationship wherein the edges are in mutual propinquity, tissue bonding functionality operative following operation of the first and second displacer functionalities to apply energy to the edges of the tissue and third tissue displacer functionality operative to cause displacement of said edges of the tissue into the mutual touching relationship along the seam for bonding thereof and a tissue bonding enhancement material dispenser operative following operation of the second tissue displacer functionality and at least partially prior to the tissue bonding functionality to apply a tissue bonding enhancement material to the edges when the edges are in the mutual non-touching relationship wherein the edges are in mutual propinquity.

The non patient-mounted portion 202 preferably includes a laser 206, preferably a 10 W CO₂ laser having a coherent light output, coupled to a flexible optical fiber 208, which is in turn coupled to the patient-mounted portion 200. A computerized control unit 210, having associated therewith an operator interface screen 212, controls the operation of laser 206 and provides control outputs to the patient-mounted portion 200, preferably via a multi-conductor data cable 214. The computerized control unit preferably receives a video feed from a video camera forming part of the patient-mounted portion 200 via a coaxial data cable 216. Computerized control unit preferably also receives, via data cable 214, a temperature input from patient-mounted portion 200 and a bonded tissue tensile force input from a tissue tension force sensor forming part of the patient-mounted portion 200, indicating the amount of tensile force applied to the bonded edges of the tissue when displaced into the mutually touching relationship. Optionally, a source of compressed gas 217 may provide compressed gas to the patient-mounted portion 200 via a flexible compressed gas conduit 218.

Reference is now made to FIGS. 4A, 4B, 4C and 4D, which are simplified pictorial and plan view illustrations of a patient-mounted portion of the system of FIG. 3, FIG. 4B being an underside plan view taken along arrow B in FIG. 4A, FIGS. 4C and 4D being respective end and side views taken along respective arrows C and D in FIG. 4A, and to FIG. 5, which is a simplified partially exploded view illustration of the patient-mounted portion of FIGS. 4A-4D.

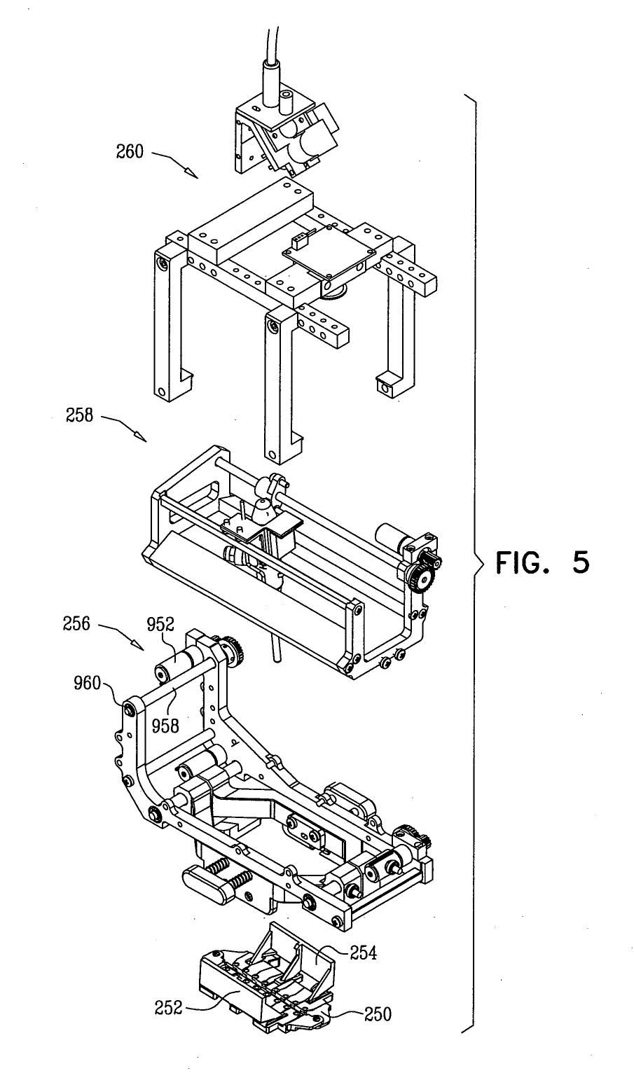

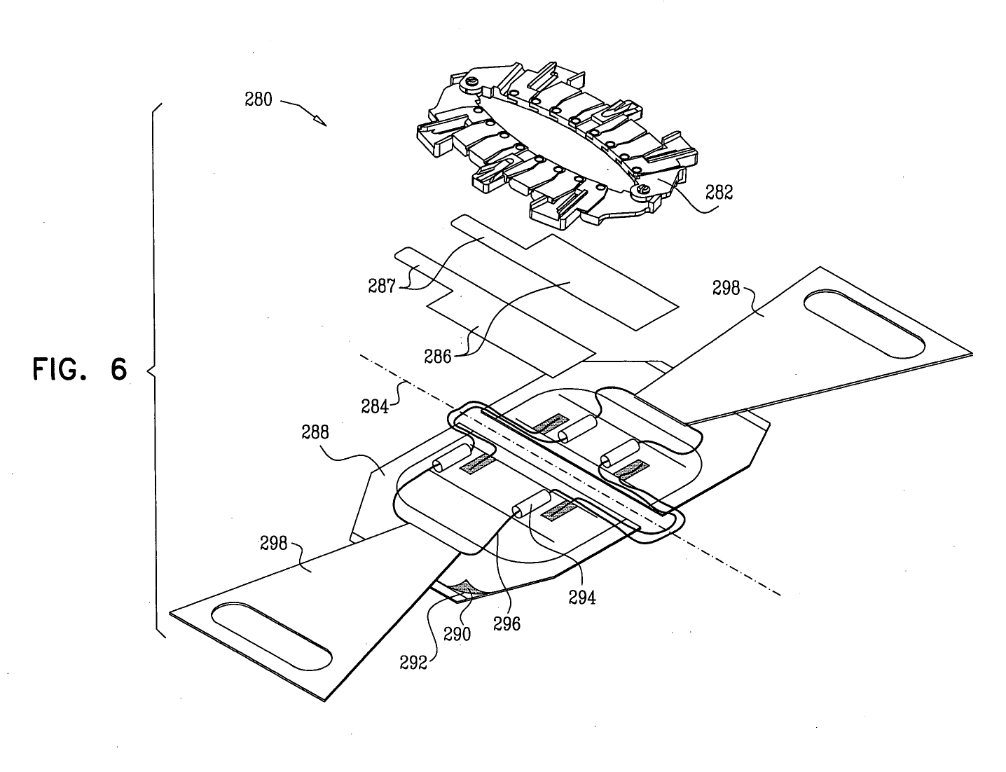

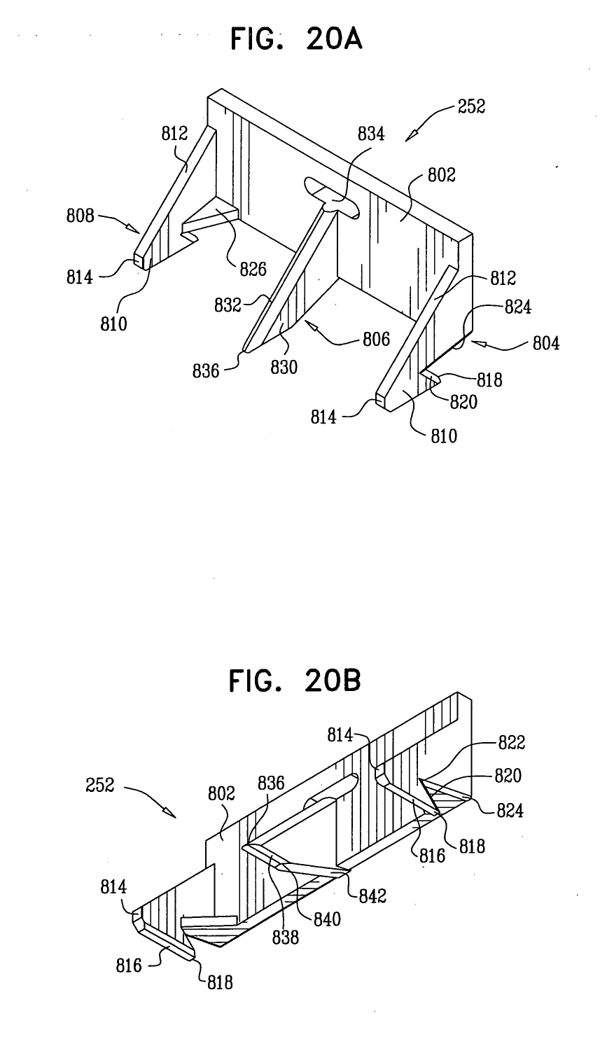

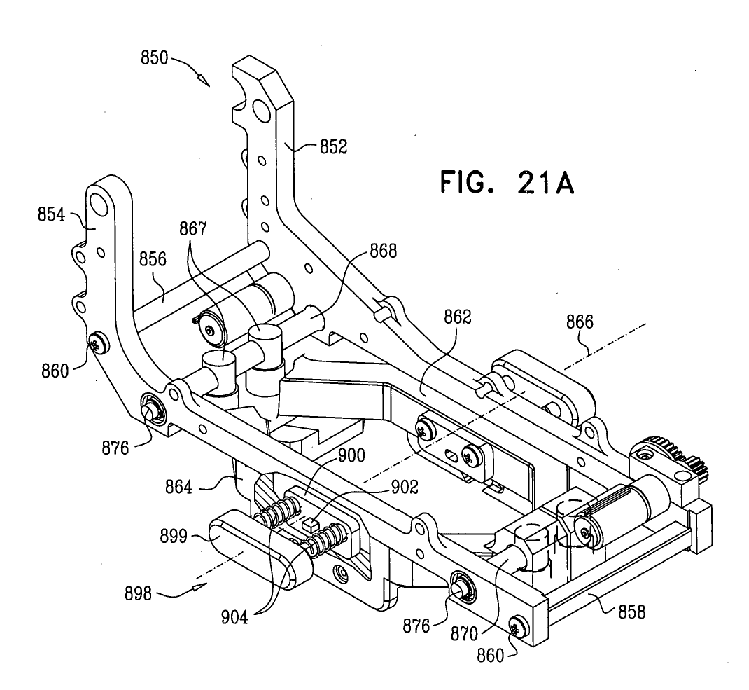

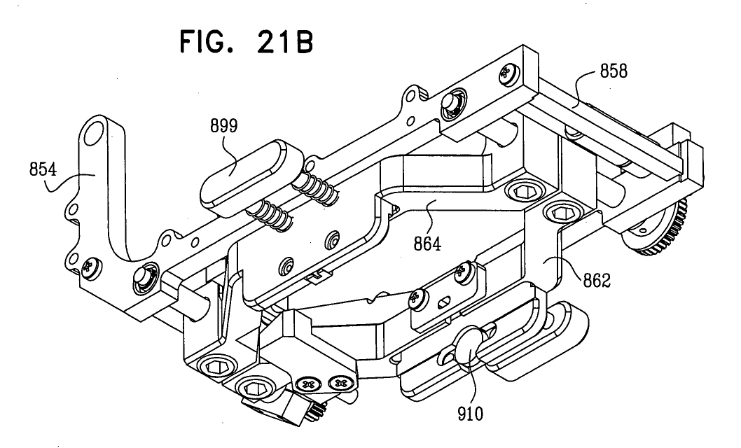

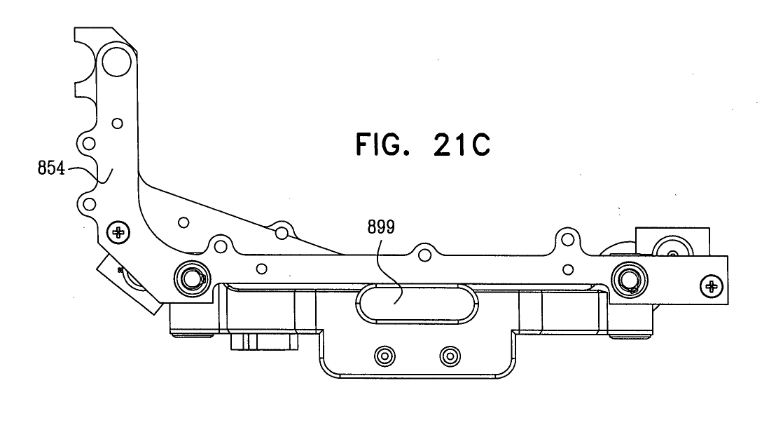

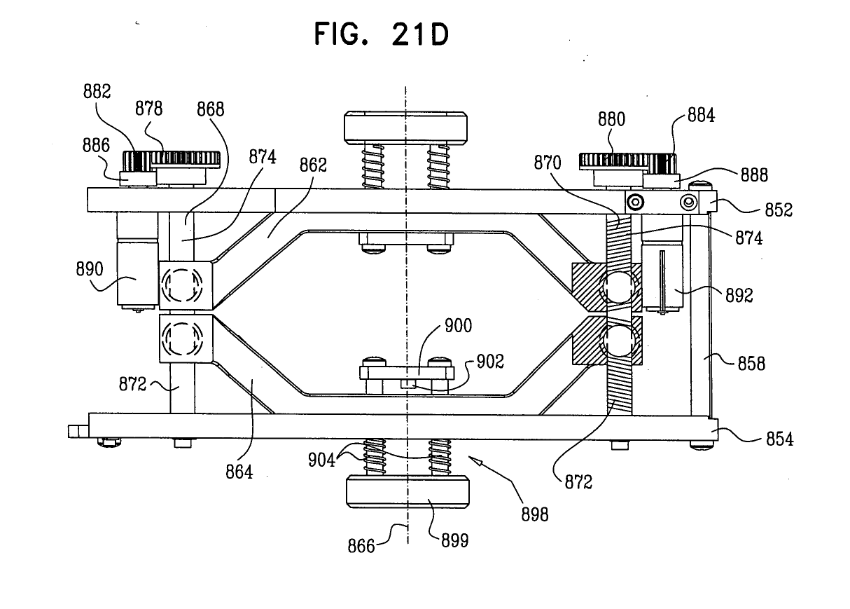

As seen in FIGS. 4A-4D and 5, the patient-mounted portion 200 includes a clamp portion 250 of a clamp assembly, which is described hereinbelow in detail with reference to FIG. 6. The clamp portion 250 is engaged by a pair of clamp engagement elements 252 and 254, described hereinbelow with reference to FIGS. 20A and 20B, which are, in turn, manipulated by a clamp manipulation subassembly 256, which is described herein below in detail with reference to FIGS. 21A-21D.

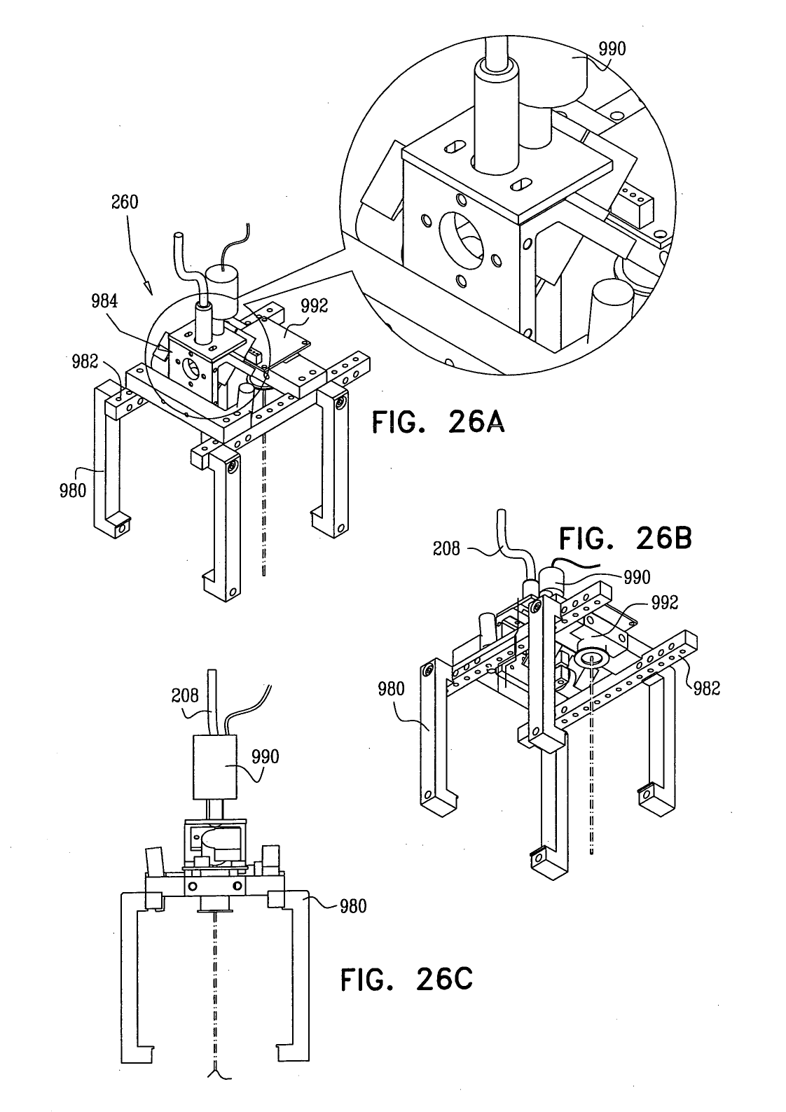

Mounted on clamp manipulation subassembly 256 there is preferably provided a tissue bonding enhancement material dispenser subassembly 258, which is described herein below in detail with reference to FIGS. 24A and 24B. Also preferably mounted on the clamp manipulation subassembly 256 is an energy directing subassembly 260, to which is coupled flexible optical fiber 208. Energy directing subassembly 260 is described herein below in detail with reference to FIGS. 26A-26C.

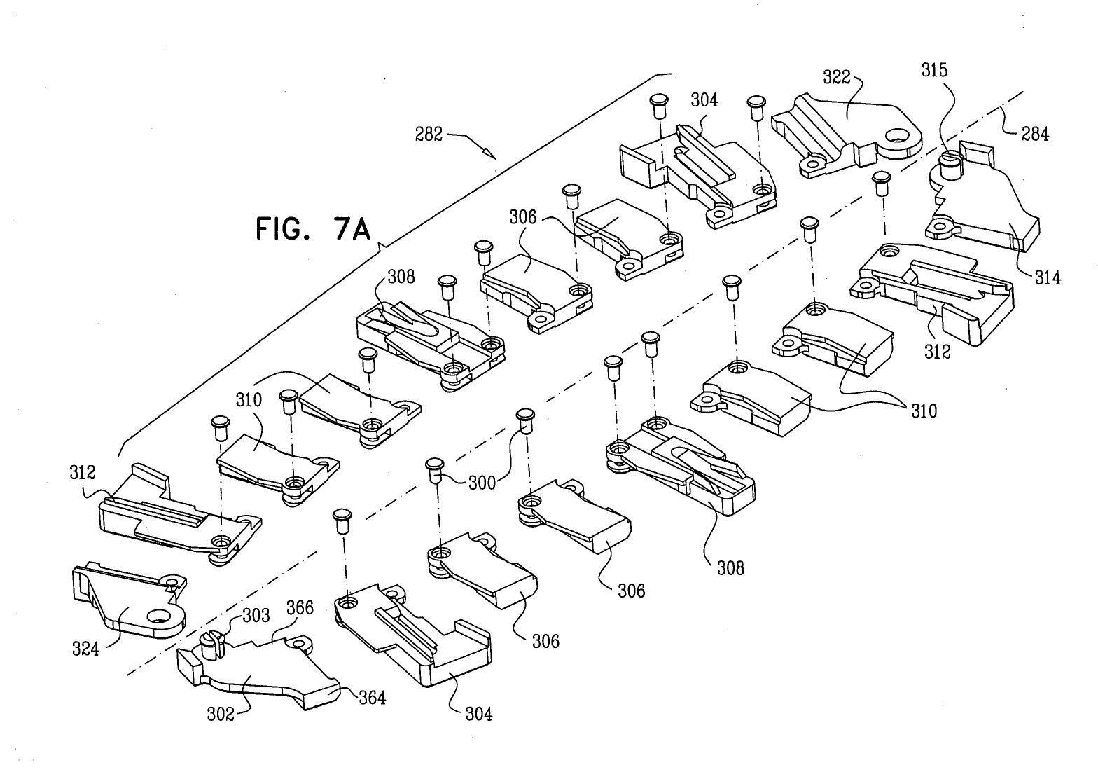

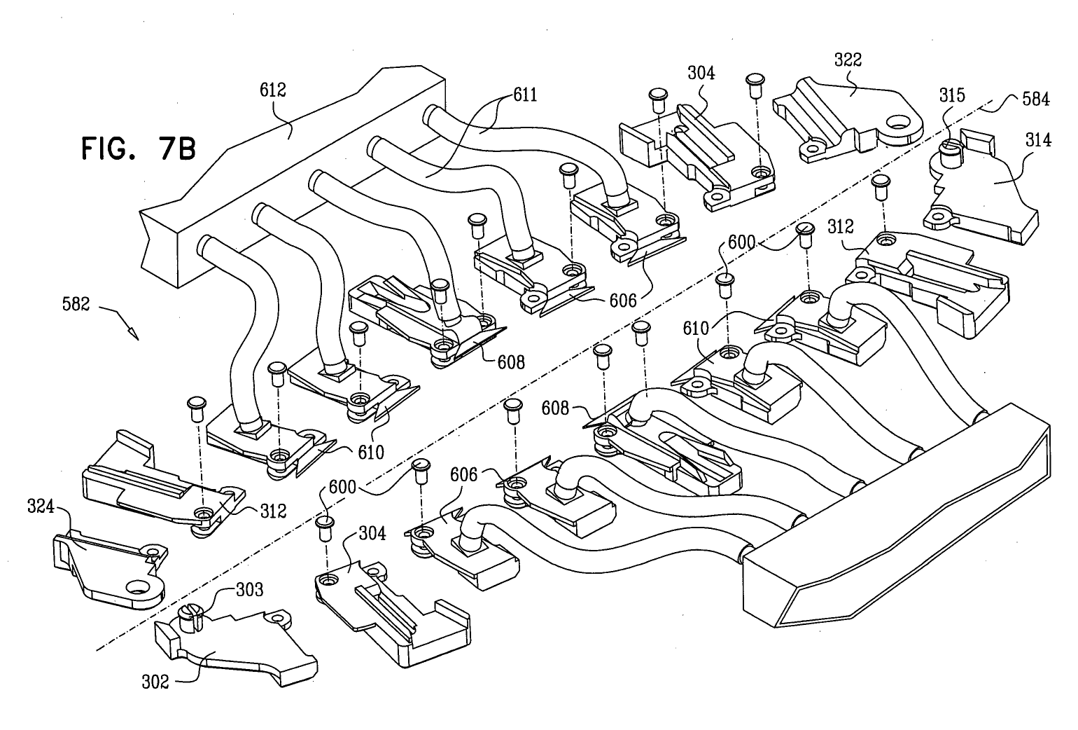

Reference is now made to FIG. 6, which is a simplified partially exploded view illustration of a clamp assembly forming part of the system of FIG. 3. As noted above, the clamp assembly, here designated by reference numeral 280, includes a clamp portion 282 having a longitudinal axis 284, which is described herein below in detail with reference to FIGS. 7A-19F. Adhesively mounted to each side of the underside of clamp portion 282 is a flexible substrate 286 having tabs 287. Removably adhesively bonded to each flexible substrate 286 is a side bandage portion 288 having an adhesive surface 290 for attachment to a patient's body and a release layer 292. Fixed to each bandage portion 288 is a plurality of conduits 294 extending generally perpendicularly to longitudinal axis 284. A plurality of elongate elements 296 each extends through a corresponding one of the plurality of conduits 294. One end of each of the elongate elements 296 extending through conduits 294 on each side of clamp portion 282 is connected to a common pull-tab 298. The opposite end of each of the elongate elements 296 extending through conduits 294 on each side of clamp portion 282 is fixed to a side bandage portion 288 on an opposite side of the clamp portion 282.

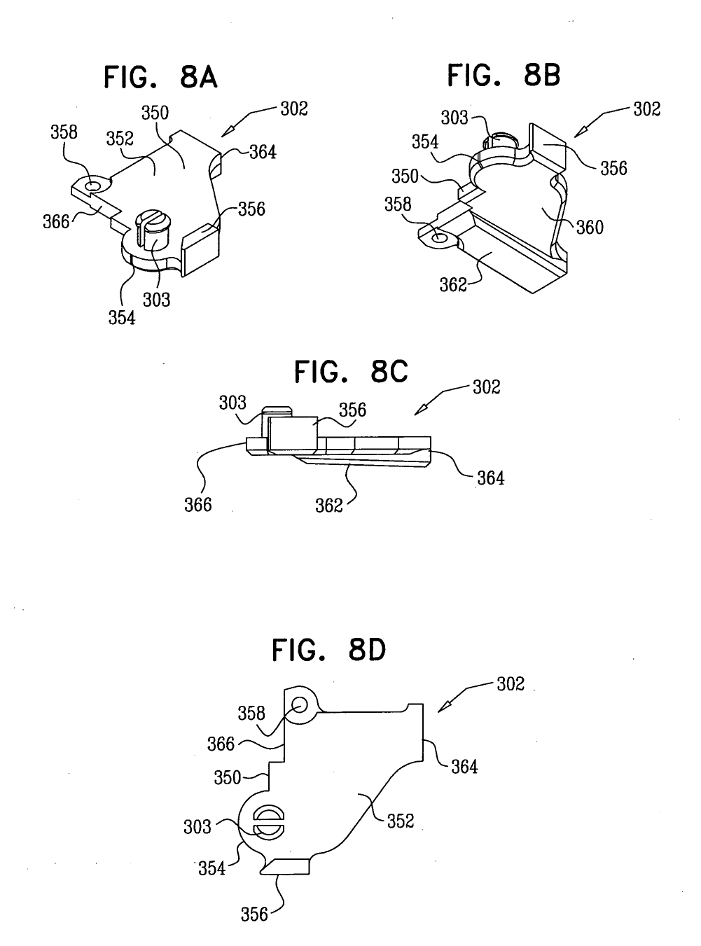

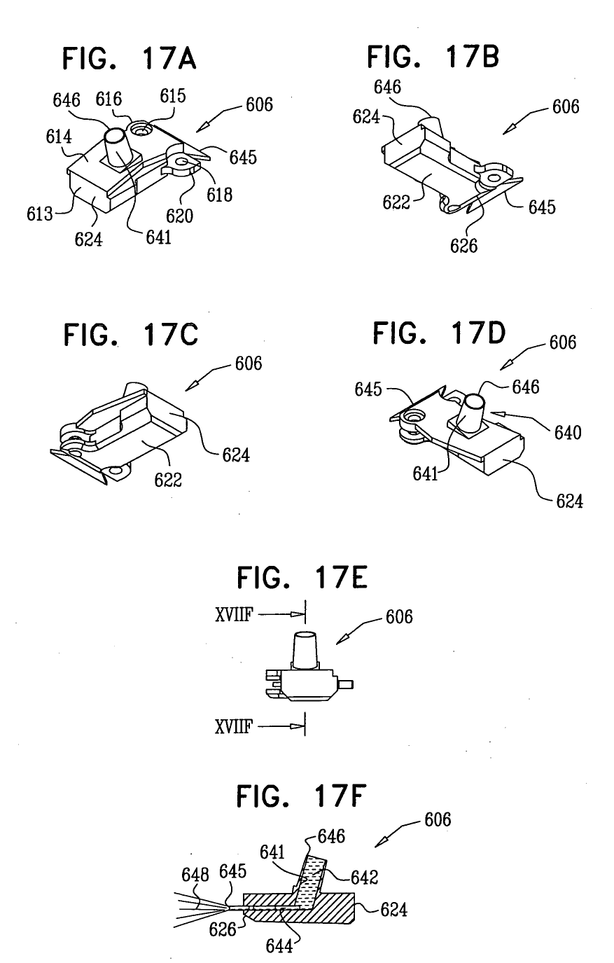

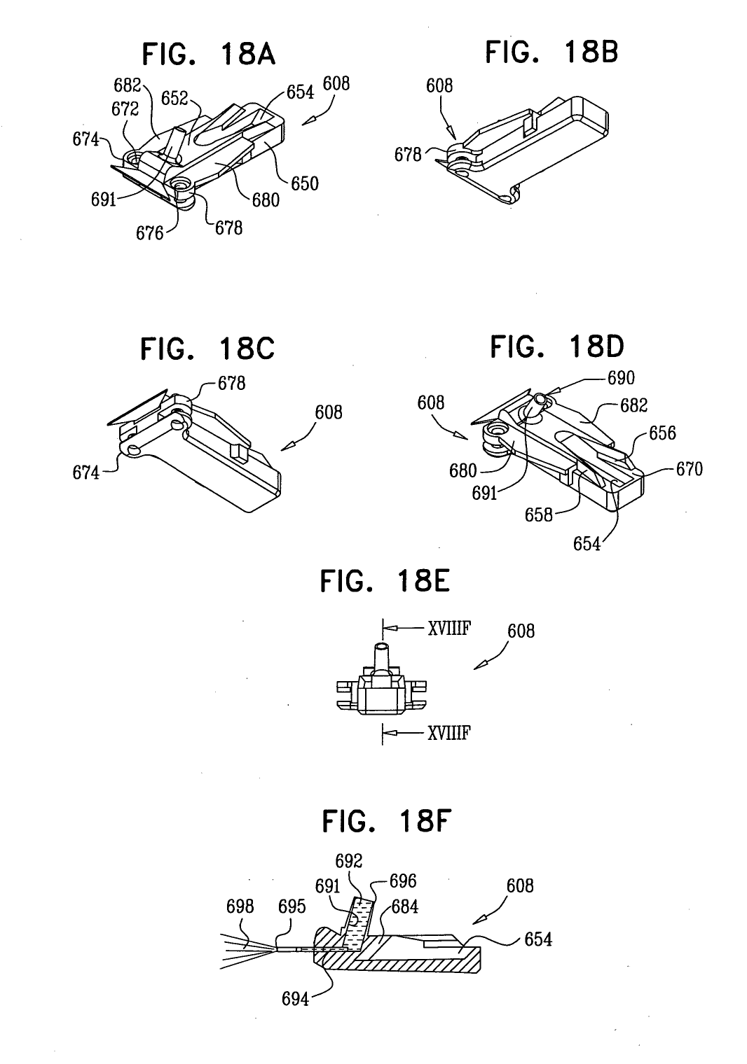

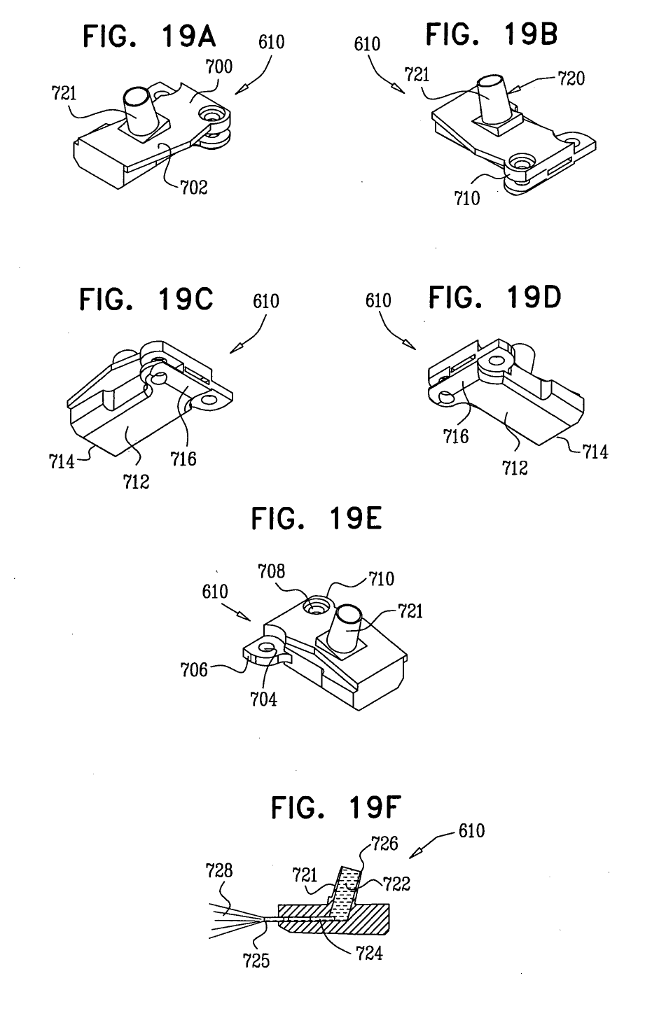

Reference is now made to FIGS. 7A and 7B, which are each a simplified exploded view illustration of clamp portion 282 of the clamp assembly of FIG. 6 in accordance with two alternative embodiments of the present invention, to FIGS. 8A-16E, which illustrate individual parts of the clamp portion of FIG. 7A, and to FIGS. 17A-19F, which illustrate individual parts of the clamp portion of FIG. 7B which are not identical to those in the clamp portion of FIG. 7A.

Turning to FIG. 7A, it is seen that clamp portion 282 is comprised of a multiplicity of parts, most of which are pivotably joined together by pins 300. Clamp portion 282 is generally symmetric about longitudinal axis 284, but, as will be appreciated, is not precisely symmetric about axis 284. As seen in FIG. 7A, the clamp portion includes, at a first side thereof, with respect to axis 284, a first part 302 having an integrally formed bifurcated pin 303, a second part 304, a pair of adjacent third parts 306, a fourth part 308, a pair of adjacent fifth parts 310, a sixth part 312 and a seventh part 314, having an integrally formed pin 315. The clamp portion 282 also includes, at a second side thereof, with respect to axis 284, an eighth part 322, a second part 304, a pair of adjacent third parts 306, a fourth part 308, a pair of adjacent fifth parts 310, a sixth part 312 and a ninth part 324.

Reference is now made specifically to FIGS. 8A, 8B, 8C and 8D which are each a simplified illustration of first part 302 of the clamp portion of FIG. 7A. As seen in FIGS. 8A, 8B, 8C and 8D, first part 302 includes a generally planar element 350 having bifurcated pin 303 extending generally perpendicularly from a top surface 352 thereof adjacent a rounded corner 354. Adjacent and spaced from bifurcated pin 303, a motion limiter 356 extends perpendicular to top surface 352. Opposite motion limiter 356, a pin socket 358 extends outwardly from generally planar element 350 and is somewhat recessed with respect to top surface 352. On a bottom surface 360 of generally planar element 350 there is defined an inclined portion 362, which has decreasing thickness from an outer facing edge 364 to an inner facing edge 366.

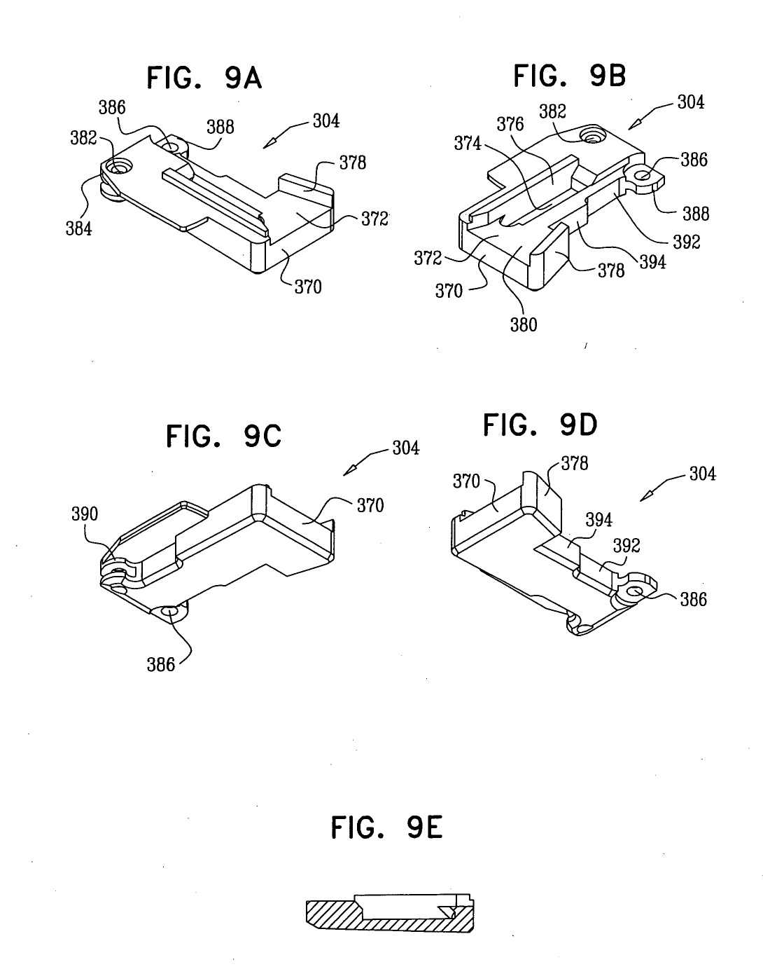

Reference is now made specifically FIGS. 9A, 9B, 9C, 9D and 9E, which are each a simplified illustration of second part 304, two of which are arranged on opposite sides of the clamp portion of FIG. 7A with respect to axis 284. As seen in FIGS. 9A, 9B, 9C, 9D and 9E, second part 304 includes a generally planar element 370 having a top surface 372 defining an elongate socket 374 extending generally perpendicularly to longitudinal axis 284. First and second elongate guiding walls 376 and 378 extend perpendicularly to top surface 372 along opposite sides of elongate socket 374 to define a converging top surface tooth directing region 380 adjacent socket 374. A first pin socket 382 is defined at a first corner 384 of top surface 372 and a second pin socket 386 is defined at an adjacent corner 388 of top surface 372 and is somewhat recessed with respect to top surface 372. An overlappable surface portion 390 of top surface 372 extends below corner 384 for providing radiant energy shielding when the clamp portion is partially open. One or both edges 392 and 394 of generally planar element 370 may define a relative motion limiter with respect to an adjacent third part 306.

Reference is now made to FIGS. 10A, 10B, 10C and 10D, which are each a simplified illustration of third part 306 of the clamp portion of FIG. 7A, four of which, arranged in side by side pairs on opposite sides of axis 284, form part of the clamp portion of FIG. 7A. As seen in FIGS. 10A, 10B, 10C and 10D, third parts 306 each include a generally planar element 400 having a top surface 402. A first pin socket 404 is provided adjacent a corner 406 of planar element 400 and a second pin socket 408 is provided adjacent an opposite corner 410. A bottom surface 412 of generally planar element 400 is inclined such that planar element 400 has decreasing thickness from an outer facing edge 414 to an inner facing edge 416 thereof.

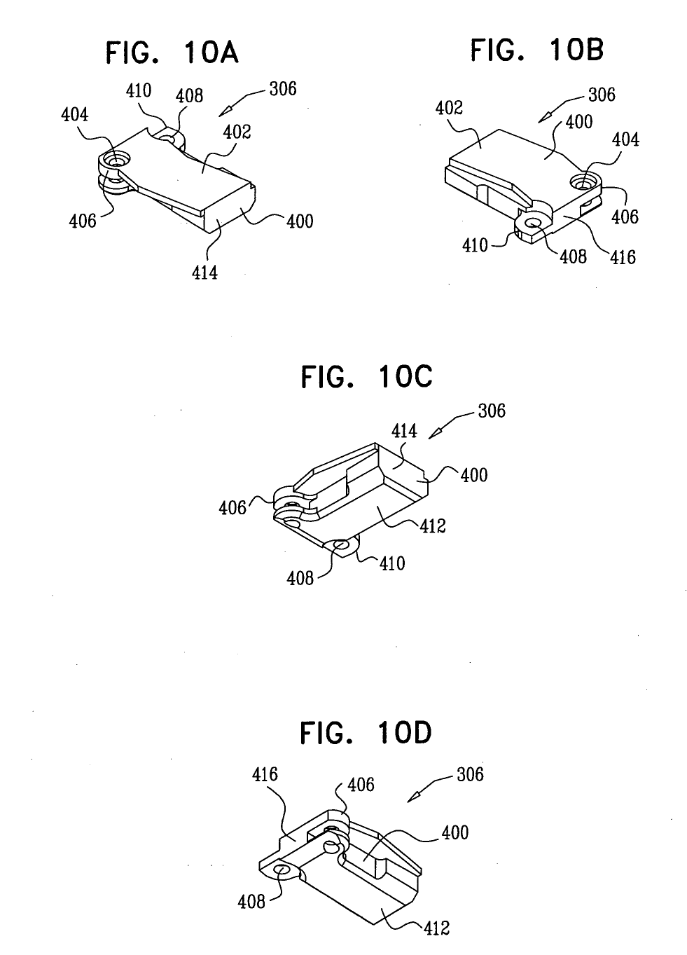

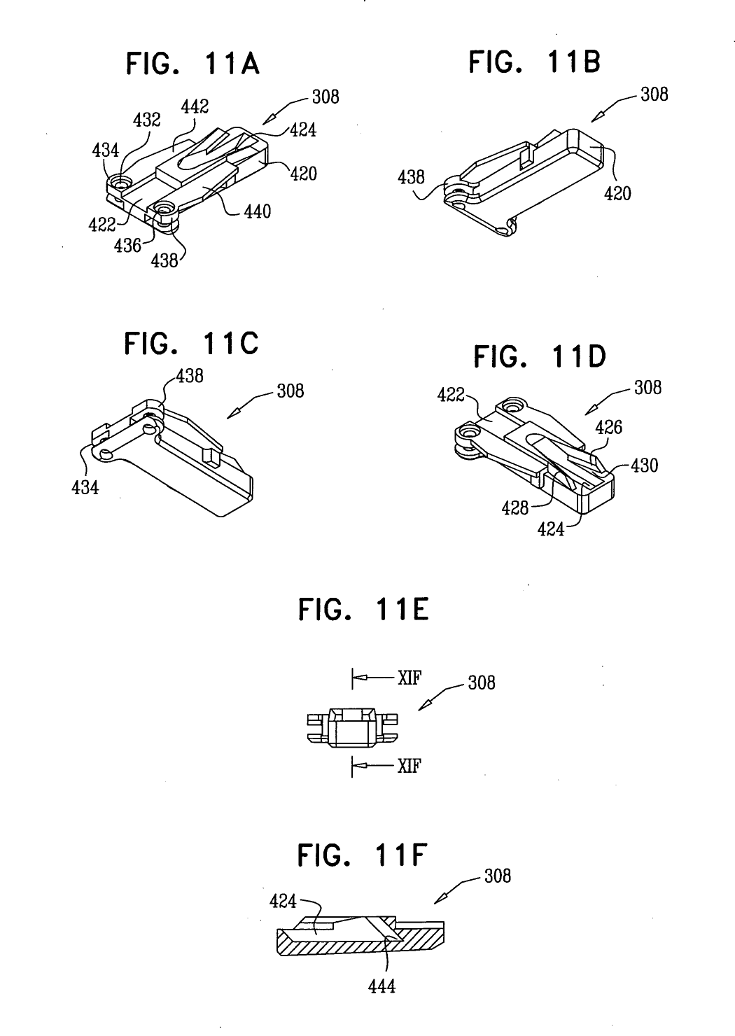

Reference is now made to FIGS. 11A, 11B, 11C, 11D, 11E and 11F, which are each a simplified illustration of fourth part 308, two of which are arranged on opposite sides of the clamp portion of FIG. 7A with respect to axis 284. As seen in FIGS. 11A, 11B, 11C, 11D, 11E and 11F, part 308 includes a generally planar element 420 having a top surface 422 defining an elongate socket 424 extending generally perpendicularly to longitudinal axis 284. First and second elongate guiding walls 426 and 428 extend perpendicularly from top surface 422 along opposite sides of elongate socket 424 to define a converging top surface tooth directing region 430 adjacent socket 424. A first pin socket 432 is defined at a first corner 434 of top surface 422 and a second pin socket 436 is defined at an adjacent corner 438 of top surface 422. Overlappable surface portions 440 and 442 of top surface 422 extend below respective corners 434 and 438 for providing radiant energy shielding when the clamp portion is partially open. It is a particular feature of the structure of part 308 that an inner facing end surface 444 of elongate socket 424 is undercut.

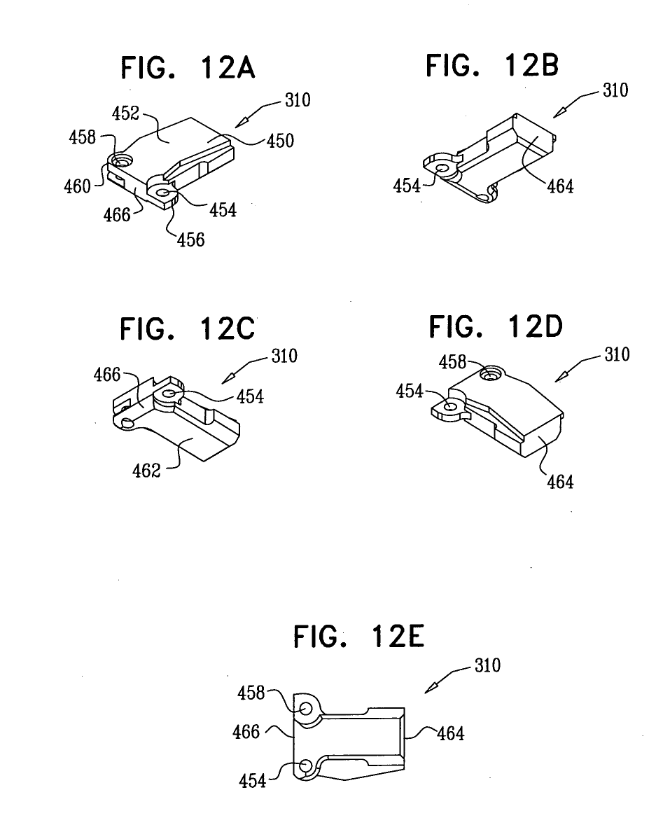

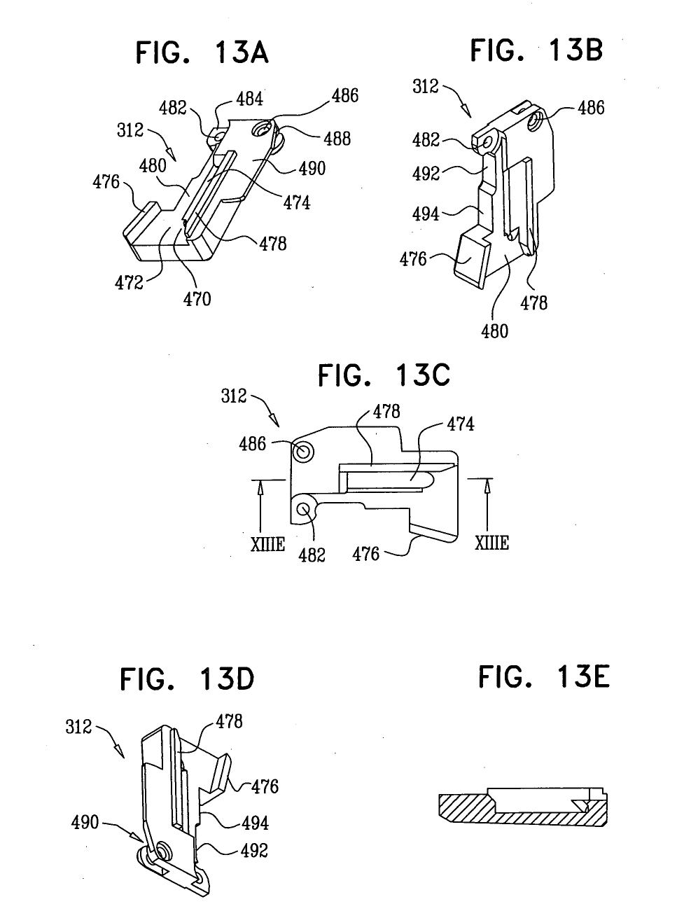

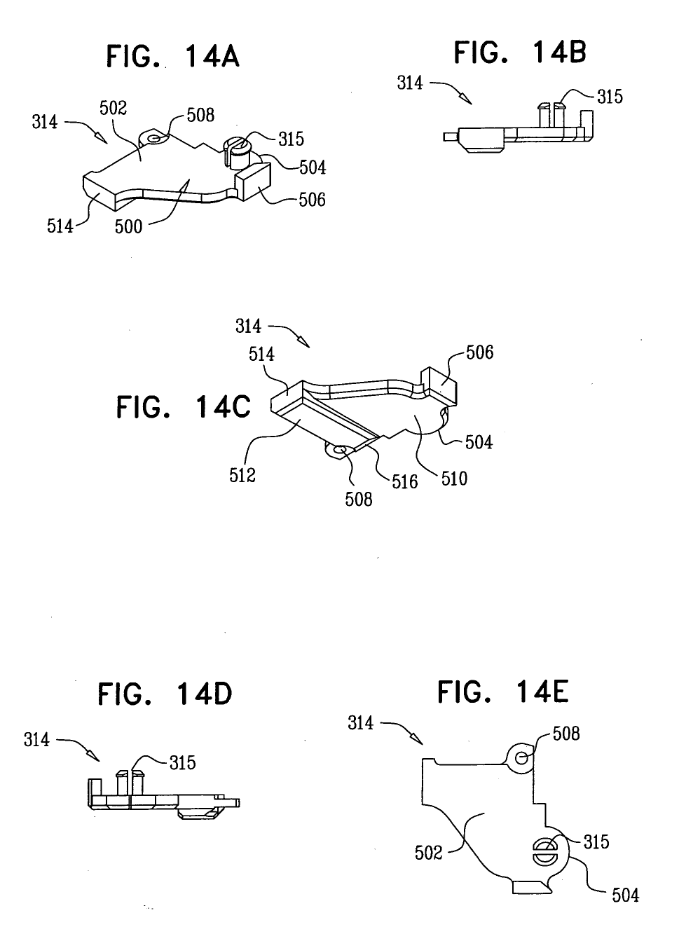

Reference is now made to FIGS. 12A, 12B, 12C, 12D and 12E, which are each a simplified illustration of fifth part 310 of the clamp portion of FIG. 7A, four of which, arranged in side by side pairs on opposite sides of axis 284, form part of the clamp portion of FIG. 7A. As seen in FIGS. 12A, 12B, 12C, 12D and 12E, fifth parts 310 each include a generally planar element 450 having a top surface 452. A first pin socket 454 is provided adjacent a corner 456 of planar element 450 and a second pin socket 458 is provided adjacent an opposite corner 460. A bottom surface 462 of generally planar element 450 is inclined such that planar element 450 has decreasing thickness from an outer facing edge 464 to an inner facing edge 466 thereof.

Reference is now made specifically to FIGS. 13A, 13B, 13C, 13D and 13E, which are each a simplified illustration of sixth part 312, two of which are arranged on opposite sides of the clamp portion of FIG. 7A with respect to axis 284. As seen in FIGS. 13A, 13B, 13C, 13D and 13E, sixth part 312 includes a generally planar element 470 having a top surface 472 defining an elongate socket 474 extending generally perpendicularly to longitudinal axis 284. First and second elongate guiding walls 476 and 478 extend perpendicularly to top surface 472 along opposite sides of elongate socket 474 to define a converging top surface tooth directing region 480 adjacent socket 474. A first pin socket 482 is defined at a first corner 484 of top surface 472 and a second pin socket 486 is defined at an adjacent corner 488 of top surface 472 and is somewhat recessed with respect to top surface 472. An overlappable surface portion 490 of top surface 472 extends below corner 488 for providing radiant energy shielding when the clamp portion is partially open. One or both edges 492 and 494 of generally planar element 470 may define a relative motion limiter with respect to an adjacent part 310.

Reference is now made specifically to FIGS. 14A, 14B, 14C, 14D and 14E, which are each a simplified illustration of seventh part 314 of the clamp portion of FIG. 7A. As seen in FIGS. 14A, 14B, 14C, 14D and 14E, seventh part 314 includes a generally planar element 500, having bifurcated pin 315, extending generally perpendicularly from a top surface 502 thereof, adjacent a rounded corner 504. Adjacent and spaced from bifurcated pin 315, a motion limiter 506 extends perpendicularly to top surface 502. Opposite motion limiter 506, a pin socket 508 extends outwardly from generally planar element 500 and is somewhat recessed with respect to top surface 502. On a bottom surface 510 of generally planar element 500 there is defined an inclined portion 512, which has decreasing thickness from an outer facing edge 514 to an inner facing edge 516.

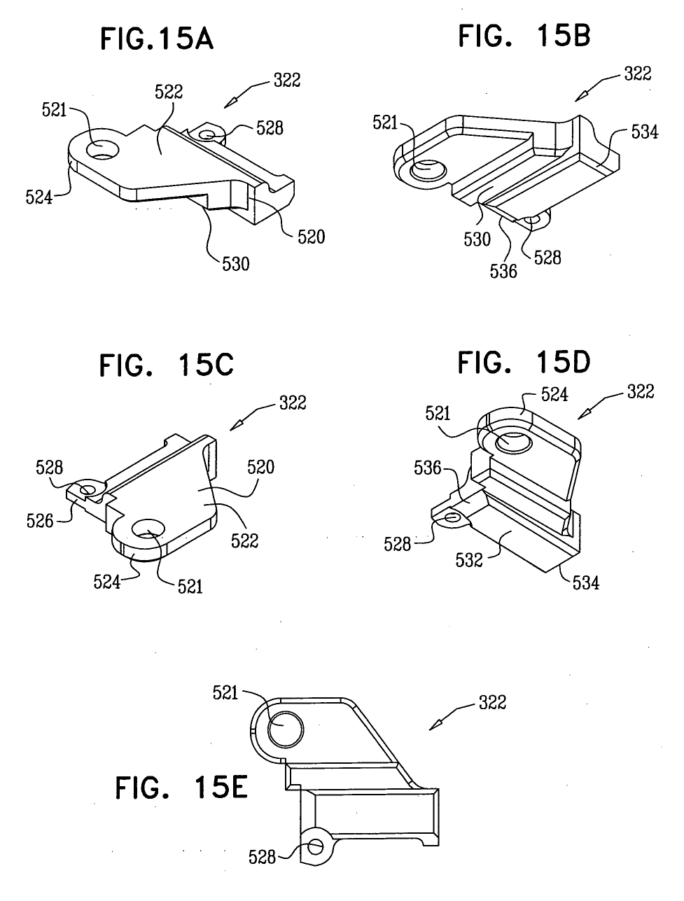

Reference is now made specifically to FIGS. 15A, 15B, 15C, 15D and 15E, which are each a simplified illustration of eighth part 322 of the clamp portion of FIG. 7A. As seen in FIGS. 15A, 15B, 15C, 15D and 15E, eighth part 322 includes a generally planar element 520 having a pin socket 521 formed in a top surface 522 thereof adjacent a rounded corner 524. At a corner 526 opposite corner 524, a pin socket 528 extends outwardly from generally planar element 520 and is somewhat recessed with respect to top surface 522. On a bottom surface 530 of generally planar element 520 there is defined an inclined portion 532, which has decreasing thickness from an outer facing edge 534 to an inner facing edge 536.

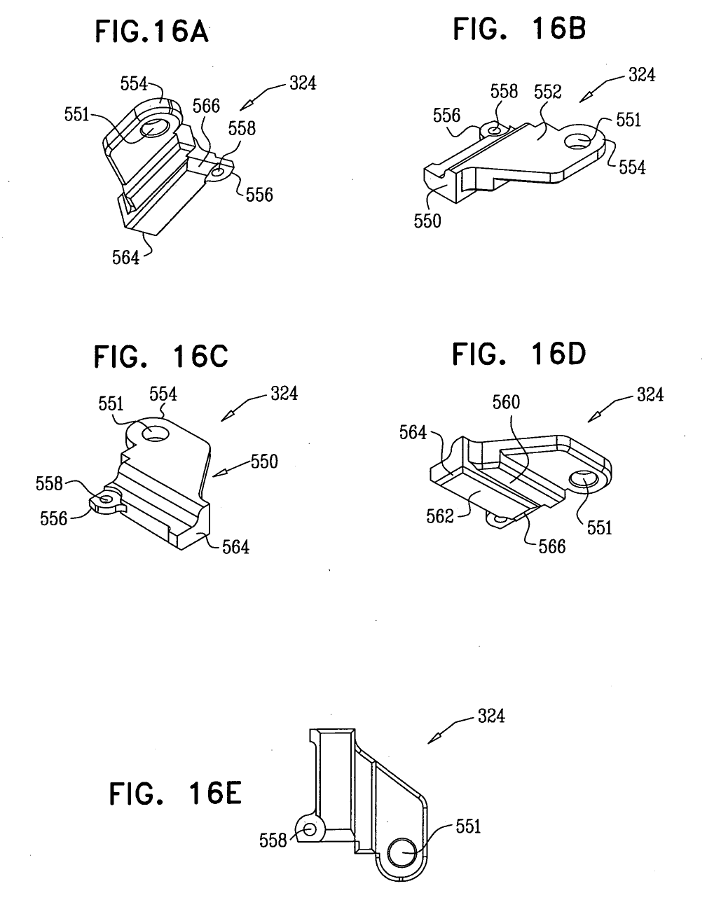

Reference is now made specifically to FIGS. 16A, 16B, 16C, 16D and 16E, which are each a simplified illustration of ninth part 324 of the clamp portion of FIG. 7A. As seen in FIGS. 16A, 16B, 16C, 16D and 16E, ninth part 324 includes a generally planar element 550 having a pin socket 551 formed in a top surface 552 thereof adjacent a rounded corner 554. At a corner 556 opposite corner 554, a pin socket 558 extends outwardly from generally planar element 550 and is somewhat recessed with respect to top surface 552. On a bottom surface 560 of generally planar element 550 there is defined an inclined portion 562, which has decreasing thickness from an outer facing edge 564 to an inner facing edge 566.

Turning now to FIG. 7B, it is seen that an alternative embodiment of clamp portion 282, here designated clamp portion 582, is comprised of a multiplicity of parts, most of which are pivotably joined together by pins 600. Clamp portion 582 is generally symmetric about a longitudinal axis 584, but, as will be appreciated, is not precisely symmetric about axis 584. As seen in FIG. 7B, the clamp portion includes, at a first side thereof, with respect to axis 284, a first part 302 having an integrally formed bifurcated pin 303, a second part 304, a pair of adjacent third parts 606, a fourth part 608, a pair of adjacent fifth parts 610, a sixth part 312 and a seventh part 314, having an integrally formed pin 315. The clamp portion 582 also includes, at a second side thereof, with respect to axis 584, an eighth part 322, a second part 304, a pair of adjacent third parts 606, a fourth part 608, a pair of adjacent fifth parts 610, a sixth part 312 and a ninth part 324.

Third parts 606, fourth parts 608 and fifth parts 610 are similar to corresponding third parts 306, fourth parts 308 and fifth parts 310 (FIG. 7A) other than in that they each include a spray nozzle for gas driven spraying of tissue bonding enhancement material, a container for tissue bonding enhancement material upstream of the spray nozzle and a pressurized gas inlet upstream of the container to which is coupled a gas hose 611, coupled in turn to a pressurized gas manifold 612, one or more of which manifolds may be provided.

Reference is now made to FIGS. 17A, 17B, 17C, 17D, 17E and 17F, which are each a simplified illustration of third part 606 of the clamp portion of FIG. 7B, four of which, arranged in side by side pairs on opposite sides of axis 584, form part of the clamp portion of FIG. 7B. As seen in FIGS. 17A, 17B, 17C, 17D, 17E and 17F, third parts 606 each include a generally planar element 613 having a top surface 614. A first pin socket 615 is provided adjacent a corner 616 of planar element 613 and a second pin socket 618 is provided adjacent an opposite corner 620. A bottom surface 622 of generally planar element 613 is inclined such that planar element 613 has decreasing thickness from an outer facing edge 624 to an inner facing edge 626 thereof.

As distinguished from third part 306 (FIGS. 7A and 10A-10D), third part 606 includes a bonding enhancement material dispenser 640 which includes a container portion 641 which contains bonding enhancement material 642 and an outlet conduit 644 which communicates with a nozzle 645 at inner facing edge 626. Container portion 641 includes an inlet connector end 646 which is adapted to be connected to a source of pressurized gas, preferably via gas hose 611 and manifold 612 (FIG. 7B), to selectively cause a spray discharge of the bonding enhancement material 642, as shown schematically at reference numeral 648.

Reference is now made to FIGS. 18A, 18B, 18C, 18D, 18E and 18F, which are each a simplified illustration of fourth part 608, two of which are arranged on opposite sides of the clamp portion of FIG. 7B with respect to axis 584. As seen in FIGS. 18A, 18B, 18C, 18D, 18E and 18F, fourth part 608 includes a generally planar element 650 having a top surface 652 defining an elongate socket 654 extending generally perpendicularly to longitudinal axis 584. First and second elongate guiding walls 656 and 658 extend perpendicularly to top surface 652 along opposite sides of elongate socket 654 to define a converging top surface tooth directing region 670 adjacent socket 654. A first pin socket 672 is defined at a first corner 674 of top surface 652 and a second pin socket 676 is defined at an adjacent corner 678 of top surface 652. Overlappable surface portions 680 and 682 of top surface 652 extend below respective corners 674 and 678 for providing radiant energy shielding when the clamp portion is partially open. It is a particular feature of the structure of fourth part 608 that an inner facing end surface 684 of elongate socket 654 is undercut.

As distinguished from fourth part 308 (FIGS. 7A and 11A-11F), fourth part 608 includes a bonding enhancement material dispenser 690 which includes a container portion 691 which contains bonding enhancement material 692 and an outlet conduit 694 which communicates with a nozzle 695 at an inner facing edge of part 608. Container portion 691 includes a connector end 696 which is adapted to be connected to a source of pressurized gas, preferably via gas hose 611 and manifold 612 (FIG. 7B), so as to selectively cause a spray discharge of the bonding enhancement material 692, as shown schematically at reference numeral 698.

Reference is now made to FIGS. 19A, 19B, 19C, 19D, 19E and 19F, which are each a simplified illustration of fifth part 610 of the clamp portion of FIG. 7B, four of which, arranged in side by side pairs on opposite sides of axis 584, form part of the clamp portion of FIG. 7B. As seen in FIGS. 19A, 19B, 19C, 19D, 19E and 19F, fifth parts 610 each include a generally planar element 700 having a top surface 702. A first pin socket 704 is provided adjacent a corner 706 of planar element 700 and a second pin socket 708 is provided adjacent an opposite corner 710. A bottom surface 712 of generally planar element 700 is inclined such that planar element 700 has decreasing thickness from an outer facing edge 714 to an inner facing edge 716 thereof.

As distinguished from fifth part 310 (FIGS. 7A and 12A-12E), fifth part 610 includes a bonding enhancement material dispenser 720 which includes a container portion 721 which contains bonding enhancement material 722 and an outlet conduit 724 which communicates with a nozzle 725 at an inner facing edge of part 610. Container portion 721 includes a connector end 726 which is adapted to be connected to a source of pressurized gas, preferably via air hose 611 and manifold 612 (FIG. 7B), so as to selectively cause a spray discharge of the bonding enhancement material 722, as shown schematically at reference numeral 728.

Reference is now made to FIGS. 20A and 20B, which are each a simplified pictorial illustration of a clamp engagement element, such as clamp engagement elements 252 and 254, forming part of the system of FIGS. 4A-4D and 5. As seen in FIGS. 20A and 20B, the clamp engagement element preferably comprises a generally rectangular planar portion 802, having first, second and third protrusions 804, 806 and 808 extending generally perpendicular thereto.

Protrusions 804 and 808 are mirror images of each other and each include a planar portion 810 having a generally inclined top edge portion 812 extending to a generally rounded corner 814 and therefrom as a slightly inclined bottom edge portion 816, extending to a point 818. An undercut internal edge portion 820 extends from point 818 to an internal edge junction 822 and an inclined lower edge portion 824 extends from junction 822 to an inclined bottom edge of planar portion 802. A generally triangular reinforcement portion 826 extends generally perpendicularly to planar portion 810 in congruity with lower edge portion 824.

Protrusion 806 includes a planar portion 830 having a generally inclined top edge portion 832 extending from a location just below an aperture 834 formed in planar portion 802 to a point 836 and therefrom as a bottom edge portion 838, extending generally perpendicularly to planar portion 802 to an external edge junction 840. An inclined lower edge portion 842 extends from junction 840 to an inclined bottom edge of planar portion 802.

Reference is now made to FIGS. 21A, 21B, 21C and 21D, which are each a simplified illustration of a clamp manipulation subassembly, such as clamp manipulation subassembly 256, forming part of the system of FIGS. 4A-4D and 5. The clamp manipulation subassembly preferably comprises a chassis, indicated generally by reference numeral 850 and which includes a pair of generally parallel spaced chassis frame elements 852 and 854, which are generally identical mirror images of each other, other than as specifically described hereinbelow. Chassis frame elements 852 and 854 are maintained in desired mutually spaced parallel orientation by spacer rods 856 and 858, which are engaged by fasteners 860.

First and second generally axially displaceable clamp engagement element displacers 862 and 864 are arranged for selectable axial displacement towards and away from each other with respect to a displacement axis 866, which extends generally perpendicular to the longitudinal extent of generally parallel spaced chassis frame elements 852 and 854. Displacers 862 and 864 are both threadably mounted by means of couplings 867 onto a pair of rotatable threaded axles 868 and 870, each of which is formed with threading having opposite sense along different portions 872 and 874 of the length thereof. Axles 868 and 870 are mounted onto respective frame elements 852 and 854 by means of bearings 876 and are each provided with a drive gear, here designated respectively by reference numerals 878 and 880.

Drive gears 878 and 880 are respectively driven by gears 882 and 884, which are preferably mounted on respective drive shafts 886 and 888 of electric motors 890 and 892, mounted on frame element 852.

It is appreciated that the mounting arrangement of displacers 862 and 864 is such that operation of electric motors 890 and 892, in a first direction of rotation, causes the displacers 862 and 864 simultaneously to move towards each other along axles 868 and 870, parallel to axis 866, and that operation of electric motors 890 and 892, in a second direction of rotation, causes the displacers 862 and 864 simultaneously to move away from each other along axles 868 and 870, parallel to axis 866.

Couplings 867 preferably allow limited non-axial displacement of individual displacers 862 and 864 with respect to each other and to axis 866.

Mounted on respective displacers 862 and 864 are respective spring-loaded, manually operable engagement assemblies 898 for selectable engagement of clamp engagement elements 252 and 254. Each of assemblies 898 includes an axially displaceable finger engagement portion 899 fixedly coupled to an engagement plate 900 having formed thereon a protrusion 902 which is adapted to engage aperture 834 (FIGS. 20A and 20B) formed in a clamp engagement element 252 or 254. When finger engagement portion 899 is not inwardly depressed by a user, engagement plate 900 is urged by springs 904 against a displacer 862 or 864 thus securely retaining a clamp engagement element 252 or 254 therebetween.

In accordance with a preferred embodiment of the invention a load cell 910 is mounted on each of displacers 862 and 864 adjacent respective engagement plates 900 so as to sense the force exerted by the displacers 862 and 864 on clamp engagement elements 252 and 254 respectively and thus sense the tensile force exerted on the tissue at the seam being bonded.

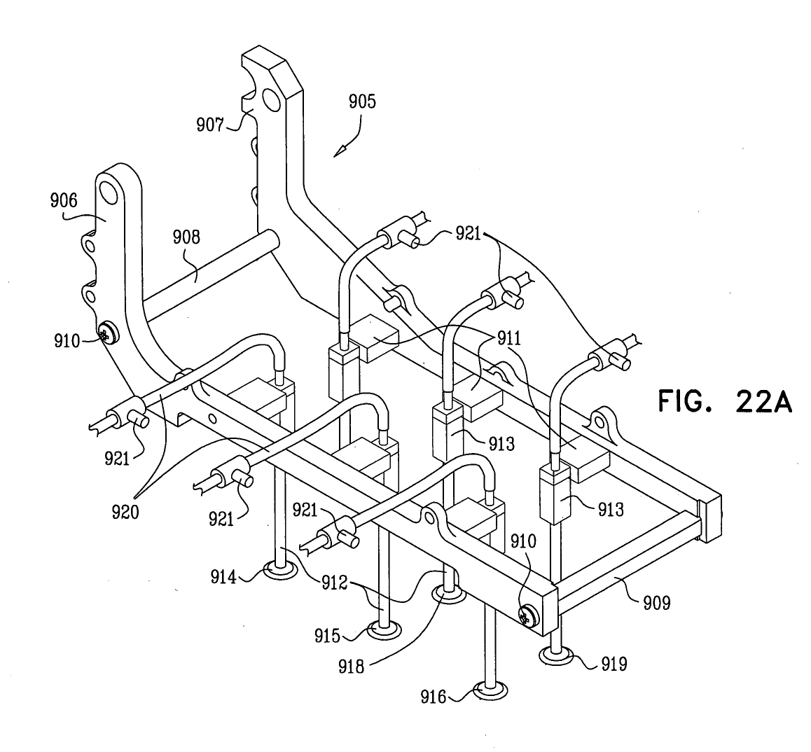

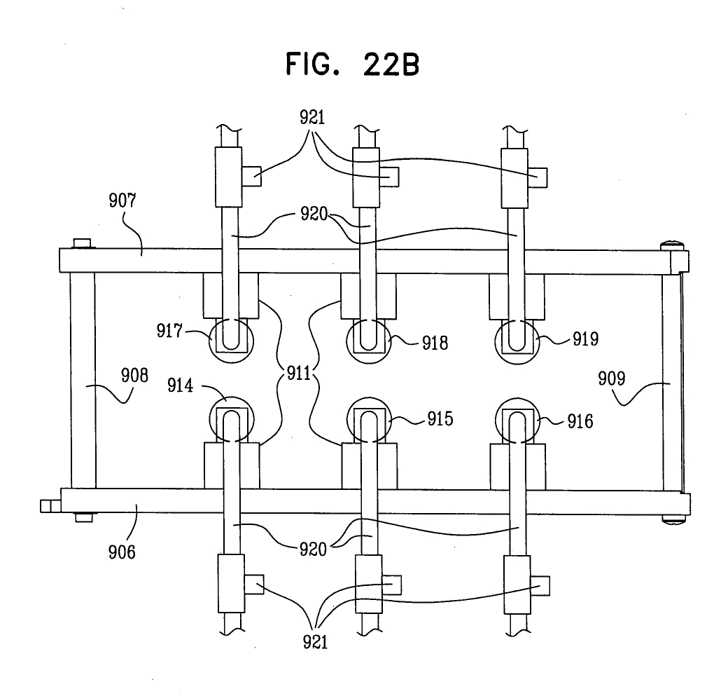

Reference is now made to FIGS. 22A and 22B, which are each a simplified illustration of a tissue manipulation subassembly forming part of an alternative embodiment of the system of FIGS. 4A-4D and 5. The tissue manipulation subassembly preferably comprises a chassis, indicated generally by reference numeral 905 and which includes a pair of generally parallel spaced chassis frame elements 906 and 907, which are generally identical mirror images of each other, other than as specifically described hereinbelow. Chassis frame elements 906 and 907 are maintained in desired mutually spaced parallel orientation by spacer rods 908 and 909, which are engaged by fasteners 910.

A plurality of individually controllable linear motors 911, preferably at least three in number, are mounted on each of chassis frame elements 906 and 907. Each linear motor 911 supports an arm 912 that preferably includes a solenoid 913, which provides controllable vertical displacement of the arm 912. At a lower end of arm 912 there is preferably provided a tissue engagement head such as a vacuum cup, needle array or any other suitable non-sliding engagement assembly. In the illustrated embodiment, a total of 6 vacuum cups are provided, designated by respective reference numerals 914, 915, 916, 917, 918 and 919.

In the illustrated embodiment of the invention, vacuum cups 914-919 are provided along with associated vacuum conduits 920, having individually controllable vacuum valves 921. The linear motors 911, the solenoids 913 and the vacuum valves 921 preferably are all controlled by computerized control unit 210 (FIG. 3).

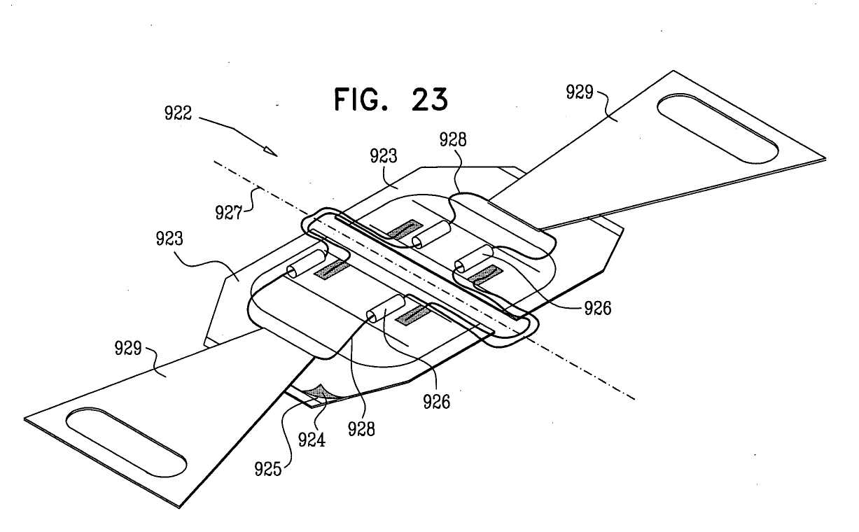

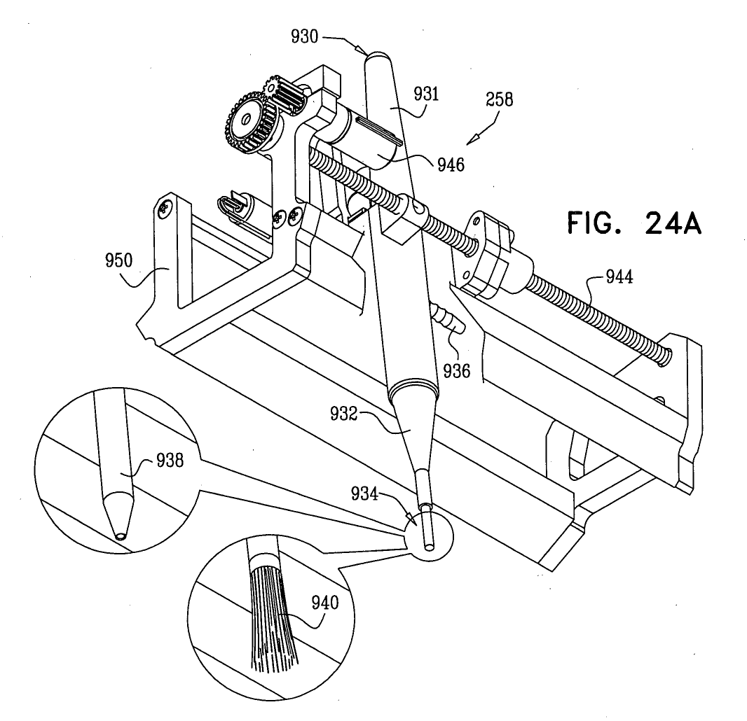

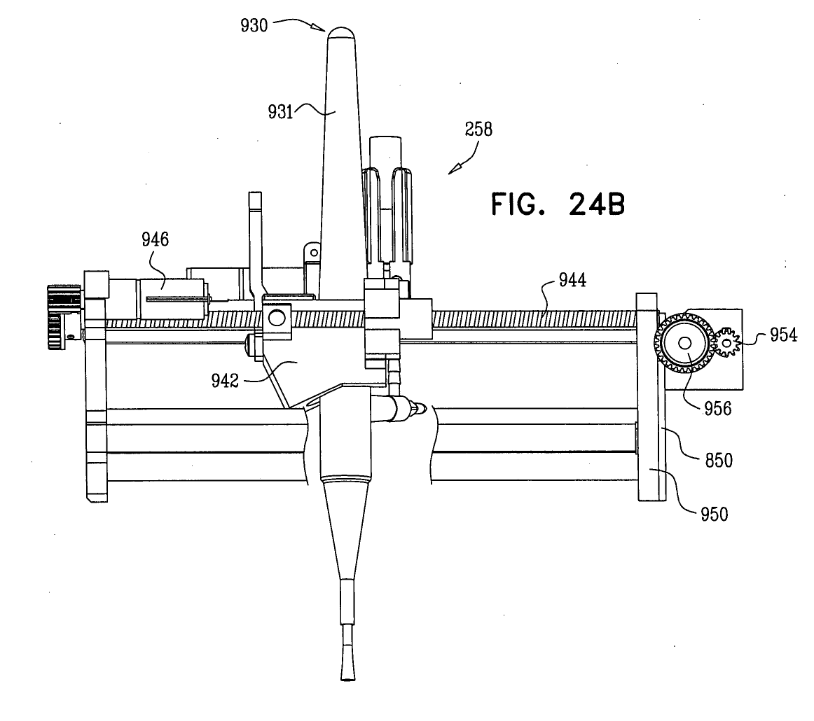

Reference is now made to FIG. 23, which is a simplified partially exploded view illustration of a bandage assembly 922 forming part of an alternative embodiment of the system of FIG. 3 and useful with the tissue manipulation subassembly of FIGS. 22A and 22B.

As seen in FIG. 23, the bandage assembly 922 comprises a pair of side bandage portions 923, each having an adhesive surface 924, for attachment to a patient's body, and a release layer 925. Fixed to each bandage portion 923 is a plurality of conduits 926 extending generally perpendicularly to a longitudinal axis 927. A plurality of elongate elements 928 each extends through a corresponding one of the plurality of conduits 926. One end of each of the elongate elements 928 extending through conduits 926 on each side bandage portion 923 is connected to a common pull-tab 929. The opposite end of each of the elongate elements 928 extending through conduits 924 on each side bandage portion 923 is fixed to the opposite side bandage portion 923.

Reference is now made to FIGS. 24A and 24B, which are each a simplified illustration of a tissue bonding enhancement material dispenser subassembly, such as tissue bonding enhancement material dispenser subassembly 258, forming part of the system of FIGS. 4A-4D and 5. The tissue bonding enhancement material dispenser subassembly 258 comprises a material supply assembly 930 including generally tubular container 931 which is coupled via a valve 932 to a dispenser assembly 934. In one embodiment of the invention, the dispenser assembly comprises an airbrush and includes a pressurized gas inlet 936 and an outlet nozzle 938. In an alternative embodiment of the invention, the dispenser assembly does not employ pressurized gas and rather employs gravity feed of the bonding enhancement material via a brush nozzle 940.

The material supply assembly 930 is preferably mounted on a X-Y displaceable platform 942 which is threadably mounted onto a screw displacer 944, which is in turn rotatably driven by an electric motor 946 for displacement along an X axis, perpendicular to axis 866 (FIG. 21D).

The material supply assembly 930, platform 942, screw displacer 944 and electric motor 946 are all mounted onto a carriage 950 for displacement along a Y axis, parallel to axis 866 (FIG. 21D). Displacement of carriage 950 along the Y axis is provided by an electric motor 952 (FIG. 5) which is preferably mounted onto chassis 850 of clamp manipulation subassembly 256 (FIG. 5) and which drives, via gears 954 and 956, a threaded shaft 958 (FIG. 5), which is also mounted onto chassis 850 by bearings 960 (FIG. 5). Carriage 950 is threadably mounted onto threaded shaft 958 for displacement responsive to rotation of threaded shaft 958.

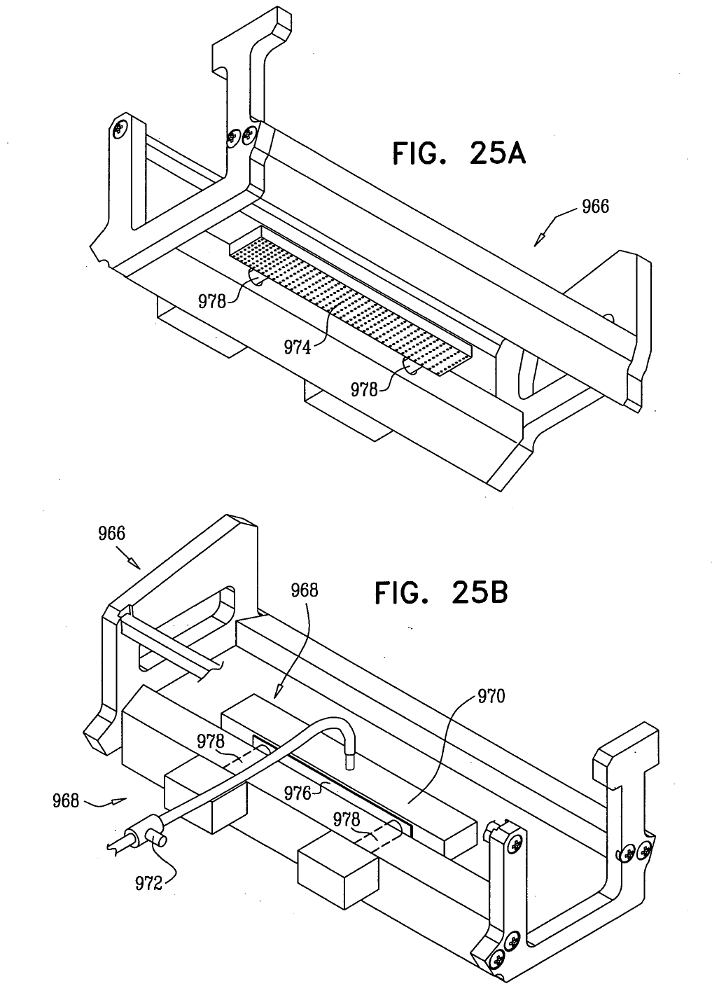

Reference is now made to FIGS. 25A and 25B, which are each a simplified pictorial illustration of an alternative embodiment of a tissue bonding enhancement material dispenser subassembly forming part of the system of FIGS. 4A-4D and 5. The tissue bonding enhancement material dispenser subassembly, here designated by reference numeral 966, comprises a material supply assembly 968 including a container 970 which is coupled via a valve 972 to a source of pressurized gas (not shown). In this embodiment of the invention, container 970 has a downward facing wall formed with a multiplicity of perforations 974 through which tissue bonding enhancement material is forced under appropriate gas pressure.

The container 970 is preferably mounted on a linearly displaceable platform 976 which is mounted onto solenoid driven displacers 978 for displacement along an Y axis, parallel to axis 866 (FIG. 21D).

The platform 976 and displacers 978 are all mounted onto chassis 850 of clamp manipulation subassembly 256 (FIGS. 21A-21D).

Reference is now made to FIGS. 26A, 26B and 26C, which are each a simplified pictorial illustration of an energy directing subassembly, such as energy directing subassembly 260, forming part of the system of FIGS. 4A-4D and 5. Energy directing subassembly 260, to which is coupled flexible optical fiber 208 (FIG. 3) or alternatively an articulated arm mirror assembly (not shown) is fixedly mounted onto chassis 850 of clamp manipulation subassembly 256 (FIGS. 21A-21D) by means of supporting legs 980 which support a platform 982.

Mounted onto platform 982 is an X-Y optical scanner 984, such as a Model 6200H Galvanometer Optical Scanner, commercially available from CAMBRIDGE TECHNOLOGY, INC, 25 Hartwell Ave., Lexington, Mass. 02421, USA, which receives an output of laser 206 (FIG. 3) via optical fiber 208 or an articulated arm mirror assembly (not shown) and provides any suitable scanning pattern. Also mounted onto platform 982 is an IR temperature sensor 990, such as a model IN 510-N digital pyrometer, commercially available from IMPAC Infrared GmbH of Frankfurt, Germany, for sensing temperature of the tissue or the bonding material at the seam. Additionally mounted on platform 982 there is preferably provided a video camera 992 for viewing the bonding operation and thus enabling control and/or monitoring thereof.

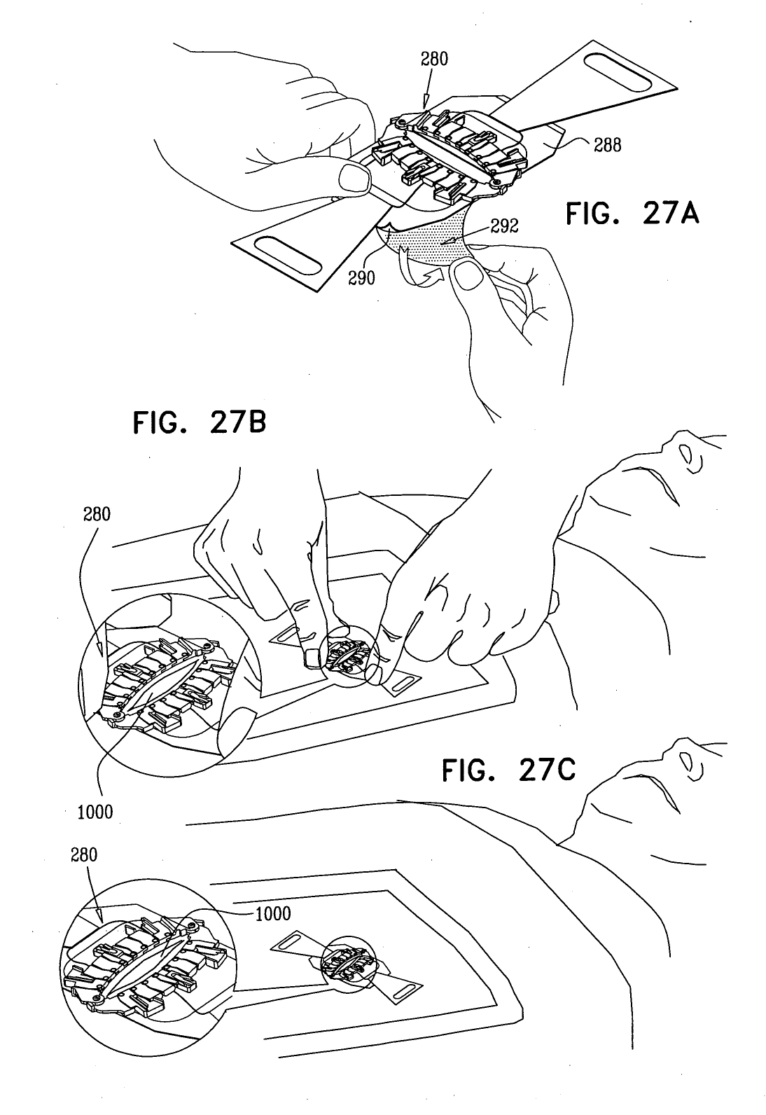

Reference is now made to FIGS. 27A, 27B and 27C, which are each a simplified pictorial illustration of a stage in the operation of the system of FIGS. 1A, 1B and 3-21D and 24A-26C wherein the clamp assembly 280 (FIG. 6) is mounted onto a patient in propinquity to a cut to be bonded. FIG. 27A shows removal of release layers 292 from adhesive surfaces 290 of side bandage portions 288. FIG. 27B shows placement of the clamp assembly 280 over a cut 1000 with adhesive surfaces 290 of side bandage portions 288 being pressed into adhesive engagement with the patient's skin adjacent cut 1000. FIG. 27C shows the clamp assembly 280 retained in place over cut 1000 by this adhesive engagement.

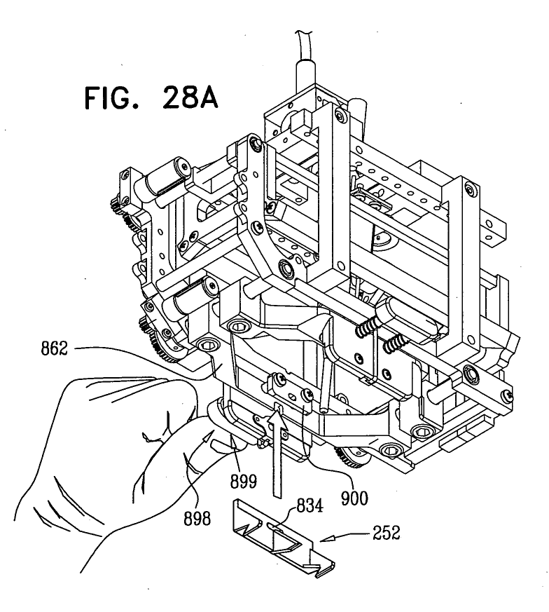

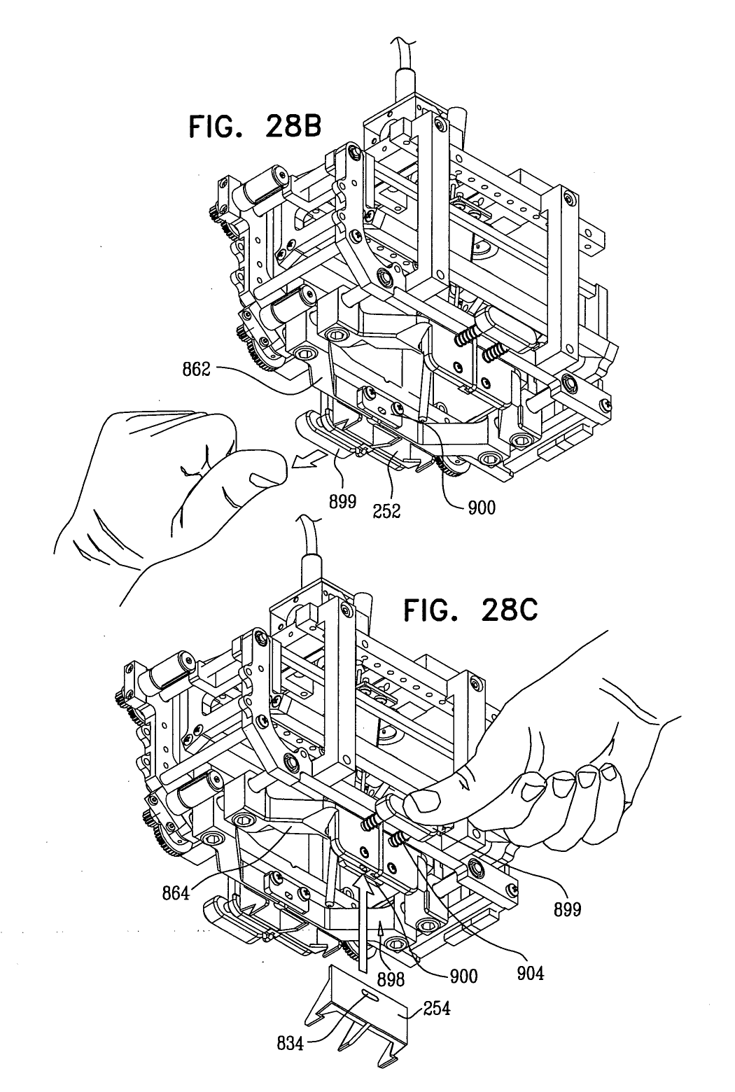

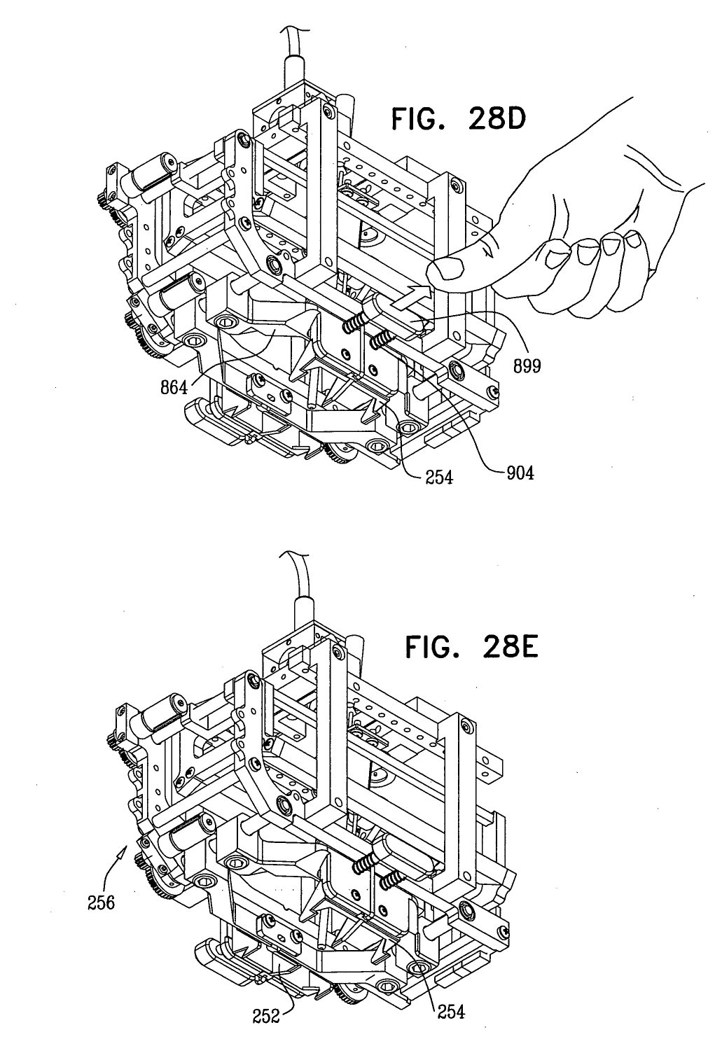

Reference is now made to FIGS. 28A, 28B, 28C, 28D and 28E which are each a simplified pictorial illustration of a further stage in the operation of the system of FIGS. 1A, 1B and 3-21D and 24A-26C wherein clamp engagement elements 252 and 254 (FIGS. 20A & 20B) are retained in the clamp manipulation subassembly 256 (FIGS. 21A-21D).

FIG. 28A shows a user pressing on one of finger engagement portions 899 forming part of a corresponding spring-loaded, manually operable engagement assembly 898 for selectable engagement of clamp engagement element 252. Pressing of finger engagement portion 899 causes engagement plate 900, having formed thereon protrusion 902 (FIG. 21D) which is adapted to engage aperture 834 formed in clamp engagement element 252, to be displaced against the urging of spring 904 (FIG. 21D) away from displacer 862. This allows insertion of clamp engagement element 252 between displacer 862 and engagement plate 900.

FIG. 28B shows release of finger engagement portion 899 by the user following insertion of clamp engagement element 252 between engagement plate 900 and displacer 862, thus allowing spring 904 (FIG. 21D) to urge engagement plate 900 against displacer 862, thus securely retaining clamp engagement element 252 therebetween.

FIG. 28C shows a user pressing on one of finger engagement portions 899 forming part of a corresponding spring-loaded, manually operable engagement assembly 898 for selectable engagement of clamp engagement element 254. Pressing of finger engagement portion 899 causes engagement plate 900, having formed thereon protrusion 902 (FIG. 21D) which is adapted to engage aperture 834 (FIGS. 20A and 20B) formed in clamp engagement element 254, to be displaced against the urging of spring 904 away from displacer 864. This allows insertion of clamp engagement element 254 between displacer 864 and engagement plate 900.

FIG. 28D shows release of finger engagement portion 899 by the user following insertion of clamp engagement element 254 between engagement plate 900 and displacer 864, thus allowing spring 904 to urgent engagement plate 900 against displacer 864 thus securely retaining clamp engagement element 254 therebetween.

FIG. 28E shows the clamp manipulation subassembly 256 (FIGS. 21A-21D) having clamp engagement elements 252 and 254 securely mounted thereon.

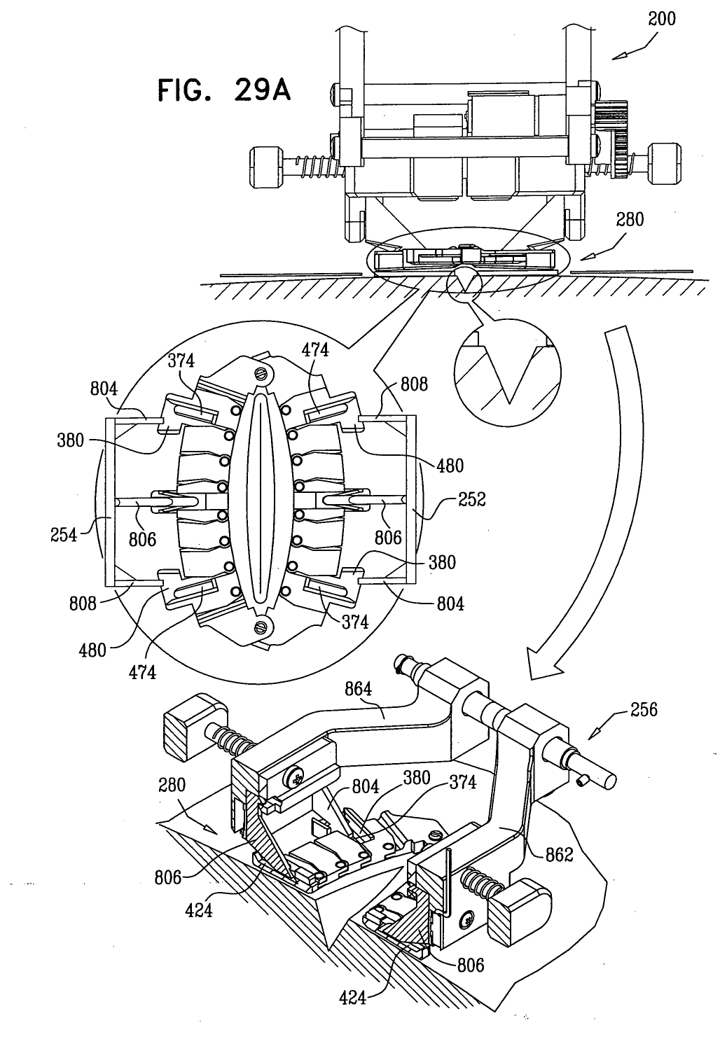

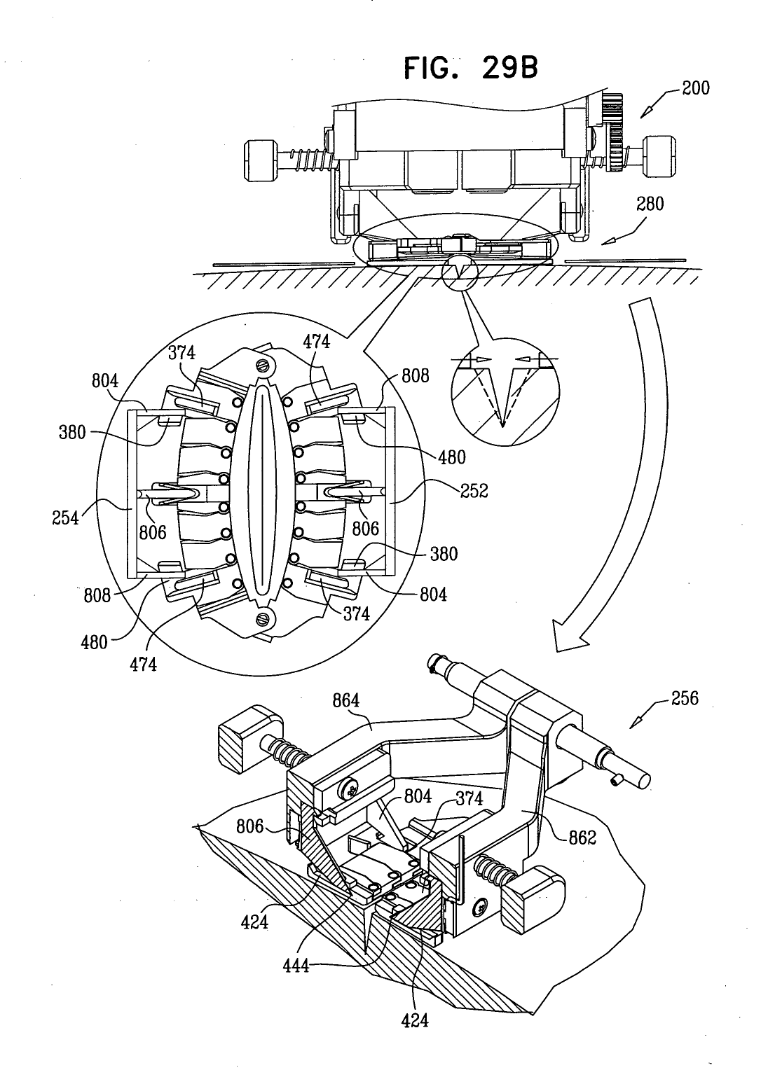

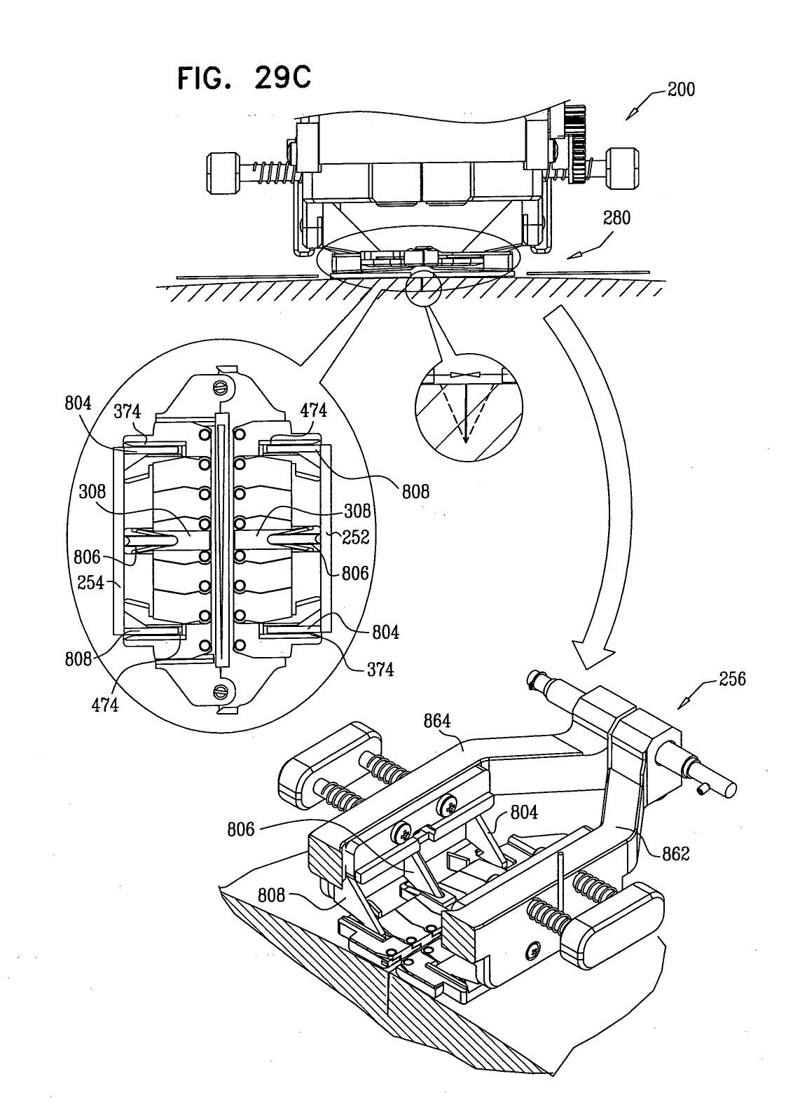

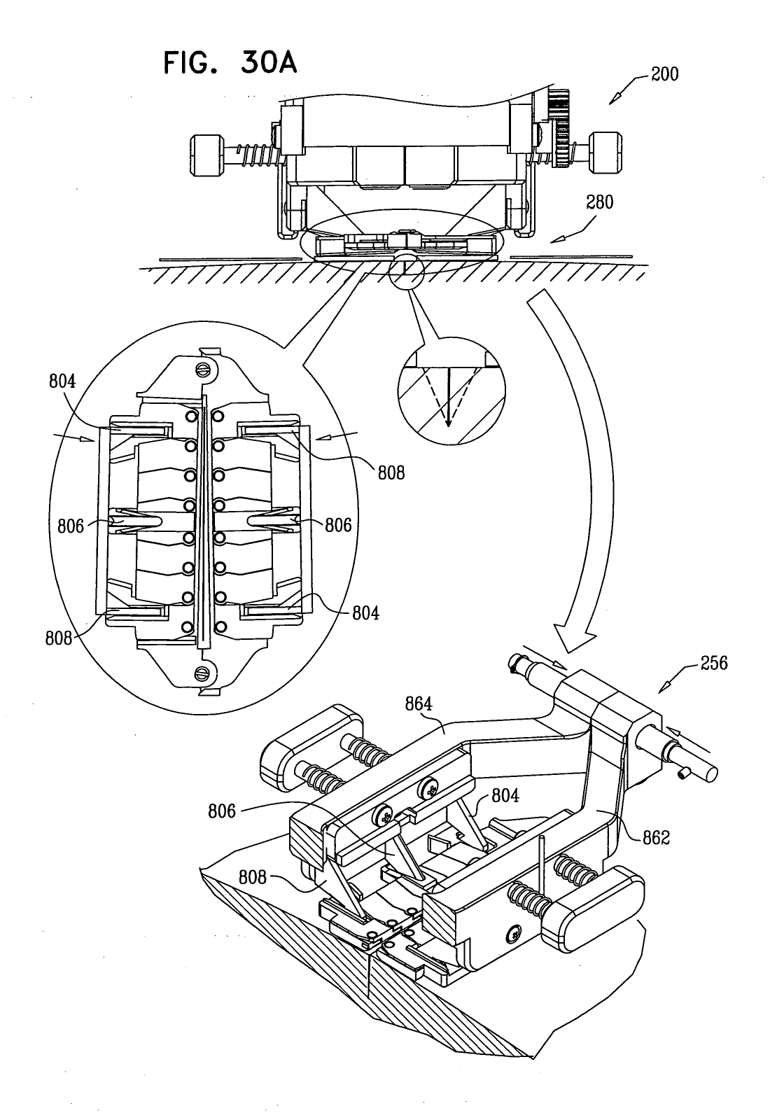

Reference is now made to FIGS. 29A, 29B and 29C, which are each a simplified pictorial illustration of a further stage in the operation of the system of FIGS. 1A, 1B and 3-21D and 24A-26C wherein clamp engagement elements (FIGS. 20A & 20B), while retained in the clamp manipulation subassembly (FIGS. 21A-21D), are caused to engage the clamp assembly 280 and are displaced by the clamp manipulation subassembly (FIGS. 21A-21D) causing initial closing displacement of the clamp assembly 280 and thus of cut 1000, prior to application of energy or of bonding enhancement material to the cut 1000.

FIG. 29A shows placement of the patient mounted portion 200 of the system onto clamp assembly 280 by an operator. The operator orients the patient mounted portion 200 such that protrusions 806 of respective clamp engagement elements 252 and 254, retained by clamp manipulation subassembly 256 thereof, lie in respective elongate sockets 424 (FIGS. 11A-11F) of clamp assembly 280. Orientation of patient mounting portion 200 such that protrusions 806 lie in respective elongate sockets 424 causes protrusions 804 and 808 of respective clamp engagement elements 252 and 254 to lie in corresponding converging top surface tooth directing region 380 adjacent elongate socket 374 (FIGS. 9A-9D) and in corresponding converging top surface tooth directing region 480 adjacent elongate socket 474 (FIGS. 13A-13E), respectively.

FIG. 29B shows initial closing operation of clamp manipulation subassembly 256 wherein clamp engagement elements 252 and 254 are brought slightly closer together such that points 836 (FIG. 20B) of respective protrusions 806 each engage undercut inner facing end surfaces 444 of respective elongate sockets 424. This initial closing operation is produced by corresponding relative displacement of clamp engagement element displacers 862 and 864, driven by electric motors 890 and 892 (FIG. 21D), respectively. Operation of patient mounting portion 200 such that protrusions 806 engage undercut inner facing end surfaces 444 causes protrusions 804 and 808 of respective clamp engagement elements 252 and 254 to be displaced in corresponding converging top surface tooth directing region 380 towards adjacent elongate socket 374 (FIGS. 9A-9D) and in corresponding converging top surface tooth directing region 480 towards adjacent elongate socket 474 (FIGS. 13A-13E), respectively.

FIG. 29C shows further closing operation of clamp manipulation subassembly 256 wherein clamp engagement elements 252 and 254 are brought even closer together such that points 836 (FIG. 20B) of respective protrusions 806, which engage undercut inner facing end surfaces 444 of respective elongate sockets 424, force corresponding fourth parts 308 of clamp assembly 280 towards each other. This further closing operation is also produced by corresponding relative displacement of clamp engagement element displacers 862 and 864, driven by electric motors 890 and 892, respectively. Operation of patient mounting portion 200 such that protrusions 806, which engage undercut inner facing end surfaces 444, displace fourth parts 308 of clamp assembly 280 also causes protrusions 804 and 808 of respective clamp engagement elements 252 and 254 to be located in respective elongate sockets 374 (FIGS. 9A-9D) and 474 (FIGS. 13A-13E).

It is appreciated that the orientation of clamp assembly 280 at this stage is generally along a straight line, defined by the longitudinal axis of the cut 1000, as distinguished from a bowed orientation seen in FIGS. 29A and 29B. It is also appreciated that the edges of the skin along cut 1000 are correspondingly oriented along a straight line, as distinguished from the bowed orientation shown in FIGS. 27A-27C and 29A-29B. It is also appreciated that at this stage, the patient mounted portion 200 is centered over the cut 1000.

It is further appreciated that when the clamp is being closed on the cut 1000, the inclined planar portions of parts 304-312 of clamp portion 282, as seen in FIGS. 7A-19F, force the edges of the cut upwards to create enhanced cosmetic closure.

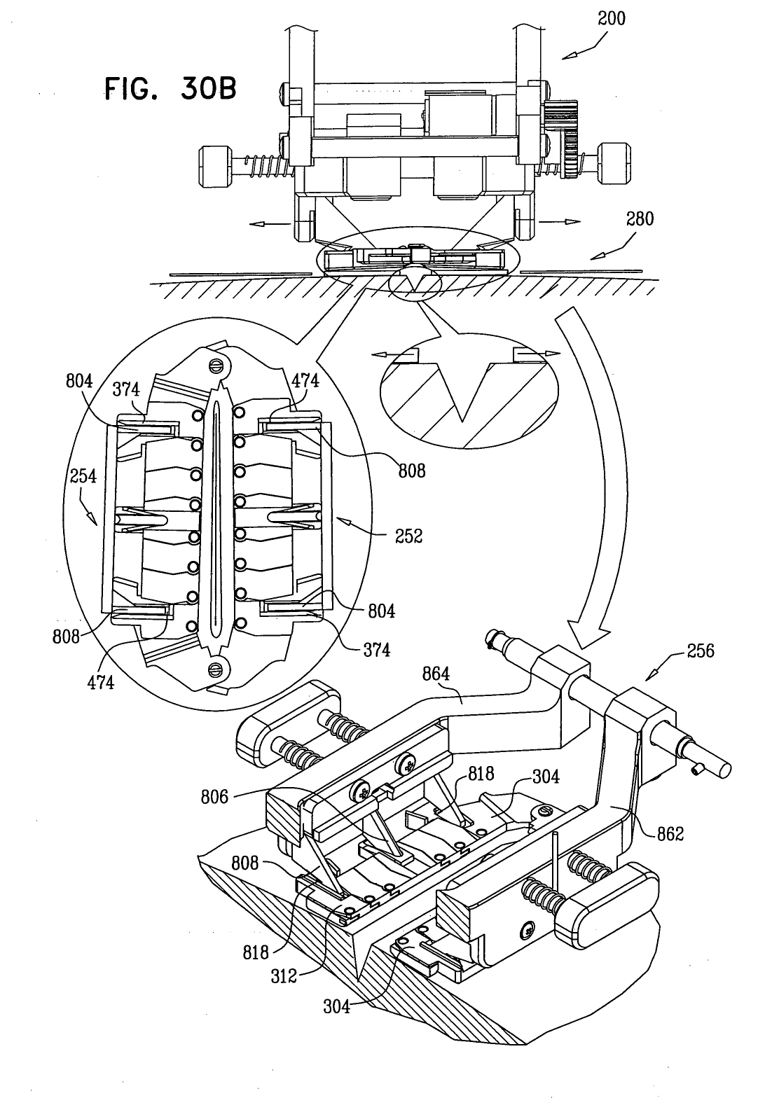

Reference is now made to FIGS. 30A and 30B, which illustrate limited non-axial mutual orientation of the edges of the cut 1000 and subsequent limited axial opening of the cut.

FIG. 30A shows limited non-axial mutual reorientation of the edges of the cut 1000 to produce an esthetically optimized closure. This is preferably achieved by individual actuation of electric motors 890 and 892 via couplings 867 (FIGS. 21A-21D), which, as noted above, allows limited non-axial displacement of individual displacers 862 and 864 with respect to each other and to axis 866.

FIG. 30B shows limited axial opening of the cut 1000 by opening operation of clamp manipulation subassembly 256 wherein clamp engagement elements 252 and 254 are displaced away from each other along an axis which is perpendicular to the longitudinal axis of the cut. It is appreciated that points 818 of respective protrusions 804 and 808 engage respective back surfaces of respective elongate sockets 374 and 474, forcing corresponding second and sixth parts 304 and 312 of clamp assembly 280 axially away from each other. This limited opening operation is produced by corresponding relative displacement of clamp engagement element displacers 862 and 864 driven by electric motors 890 and 892 (FIGS. 21A-21D) respectively.

It is appreciated that the operation of clamp manipulation subassembly 256 together with clamp engagement elements 252 and 254 and clamp assembly 280 is capable, inter alia, of reconfiguring an excision, having a bowed orientation, as an incision.

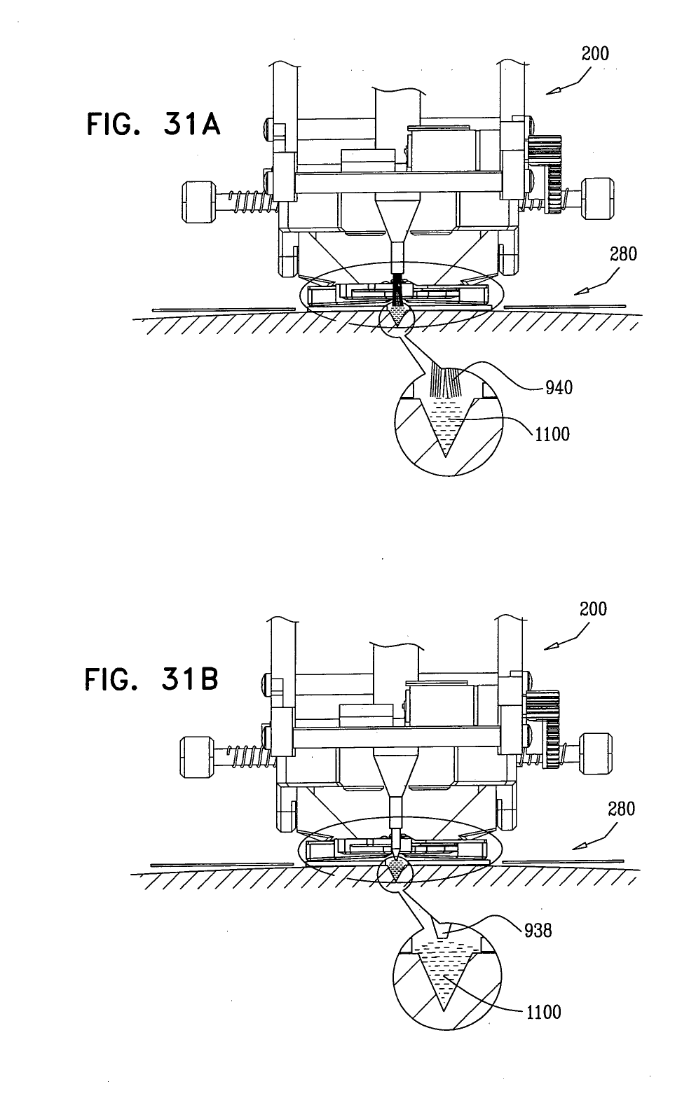

Reference is now made to FIGS. 31A & 31B, which are simplified pictorial illustrations of two alternative embodiments of a still further stage in the operation of the system of FIGS. 1A, 1B and 3-7A and 8A-16E and 20A-24B and 26A-26C, wherein bonding enhancement material is applied by a brush applicator and an air brush applicator respectively, as shown in FIGS. 24A and 24B. As seen in FIG. 31A, a bonding enhancement material 1100, such as human serum albumin or alternatively any other suitable tissue bonding enhancement material, is applied to the open cut 1000 and to surrounding skin by brush nozzle 940. FIG. 31B shows bonding enhancement material being applied to the open cut 1000 and to surrounding skin by an air brush through outlet nozzle 938.

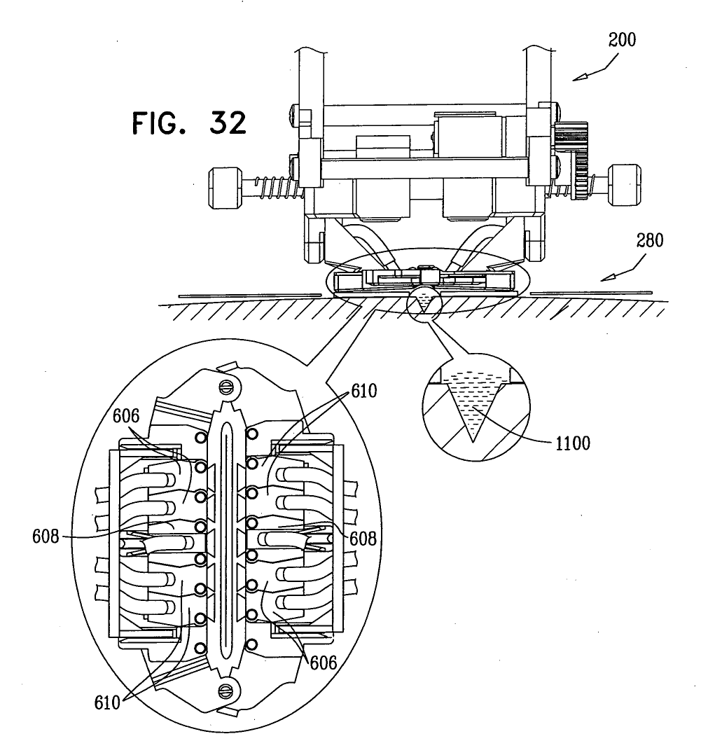

Reference is now made to FIG. 32, which is a simplified pictorial illustration of a bonding enhancement material application stage in the operation of the system of FIGS. 1A, 1B and 3-6 and 7B-9E and 13A-21D and 26A-26C, wherein bonding enhancement material is applied in accordance with yet another embodiment of the present invention employing clamp mounted applicators as shown in FIG. 7B. As seen in FIG. 32, a bonding enhancement material 1100, such as human serum albumin or alternatively any other suitable tissue bonding enhancement material, is applied to the open cut 1000 and to surrounding skin from various parts of the clamp assembly, the third, fourth and fifth parts of the clamp assembly of FIG. 7B (FIGS. 17A-19F), preferably by a gas-driven spray.

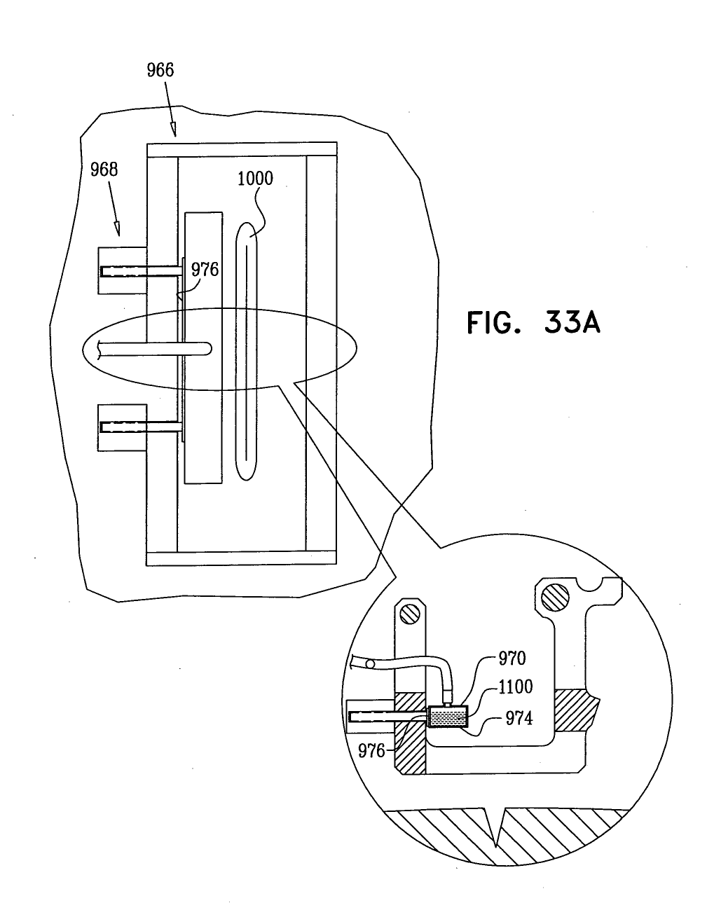

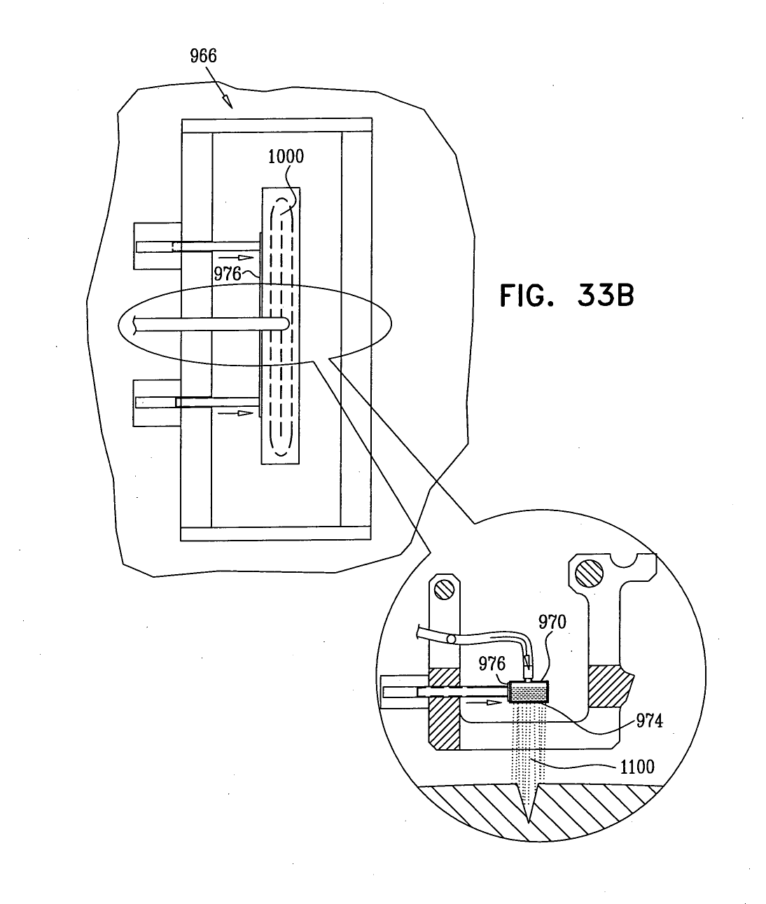

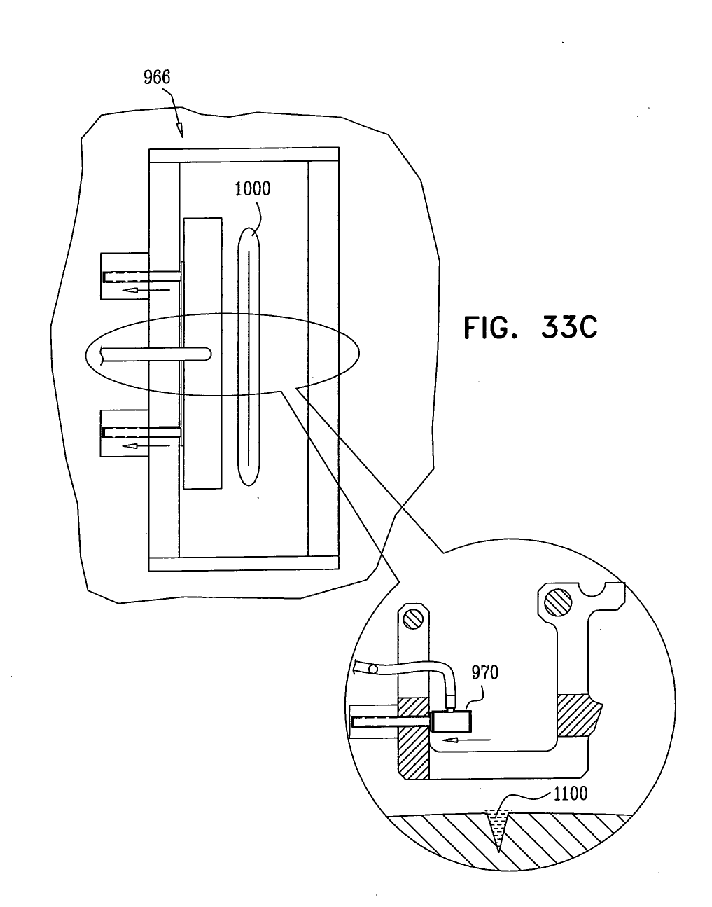

Reference is now made to FIGS. 33A-33C, which together are a simplified pictorial illustration of a bonding enhancement material application stage in the operation of the system of FIGS. 1A, 1B and 3-7A and 8A-16E and 20A-23 and 25A-26C wherein bonding enhancement material is applied in accordance with still another embodiment of the present invention employing a tissue bonding enhancement material dispenser subassembly 966, as shown in FIGS. 25A-B.

FIG. 33A shows the container 970, mounted on linearly displaceable platform 976 at a first, pre-dispensing location along the Y axis. FIG. 33B shows the container 970 at a dispensing location overlying the open cut 1000, dispensing the bonding enhancement material 1100 onto the cut via perforations 974. FIG. 33C shows the container 970 returned to the pre-dispensing location of FIG. 33A, following dispensing of the bonding enhancement material 1100 onto the open cut. It is appreciated that alternatively multiple containers 970 may be employed.

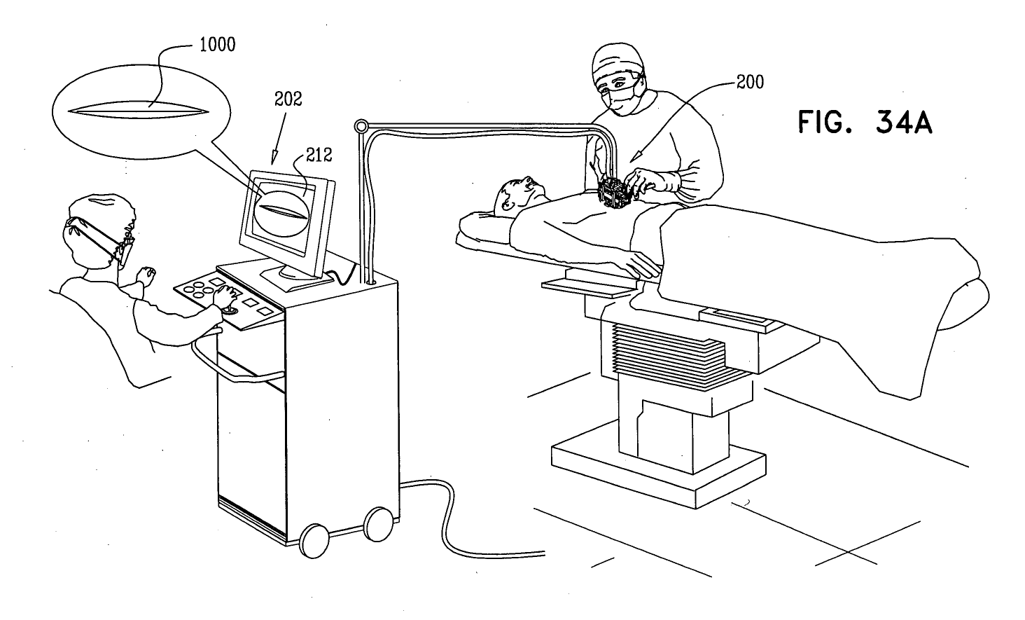

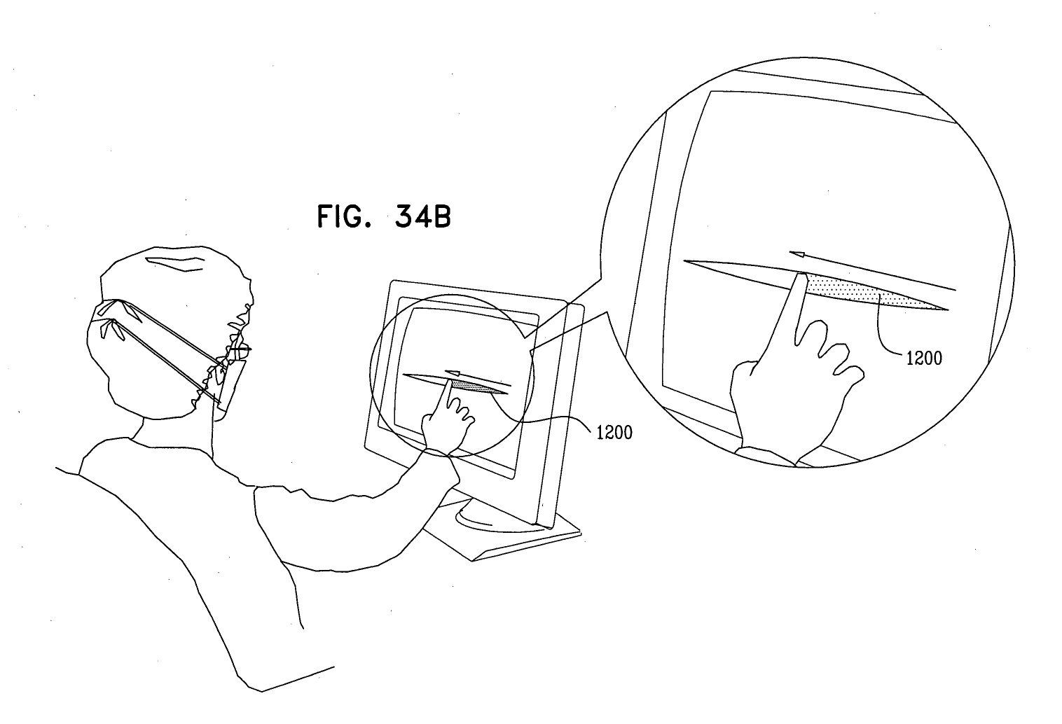

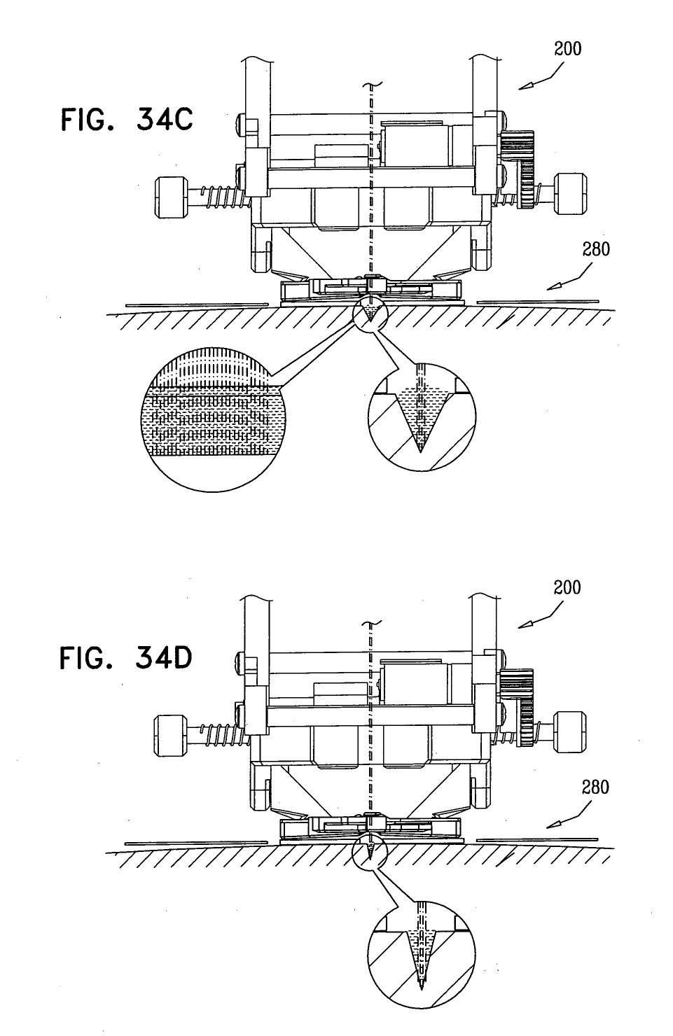

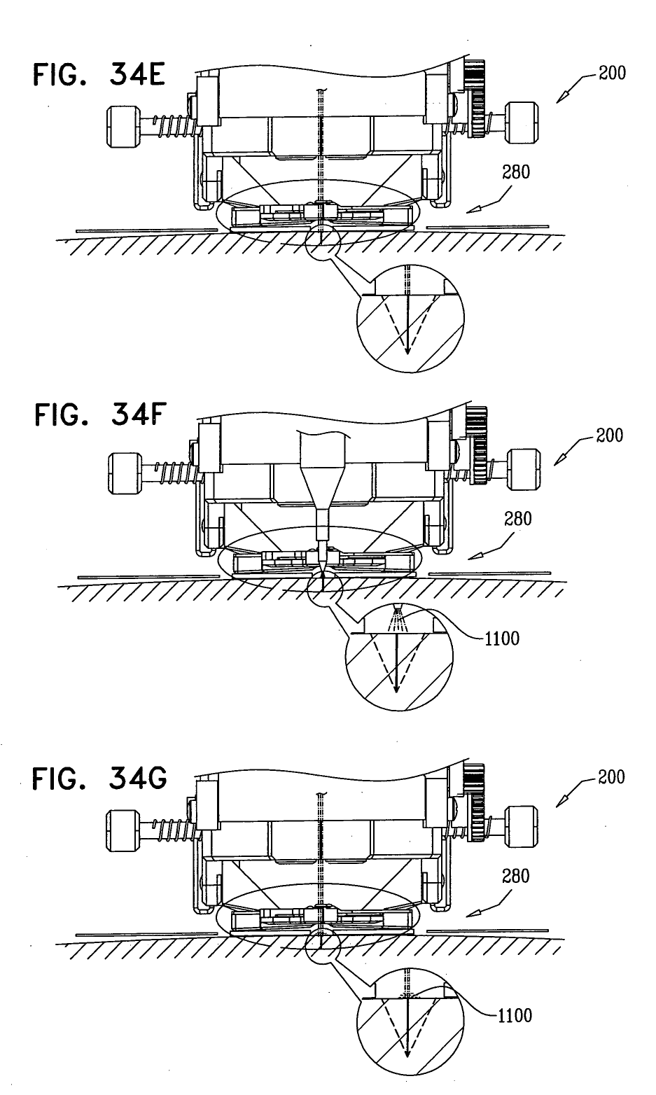

Reference is now made to FIGS. 34A, 34B, 34C, 34D, 34E, 34F and 34G, which are each a simplified pictorial illustration of a still further stage in the operation of the system of FIGS. 1A, 1B and 3-26C wherein energy is applied to the cut by the energy directing subassembly (FIGS. 26A-26C) in coordination with operation of the clamp manipulation subassembly (FIGS. 21A-21D).

FIG. 34A shows an operator, employing operator interface screen 212 which displays a video feed from video camera 992 (FIGS. 26A-26C) forming part of the patient-mounted portion 200, visualizing open cut 1000. The operator may employ touch screen functionality associated with interface screen 212, or any other suitable graphic input functionality, to draw an energy application swath 1200 over the cut 1000, as shown in FIG. 34B. Alternatively, energy application mapping may be carried out automatically by software in the non patient-mounted portion 202. It is appreciated that the system of the present invention is adapted for use with any suitable configuration of cut, which need not be straight and may contain multiple curves in three dimensions.

FIG. 34C shows a visualization of initial scanning application of energy within the energy application swath 1200 over the open cut 1000 by the energy directing subassembly 260 (FIGS. 26A-26C), typically including optical scanner 984, which receives energy, such as laser energy, via an optical fiber or other light transmitting mechanism.

It is a particular feature of the present invention that as application of energy to the open cut 1000 takes place, the clamp manipulation subassembly 256 (FIGS. 21A-21D) operates clamp 280 in engagement with the patient tissue surrounding the cut 1000 to gradually close the cut. This gradual closing of the cut together, during or intermittently with application of energy to the cut is illustrated in FIGS. 34D and 34E.

Optionally, a further layer of bonding enhancement material 1100 may applied to the closed cut 1000, as shown in FIG. 34F, followed by further application of energy as illustrated in FIG. 34G.

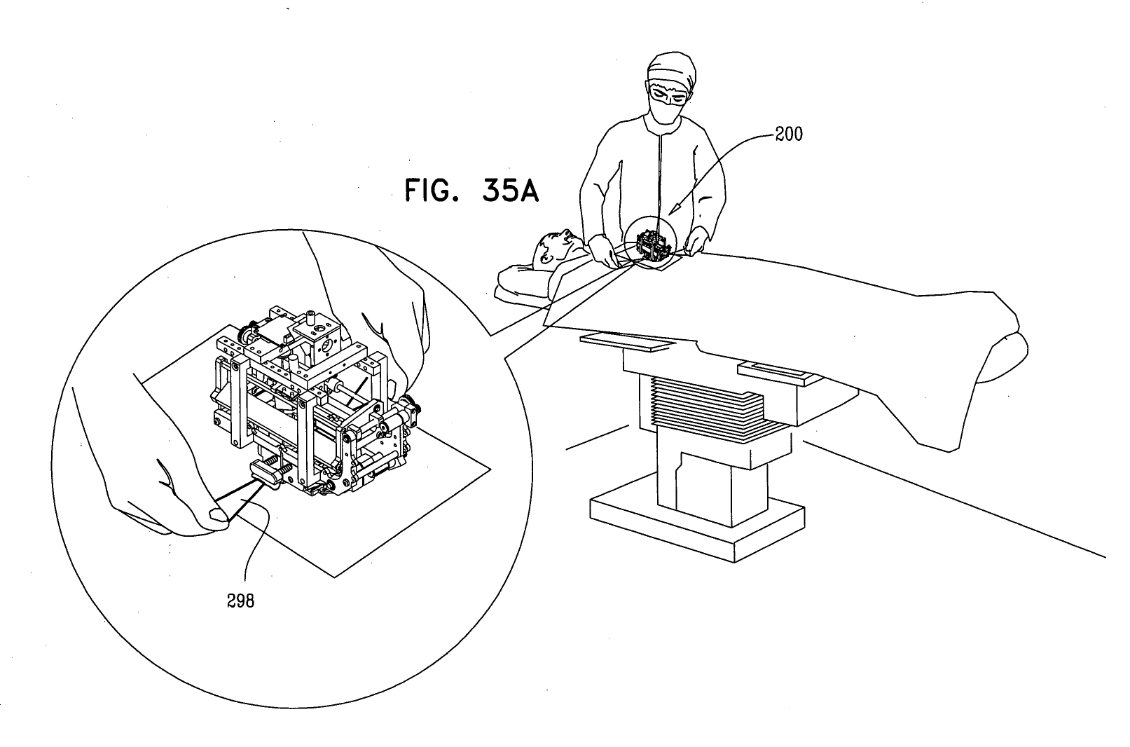

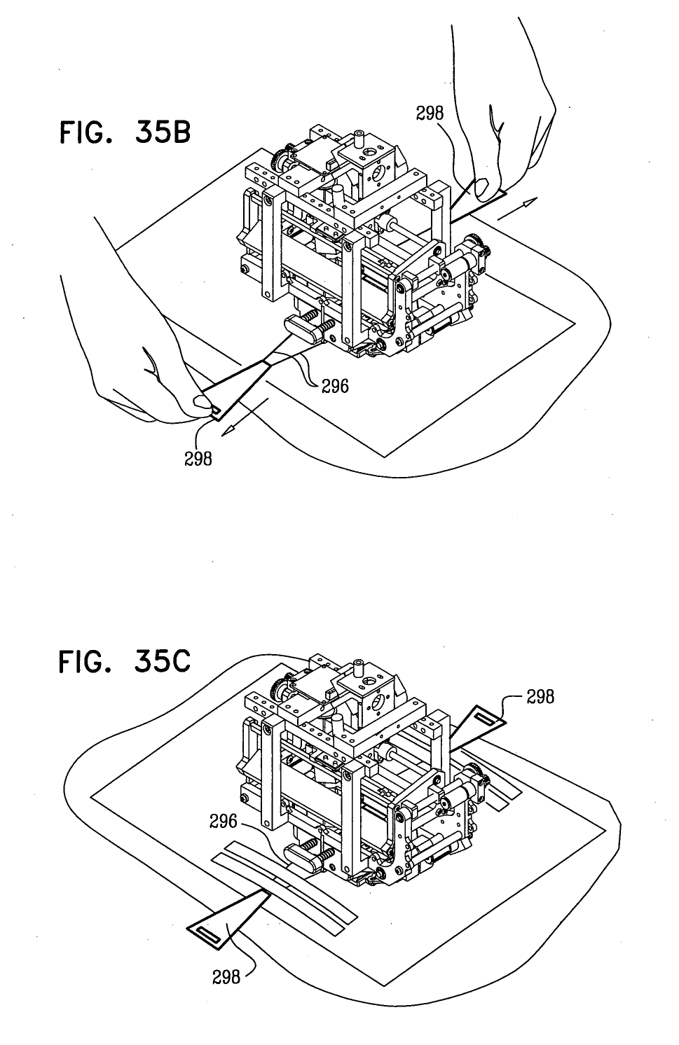

Reference is now made to FIGS. 35A, 35B and 35C, which illustrate an additional stage in the operation of the system of FIGS. 1A, 1B and 3-26C wherein following application of energy by the energy directing subassembly (FIGS. 26A-26C) in coordination with operation of the clamp manipulation subassembly (FIGS. 21A-21D), an operator secures the cut in a bonded orientation by use of the clamp assembly (FIG. 6).

FIG. 35A shows an operator whose hands engage pull tabs 298 (FIG. 6) of clamp assembly 280 (FIG. 6), while the clamp portion 282 is held in position by the clamp manipulation assembly 256 (FIGS. 21A-21D). FIG. 35B shows the operator pulling tabs 298, thus tensioning elongate elements 296 and thereby securing the mutual orientations of bandage portions 288 (FIG. 6), thus securing the cut 1000 in a desired closed orientation. FIG. 35C shows retaining elongate elements 296 in their tensioned orientation by taping them onto either or both of bandage portions 288 and the patient tissue.

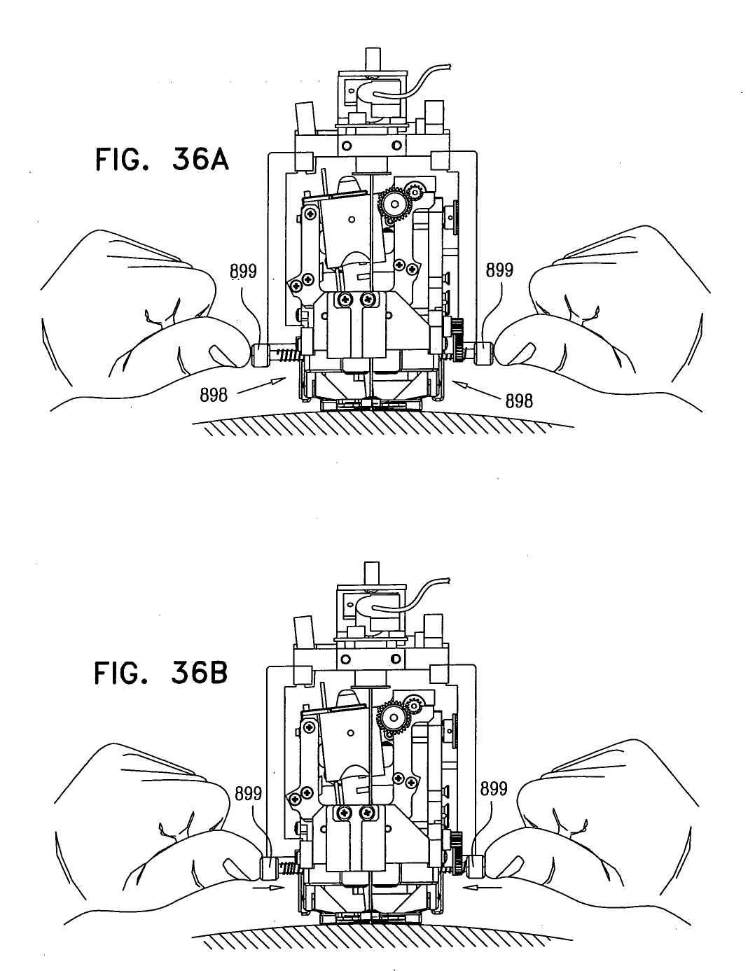

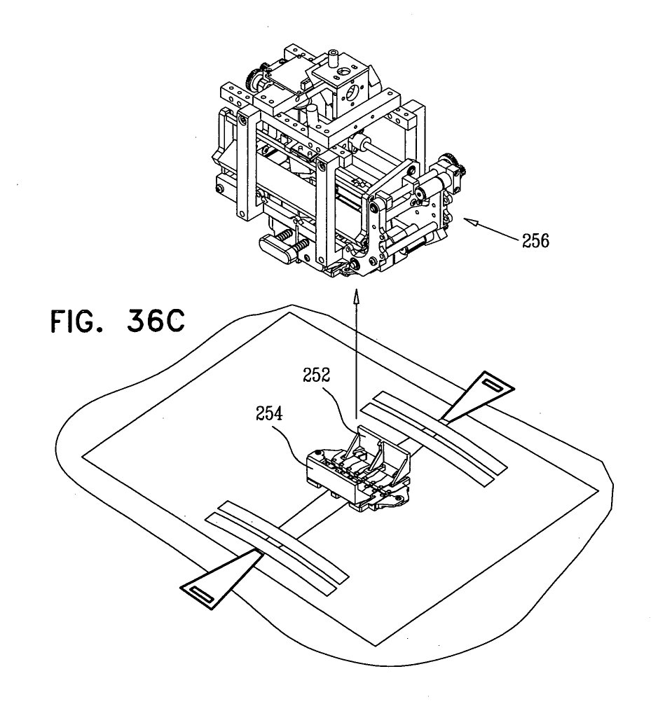

Reference is now made to FIGS. 36A, 36B and 36C, which are each a simplified illustration of a further additional stage in the operation of the system of FIGS. 1A, 1B and 3-21D and 24A-26C wherein the clamp engagement elements 252 and 254 (FIGS. 20A and 20B) are released from the clamp manipulation subassembly 256 (FIGS. 21A-21D) and disengaged from the clamp assembly.

FIG. 36A shows an operator about to press finger engagement portions 899 forming part of corresponding spring-loaded, manually operable engagement assembly 898. Pressing of finger engagement portions 899, shown in FIG. 36B, causes engagement plates 900, having formed thereon protrusions 902 (FIGS. 21A-21D) which engage respective apertures 834 (FIGS. 20A and 20B) formed in clamp engagement elements 252 and 254, to be displaced against the urging of springs 904 away from respective displacers 862 and 864. This allows disengagement of clamp engagement elements 252 and 254 from respective displacers 862 and 864 and engagement plates 900.

FIG. 36C shows the clamp manipulation subassembly 256 (FIGS. 21A-21D) having clamp engagement elements 252 and 254 disengaged therefrom.

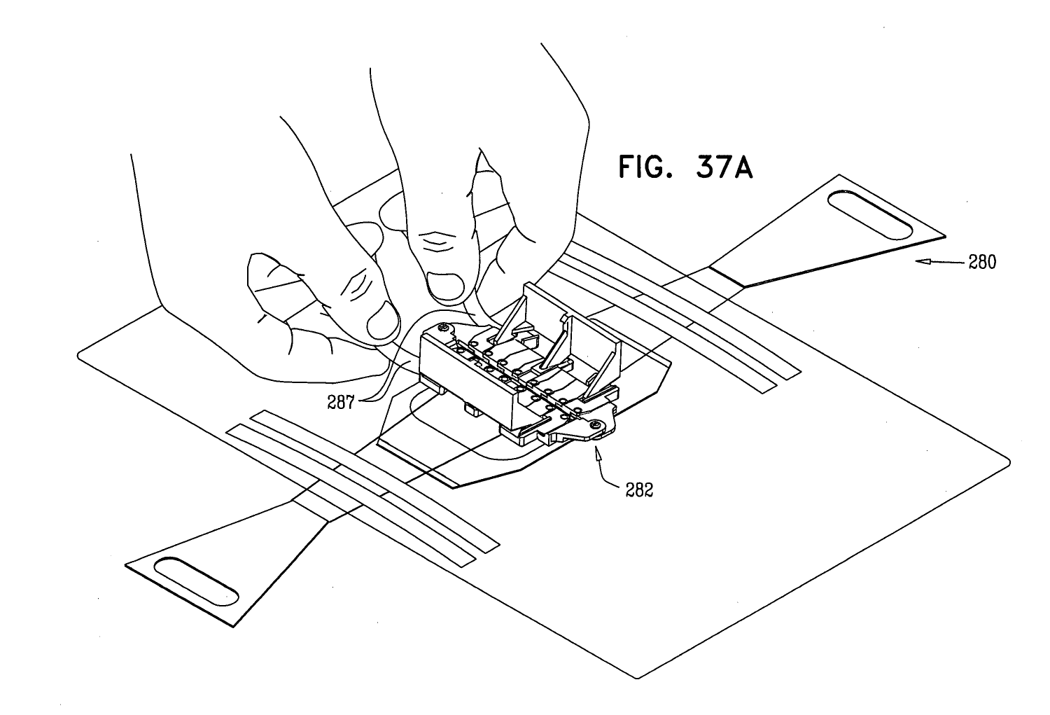

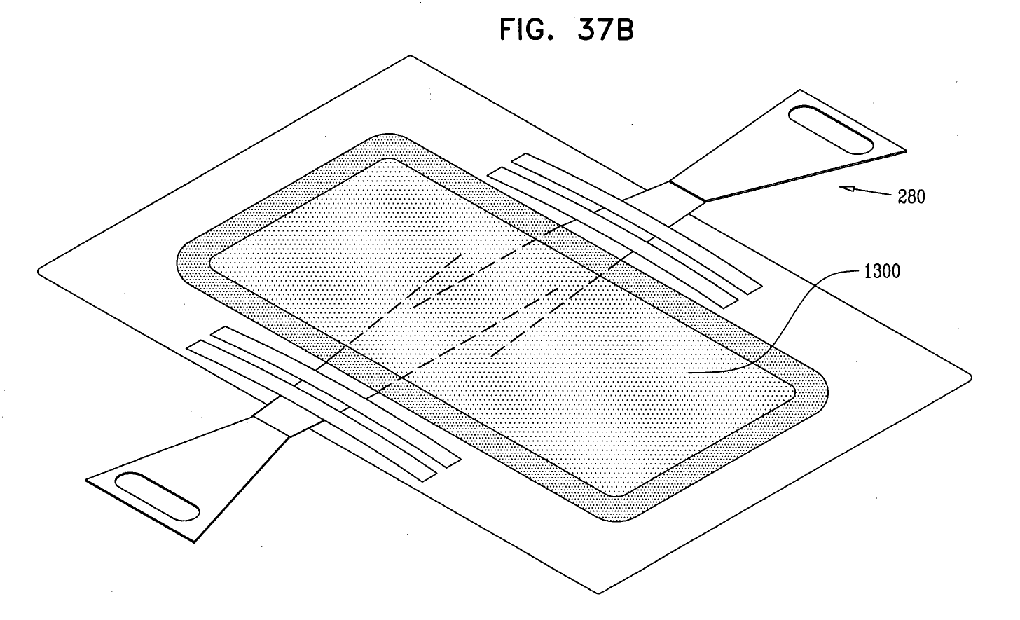

Reference is now made to FIGS. 37A and 37B, which illustrate a final stage in the operation of the system of FIGS. 1A, 1B and 3-21D and 24A-26C wherein the clamp portion is separated from the remainder of the clamp assembly and an auxiliary bandage is placed over the cut and the remainder of the clamp assembly.

FIG. 37A shows an operator pulling tabs 287 of flexible substrates 286 (FIG. 6), away from the cut 1000 in a direction generally perpendicular to the plane of the cut, thus disengaging the clamp portion 282 from the remainder of the clamp assembly 280. FIG. 37B shows application of an auxiliary bandage 1300 over the closed cut 1000 and the remainder of the clamp assembly 280, which retains the cut 1000 in a closed orientation until sufficient healing has taken place.

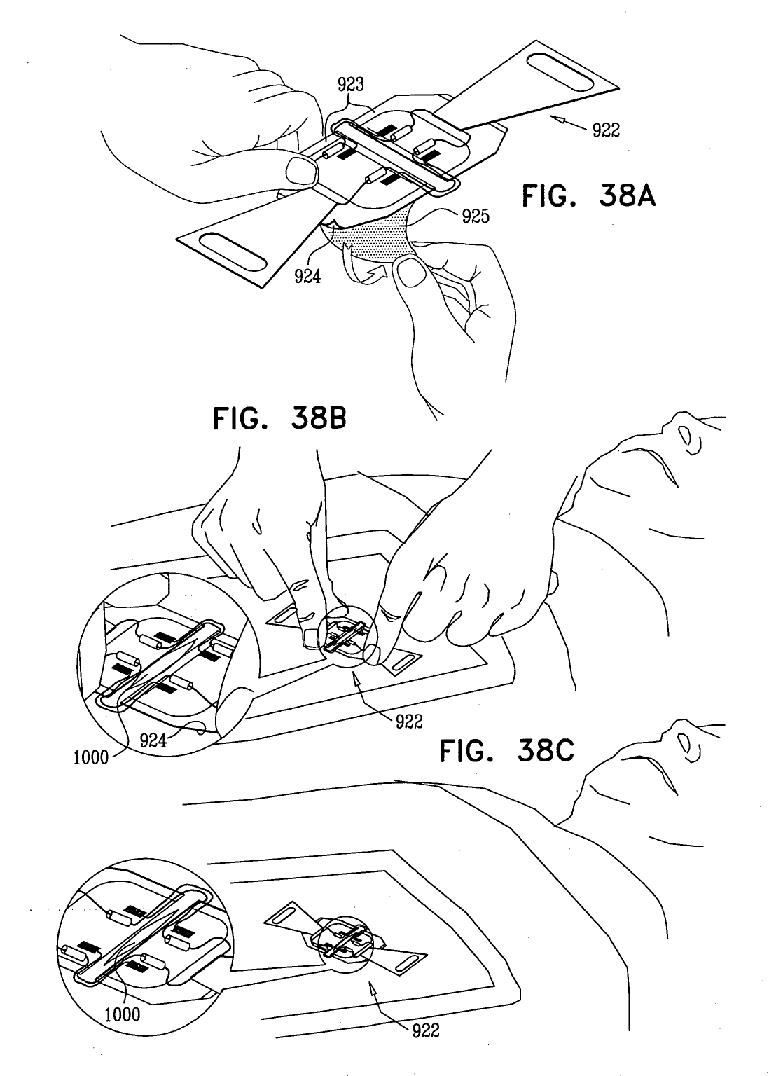

Reference is now made to FIGS. 38A, 38B and 38C, which are each a simplified pictorial illustration of a stage in the operation of the system of FIGS. 1A, 1B and 3-5 and 22A-26C wherein the tissue manipulation subassembly 905 (FIGS. 22A & 22B) is employed instead of the clamp manipulation subassembly 256. FIGS. 38A-38C illustrate a preferred, but not essential, scenario wherein a bandage assembly 922 (FIG. 23) is mounted onto a patient in propinquity to a cut to be bonded.

FIG. 38A shows removal of release layers 925 from adhesive surfaces 924 of side bandage portions 923. FIG. 38B shows placement of the bandage assembly 922 (FIG. 23) over a cut 1000 with adhesive surfaces 924 being pressed into adhesive engagement with the patient's skin adjacent cut 1000. FIG. 38C shows the bandage assembly 922 (FIG. 23) retained in place over cut 1000 by this adhesive engagement.

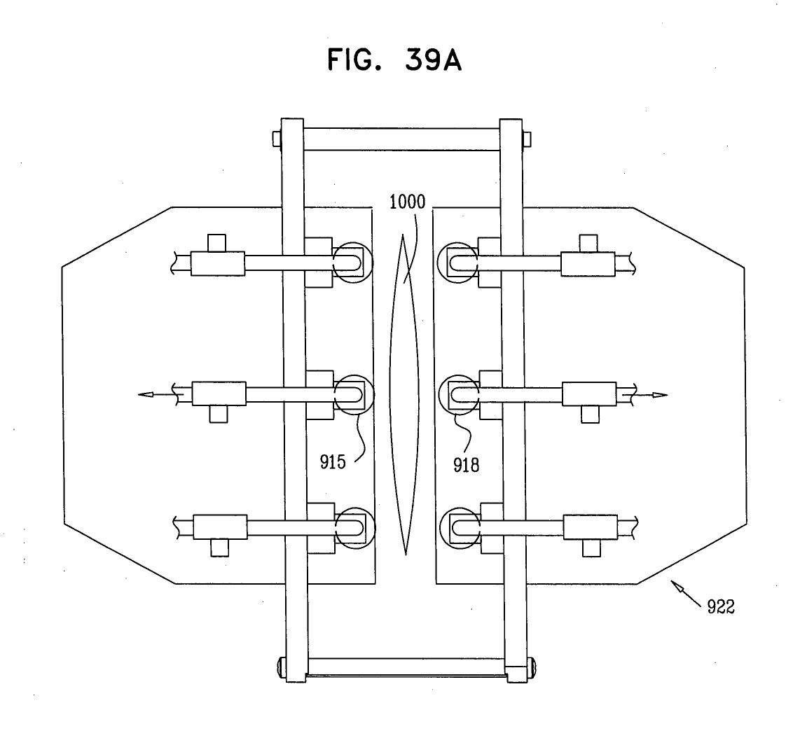

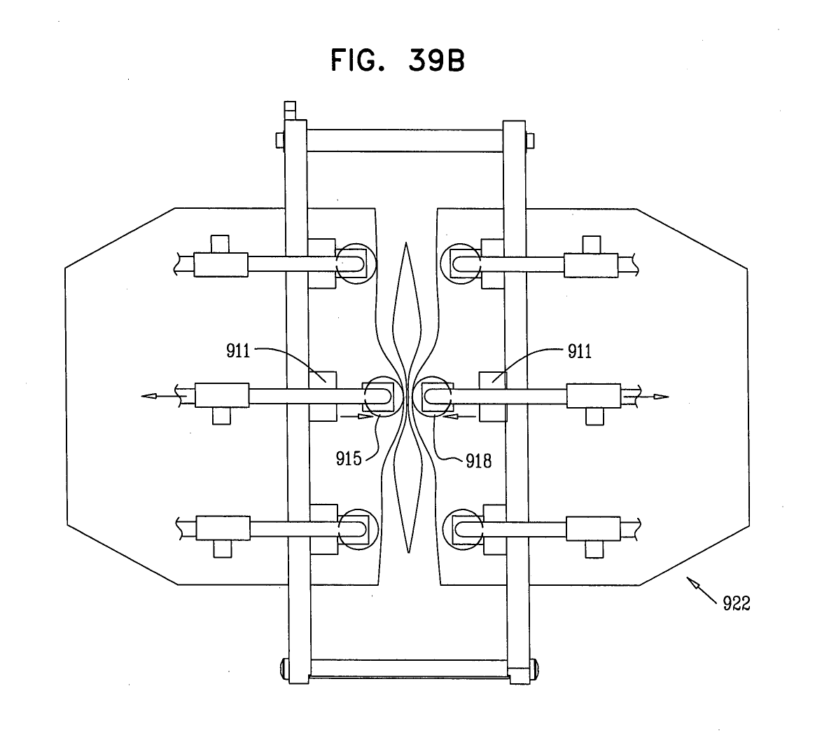

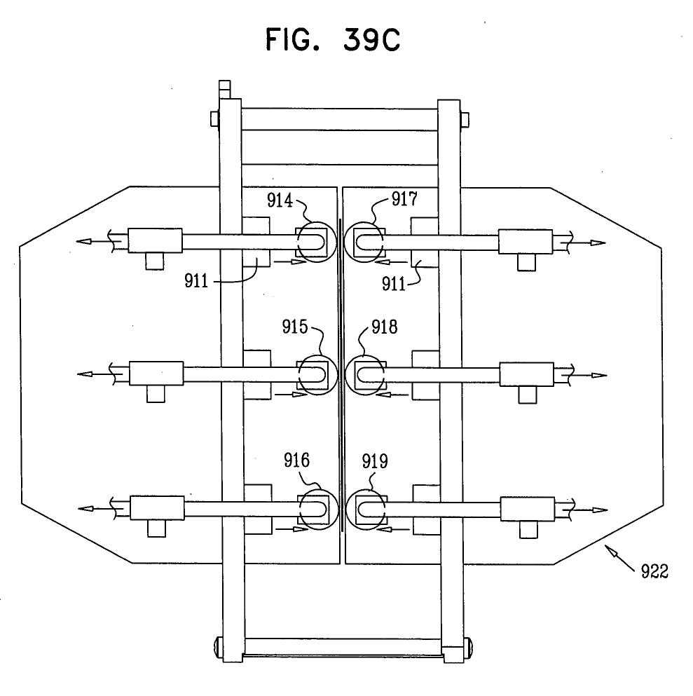

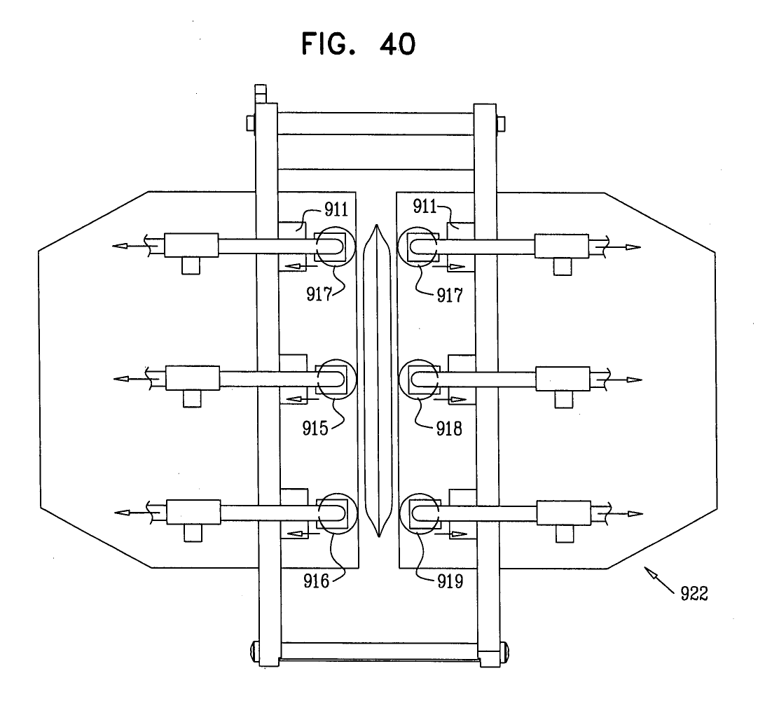

Reference is now made to FIGS. 39A, 39B and 39C, which are each a simplified pictorial illustration of a further stage in the operation of the system of FIGS. 1A, 1B and 3-5 and 22A-26C, wherein the tissue manipulation subassembly 905 (FIGS. 22A & 22B) is employed instead of the clamp manipulation subassembly 256 for providing initial closing of cut 1000, prior to application of energy or of bonding enhancement material to the cut 1000.

FIG. 39A shows placement of the patient mounted portion 200 (FIGS. 4A-5) of the system onto bandage assembly 922 by an operator. The operator orients the patient mounted portion 200 such that initially only the centrally located tissue engagement heads, here vacuum cups 915 and 918, are located in non-slidable vacuum engagement with the bandage assembly 922, generally at the center of the cut 1000 and are slightly spaced from respective opposite edges thereof.

FIG. 39B shows initial closing operation of the tissue manipulation subassembly 905 (FIGS. 22A & 22B) controlled by the operator, wherein vacuum cups 915 and 918 are brought closer to each other, such that cut 1000 is closed at a center location thereof adjacent vacuum cups 915 and 918. This initial closing operation is produced by corresponding relative displacement of individually controllable linear motors 911 (FIGS. 22A & 22B)

FIG. 39C shows further closing operation of tissue manipulation subassembly 905 (FIGS. 22A & 22B) controlled by the operator, wherein vacuum cups 914 & 916 and 917 & 919, presently in non-slidable vacuum engagement with the bandage assembly 922, are brought closer to each other such that cut 1000 is closed along substantially its entire length. This further closing operation is produced by corresponding relative displacement of individually controllable linear motors 911 (FIGS. 22A & 22B).

It is appreciated that the orientation the edges of the skin along cut 1000 is generally along a straight line, defined by the longitudinal axis of the cut 1000, as distinguished from bowed orientations seen in FIGS. 39A and 39B, thus providing an esthetically optimized closure. This is preferably achieved by individual actuation of linear motors 911.

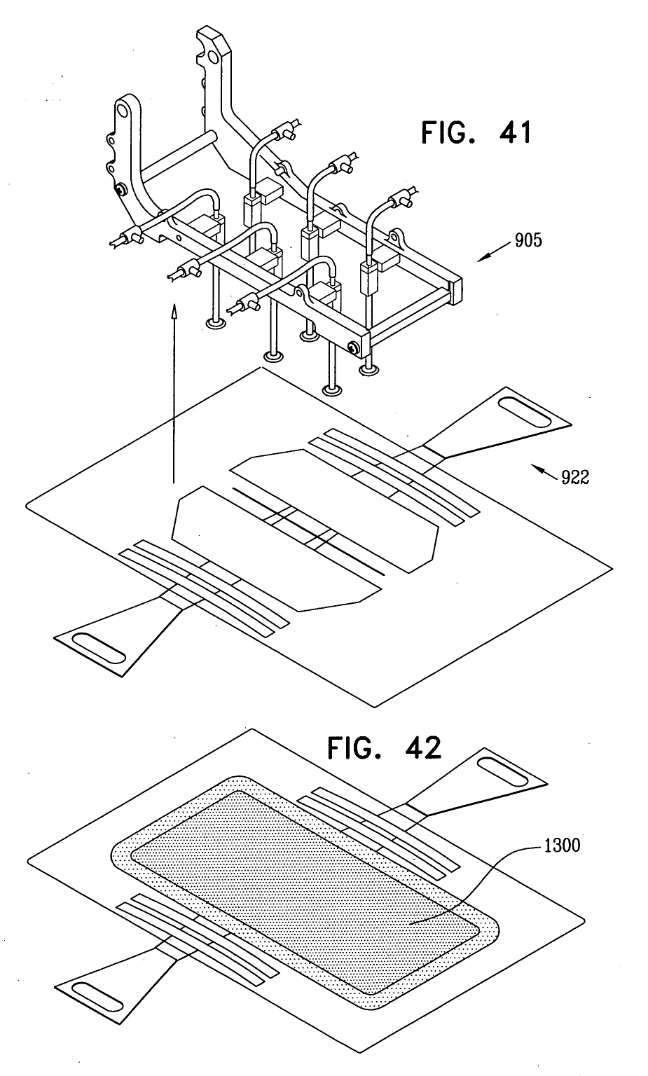

Reference is now made to FIG. 40, which shows limited axial opening of the cut 1000 by opening operation of tissue manipulation subassembly 905 (FIGS. 22A & 22B) controlled by the operator, wherein vacuum cups 914-916 and 917-919, all presently in non-slidable vacuum engagement with the bandage assembly 922, are moved away from each other, along an axis which is perpendicular to the longitudinal axis of the cut 1000. This limited opening operation is produced by corresponding relative displacement of suitable ones of linear motors 911.

Reference is now made to FIGS. 31A & 31B, 33A-34G and 35A-35C, described above, which are equally applicable to the embodiment of FIGS. 1A, 1B and 3-5 and 22A-26C, wherein the tissue manipulation subassembly 905 (FIGS. 22A & 22B) is employed instead of the clamp manipulation subassembly 256.

It is a particular feature of the present invention that as application of energy to the open cut 1000 takes place, the tissue engagement subassembly 905 (FIGS. 22A & 22B) operates in engagement with the patient tissue surrounding the cut 1000 to gradually close the cut. This gradual closing of the cut together, during or intermittently with application of energy to the cut is illustrated in FIGS. 34D and 34E.

Optionally, a further layer of bonding enhancement material may applied to the closed cut 1000, as shown in FIG. 34F, followed by further application of energy as illustrated in FIG. 34G.

Similar to the embodiment shown in FIGS. 35B and 35C, an operator pulls pull tabs 929 (FIG. 23) of bandage assembly 922, thus tensioning elongate elements 928 (FIG. 23) and thereby securing the mutual orientations of bandage portions 923 (FIG. 23), thus securing the cut 1000 in a desired closed orientation. Elongate elements 928 may be retained in their tensioned orientation by taping them onto either or both of bandage portions 923 and the patient tissue.

Reference is now made to FIG. 41, which is a simplified illustration of a further additional stage in the operation of the system of FIGS. 1A, 1B and 3-5 and 22A-26C, wherein the tissue manipulation subassembly 905 (FIGS. 22A & 22B) is employed instead of the clamp manipulation subassembly 256.

FIG. 41 shows an operator releasing vacuum engagement between vacuum cups 914-919 and the patient and disengaging the patient mounted portion 200 (FIGS. 4A-5) from the patient.

Reference is now made to FIG. 42, which illustrates a final stage in the operation of the system of FIGS. 1A, 1B and 3-5 and 22A-26C wherein the tissue manipulation subassembly 905 (FIGS. 22A & 22B) is employed instead of the clamp manipulation subassembly 256, wherein an auxiliary bandage 1300 is placed over the cut, thereby retaining the cut 1000 in a closed orientation until sufficient healing has taken place.

It will be appreciated by persons skilled in the art that the present invention is not limited to what has been particularly shown and described hereinabove. Rather the scope of the present invention includes both combinations and subcombinations of various features described hereinabove as well as modifications thereof which would occur to a person of skill in the art upon reading the foregoing description and which are not in the prior art.

1. A system for bonding living tissue comprising:
first tissue displacer functionality operative initially to cause displacement of edges of tissue into a mutual touching relationship along a seam prior to bonding thereof; and
second tissue displacer functionality operative following operation of said first tissue displacer functionality to cause displacement of said edges into a mutual non-touching relationship wherein said edges are in mutual propinquity.

2. A system for bonding living tissue according to claim 1 and also comprising:
tissue bonding functionality operative following operation of said first and second displacer functionalities to apply energy to said edges of said tissue; and
third tissue displacer functionality operative to cause displacement of said edges of said tissue into said mutual touching relationship along said seam for bonding thereof.

3. A system for bonding living tissue according to claim 2 and also comprising:
a tissue bonding enhancement material dispenser operative following operation of said second tissue displacer functionality and at least partially prior to said tissue bonding functionality to apply a tissue bonding enhancement material to said edges when said edges are in said mutual non-touching relationship wherein said edges are in mutual propinquity.

4. A system for bonding living tissue according to claim 1 and wherein said first and second tissue displacer functionalities employ a tissue displacer assembly comprising:
at least first and second tissue engagement elements arranged for removable engagement with tissue adjacent opposite facing edges thereof and relative displacement thereof, thereby to bring said opposite facing edges into mutual propinquity, at least one of said at least first and second tissue engagement elements being transparent to radiant energy at least at a region thereof overlying said opposite facing edges.

5. A system for bonding living tissue according to claim 1 and wherein said first and second tissue displacer functionalities employ a tissue displacer assembly comprising:
at least first and second tissue engagement elements arranged for removable engagement with tissue adjacent opposite facing edges thereof; and
at least first and second linearly displaceable elements operatively engageable with said first and second tissue engagement elements for providing relative displacement of said at least first and second tissue engagement elements, wherein operative engagement of said at least first and second linearly displaceable elements with said at least first and second tissue engagement elements includes at least the following modes of operation:
a first closing mode of operation wherein generally parallel displacement of said first and second linearly displaceable elements towards each other produces generally non-parallel displacement of said at least first and second tissue engagement elements towards each other;
a second opening mode of operation wherein generally parallel displacement of said first and second linearly displaceable elements away from each other produces generally parallel displacement of said at least first and second tissue engagement elements away from each other; and
a third closing mode of operation wherein linear displacement of said first and second linearly displaceable elements towards each other produces generally parallel displacement of said at least first and second tissue engagement elements towards each other.

6. A system for bonding living tissue according to claim 1 and wherein said first and second tissue displacer functionalities employ a tissue displacer assembly comprising:
at least first and second tissue engagement elements arranged for removable engagement with tissue adjacent opposite facing edges thereof for providing relative displacement of said opposite facing edges into and out of mutual propinquity.

7. A system for bonding living tissue according to claim 1 and wherein said first and second tissue displacer functionalities employ a tissue displacer assembly comprising:
at least first and second tissue engagement elements arranged for removable engagement with tissue adjacent opposite facing edges thereof and relative displacement thereof, thereby to bring said opposite facing edges into desired mutual propinquity; and
a bonding enhancement material dispenser mounted with respect to at least one of said at least first and second tissue engagement elements for dispensing tissue bonding enhancement material onto said opposite facing edges when in said desired mutual propinquity.

8. A system for bonding living tissue according to claim 1 and wherein said first and second tissue displacer functionalities employ a tissue displacer assembly comprising:
at least first and second tissue engagement elements arranged for removable engagement with tissue adjacent opposite facing edges thereof; and
at least first and second displaceable elements operatively engageable with said first and second tissue engagement elements for providing relative displacement of said at least first and second tissue engagement elements into and out of propinquity, such that said opposite facing edges of said tissue are mutually displaced into and out of touching engagement; and
at least one retainer operative, independently of the first and second displaceable elements, for retaining said at least first and second tissue engagement elements in propinquity, such that said opposite facing edges of said tissue are in touching engagement.

9. A system for bonding living tissue according to claim 3 and wherein said tissue bonding enhancement material dispenser is at least partially integrated with a tissue displacer assembly comprising:
at least first and second tissue engagement elements arranged for removable engagement with tissue adjacent opposite facing edges thereof and relative displacement thereof, thereby to bring said opposite facing edges into mutual propinquity, at least one of said at least first and second tissue engagement elements being transparent to radiant energy at least at a region thereof overlying said opposite facing edges.

10. A system for bonding living tissue according to claim 3 and wherein said tissue bonding enhancement material dispenser comprises a gas driven sprayer for applying said tissue bonding enhancement material onto said edges.

11. A system for bonding living tissue comprising:
tissue bonding functionality operative to apply energy to edges of tissue and to cause displacement of said edges of said tissue into mutual touching relationship along a seam for bonding thereof.

12. A system for bonding living tissue according to claim 11 and also comprising a tissue bonding enhancement material dispenser operative at least partially prior to operation of said tissue bonding functionality to apply a tissue bonding enhancement material to said edges when said edges are in said mutual non-touching relationship wherein said edges are in mutual propinquity.

13. A system for bonding living tissue according to claim 11 and also comprising:
tissue displacer functionality operative prior to operation of said tissue bonding functionality to cause displacement of said edges into a mutual non-touching relationship wherein said edges are in mutual propinquity.

14. A system for bonding living tissue according to claim 11 and also comprising:
first tissue displacer functionality operative initially to cause displacement of edges of tissue into a mutual touching relationship along a seam prior to bonding thereof; and
second tissue displacer functionality operative following operation of said first tissue displacer functionality to cause displacement of said edges into a mutual non-touching relationship wherein said edges are in mutual propinquity.

15. A system for bonding living tissue according to claim 11 and wherein said tissue bonding functionality is operative initially to apply energy to relatively more interior portions of said edges of said tissue and to cause displacement of said relatively more interior portions of said edges of said tissue into said mutual touching relationship along said seam for bonding thereof and thereafter to apply energy to relatively less interior portions of said edges of said tissue and to cause displacement of said relatively less interior portions of said edges of said tissue into said mutual touching relationship along said seam for bonding thereof.

16. A system for bonding living tissue according to claim 15 and wherein said tissue bonding functionality is operative sequentially initially to apply energy to relatively more interior portions of said edges of said tissue and to cause displacement of said relatively more interior portions of said edges of said tissue into said mutual touching relationship along said seam for bonding thereof and thereafter sequentially to apply energy to increasingly less interior portions of said edges of said tissue and to cause displacement of said increasingly less interior portions of said edges of said tissue into said mutual touching relationship along said seam for bonding thereof.

17. A system for bonding living tissue according to claim 13 and wherein said tissue displacer functionality employs a tissue displacer assembly comprising:
at least first and second tissue engagement elements arranged for removable engagement with tissue adjacent opposite facing edges thereof and relative displacement thereof, thereby to bring said opposite facing edges into mutual propinquity, at least one of said at least first and second tissue engagement elements being transparent to radiant energy at least at a region thereof overlying said opposite facing edges.

18. A system for bonding living tissue according to claim 14 and wherein said first and second tissue displacer functionalities employ a tissue displacer assembly comprising:
at least first and second tissue engagement elements arranged for removable engagement with tissue adjacent opposite facing edges thereof; and
at least first and second linearly displaceable elements operatively engageable with said first and second tissue engagement elements for providing relative displacement of said at least first and second tissue engagement elements, wherein operative engagement of said at least first and second linearly displaceable elements with said at least first and second tissue engagement elements includes at least the following modes of operation:
a first closing mode of operation wherein generally parallel displacement of said first and second linearly displaceable elements towards each other produces generally non-parallel displacement of said at least first and second tissue engagement elements towards each other;
a second opening mode of operation wherein generally parallel displacement of said first and second linearly displaceable elements away from each other produces generally parallel displacement of said at least first and second tissue engagement elements away from each other; and
a third closing mode of operation wherein linear displacement of said first and second linearly displaceable elements towards each other produces generally parallel displacement of said at least first and second tissue engagement elements towards each other.

19. A system for bonding living tissue according to claim 14 and wherein said first and second tissue displacer functionalities employ a tissue displacer assembly comprising:
at least first and second tissue engagement elements arranged for removable engagement with tissue adjacent opposite facing edges thereof for providing relative displacement of said opposite facing edges into and out of mutual propinquity.

20. A system for bonding living tissue according to claim 14 and wherein said first and second tissue displacer functionalities employ a tissue displacer assembly comprising:
at least first and second tissue engagement elements arranged for removable engagement with tissue adjacent edges thereof and relative displacement thereof, thereby to bring said edges into desired mutual propinquity; and
a bonding enhancement material dispenser mounted with respect to at least one of said at least first and second tissue engagement elements for dispensing tissue bonding enhancement material onto said edges when in said desired mutual propinquity.

21. A system for bonding living tissue comprising:
tissue bonding functionality operative to apply energy to edges of tissue for bonding thereof along a seam and including a computer-controlled energy impingement location displacer for displacement of an impingement location of said energy along said seam.

22. A system for bonding living tissue according to claim 21 and wherein said computer-controlled energy impingement location displacer comprises a displacer that is capable of movement in at least two mutually perpendicular directions in a plane.

23. A system for bonding living tissue according to claim 21 and wherein said computer-controlled energy impingement location displacer comprises a displacer that is capable of movement in at least three mutually perpendicular directions.

24. A system for bonding living tissue according to claim 21 and wherein said computer-controlled energy impingement location displacer includes a seam designator operative to designate said seam in a frame of reference and is operative to displace said impingement location of said energy along said seam, designated by said seam designator.

25.-26. (canceled)

27. A system for bonding living tissue according to claim 21 and wherein said tissue bonding functionality also comprises an optical waveguide carrying energy from a laser and wherein said computer-controlled energy impingement location displacer displaces at least an end of said optical waveguide.

28. A system for bonding living tissue according to claim 21 and also comprising a computer-controlled tissue displacer operative to cause displacement of said edges of said tissue into mutual touching relationship along said seam for bonding thereof in coordination with operation of said computer-controlled energy impingement location displacer, whereby application of energy to said edges of said tissue at a given location is quickly followed by displacement of said edges of said tissue at said location into mutual touching relationship.

29. A system for bonding living tissue according to claim 28 and wherein the operation of said computer-controlled tissue displacer and of said computer-controlled energy impingement location displacer are coordinated such that application of energy to said edges of said tissue at a given location followed by displacement of said edges of said tissue at said location into mutual touching relationship proceeds sequentially along said seam.

30. A system for bonding living tissue according to claim 21 and wherein said computer-controlled energy impingement location displacer includes a displaceable optical pathway and also comprising a temperature sensor operative to sense temperature at said impingement location of said energy along said seam via at least part of said displaceable optical pathway.

31. A system for bonding living tissue according to claim 28 and wherein said computer-controlled tissue displacer includes a tissue tension force sensor for measuring the amount of tensile force applied to said edges of said tissue when displaced into said mutually touching relationship.

32. A system for bonding living tissue according to claim 31 and wherein said tissue tension force sensor is operative to provide an output indication of a measured tissue tension parameter.

33. A system for bonding living tissue according to claim 21 and also comprising:
first tissue displacer functionality operative initially to cause displacement of edges of tissue into a mutual touching relationship along a seam prior to bonding thereof; and
second tissue displacer functionality operative following operation of said first tissue displacer functionality to cause displacement of said edges into a mutual non-touching relationship wherein said edges are in mutual propinquity.

34. A system for bonding living tissue according to claim 33 and wherein said first and second tissue displacer functionalities employ a tissue displacer assembly comprising:
at least first and second tissue engagement elements arranged for removable engagement with tissue adjacent opposite facing edges thereof and relative displacement thereof, thereby to bring said opposite facing edges into mutual propinquity, at least one of said at least first and second tissue engagement elements being transparent to radiant energy at least at a region thereof overlying said opposite facing edges.

35. A system for bonding living tissue according to claim 33 and wherein said first and second tissue displacer functionalities employ a tissue displacer assembly comprising:
at least first and second tissue engagement elements arranged for removable engagement with tissue adjacent opposite facing edges thereof; and
at least first and second linearly displaceable elements operatively engageable with said first and second tissue engagement elements for providing relative displacement of said at least first and second tissue engagement elements, wherein operative engagement of said at least first and second linearly displaceable elements with said at least first and second tissue engagement elements includes at least the following modes of operation:
a first closing mode of operation wherein generally parallel displacement of said first and second linearly displaceable elements towards each other produces generally non-parallel displacement of said at least first and second tissue engagement elements towards each other;
a second opening mode of operation wherein generally parallel displacement of said first and second linearly displaceable elements away from each other produces generally parallel displacement of said at least first and second tissue engagement elements away from each other; and
a third closing mode of operation wherein linear displacement of said first and second linearly displaceable elements towards each other produces generally parallel displacement of said at least first and second tissue engagement elements towards each other.

36. A system for bonding living tissue according to claim 33 and wherein said first and second tissue displacer functionalities employ a tissue displacer assembly comprising:
at least first and second tissue engagement elements arranged for removable engagement with tissue adjacent opposite facing edges thereof for providing relative displacement of said opposite facing edges into and out of mutual propinquity.

37. A system for bonding living tissue according to claim 33 and wherein said first and second tissue displacer functionalities employ a tissue displacer assembly comprising:
at least first and second tissue engagement elements arranged for removable engagement with tissue adjacent opposite facing edges thereof and relative displacement thereof, thereby to bring said edges into desired mutual propinquity; and
a bonding enhancement material dispenser mounted with respect to at least one of said at least first and second tissue engagement elements for dispensing tissue bonding enhancement material onto said edges when in said desired mutual propinquity.

38. A system for bonding living tissue according to claim 33 and wherein said first and second tissue displacer functionalities employ a tissue displacer assembly comprising:
at least first and second tissue engagement elements arranged for removable engagement with tissue adjacent opposite facing edges thereof;
at least first and second displaceable elements operatively engageable with said first and second tissue engagement elements for providing relative displacement of said at least first and second tissue engagement elements into and out of propinquity, such that said opposite facing edges of said tissue are mutually displaced into and out of touching engagement; and
at least one retainer operative, independently of the first and second displaceable elements, for retaining said at least first and second tissue engagement elements in propinquity, such that said opposite facing edges of said tissue are in touching engagement.

39. A system for bonding living tissue according to claim 21 and also comprising a tissue bonding enhancement material dispenser.

40. A system for bonding living tissue according to claim 39 and wherein said tissue bonding enhancement material dispenser comprises a gas driven sprayer for applying tissue bonding enhancement material onto said edges.

41. A tissue displacer assembly comprising:
at least first and second tissue engagement elements arranged for removable engagement with tissue adjacent opposite facing edges thereof; and
at least first and second linearly displaceable elements operatively engageable with said first and second tissue engagement elements for providing relative displacement of said at least first and second tissue engagement elements, wherein operative engagement of said at least first and second linearly displaceable elements with said at least first and second tissue engagement elements includes at least the following modes of operation:
a first closing mode of operation wherein generally parallel displacement of said first and second linearly displaceable elements towards each other produces generally non-parallel displacement of said at least first and second tissue engagement elements towards each other;
a second opening mode of operation wherein generally parallel displacement of said first and second linearly displaceable elements away from each other produces generally parallel displacement of said at least first and second tissue engagement elements away from each other; and
a third closing mode of operation wherein linear displacement of said first and second linearly displaceable elements towards each other produces generally parallel displacement of said at least first and second tissue engagement elements towards each other.

42. A tissue displacer assembly according to claim 41 and wherein at least one of said at least first and second tissue engagement elements is transparent to radiant energy at least at a region thereof overlying said opposite facing edges.

43. A tissue displacer assembly according to claim 41 and also comprising at least one retainer operative, independently of the first and second linearly displaceable elements, for retaining said at least first and second tissue engagement elements in propinquity, such that said opposite facing edges of said tissue are in touching engagement.

44. A tissue manipulation assembly comprising:
a chassis;
a plurality of individually controllable motors;
a plurality of arms each at least partially positioned by one of said plurality of motors; and
a tissue engagement head mounted onto each of said plurality of arms,
said tissue manipulation assembly having at least the following modes of operation:
a first closing mode of operation wherein generally non-parallel displacement of said first and second tissue engagement heads towards each other takes place; and
a second closing mode of operation wherein linear displacement of said first and second tissue engagement heads towards each other produces generally parallel displacement of said at least first and second tissue engagement elements towards each other.

45. A tissue manipulation assembly according to claim 44 and wherein said first closing mode of operation initially employs less than all of said tissue engagement heads.

46. A tissue manipulation assembly according to claim 44 and wherein said tissue engagement head comprises a vacuum engagement head arranged for selectable engagement with tissue adjacent a cut.

47. A tissue displacer assembly comprising:
at least first and second tissue engagement elements arranged for removable engagement with tissue adjacent opposite facing edges thereof and relative displacement thereof, thereby to bring said opposite facing edges into mutual propinquity, at least one of said at least first and second tissue engagement elements being transparent to radiant energy at least at a region thereof overlying said opposite facing edges.

48. A tissue displacer assembly according to claim 47 and also comprising at least one retainer operative, independently of the first and second displaceable elements, for retaining said at least first and second tissue engagement elements in propinquity, such that said opposite facing edges of said tissue are in touching engagement.

49. A tissue displacer assembly comprising:
at least first and second tissue engagement elements arranged for removable engagement with tissue adjacent opposite facing edges thereof for providing relative displacement of said opposite facing edges into and out of mutual propinquity.

50. A tissue displacer assembly according to claim 49 and wherein relative displacement of said at least first and second tissue engagement elements includes at least the following modes of operation:
a first closing mode of operation wherein generally parallel displacement of said first and second tissue engagement elements towards each other brings said opposite facing edges towards each other; and
a second opening mode of operation wherein generally parallel displacement of said first and second tissue engagement elements away from each other moves said opposite facing edges away from each other.

51. A tissue displacer assembly according to claim 49 and also comprising at least one retainer operative for retaining said at least first and second tissue engagement elements in propinquity, such that said opposite facing edges of said tissue are in touching engagement.

52. A tissue displacer assembly according to claim 51 and wherein said at least one retainer comprises a bandage assembly comprising:
a pair of side bandage portions, each having an adhesive surface for attachment to a patient's body and a release layer;
a plurality of conduits, fixed to each bandage portion; and
a plurality of elongate elements each extending through a corresponding one of the plurality of conduits, one end of each of the elongate elements extending through conduits on each side bandage portion being connected to a common pull-tab and an opposite end of each of the elongate elements extending through conduits on each side bandage portion being fixed to an opposite side bandage portion.

53. A tissue displacer assembly comprising:
at least first and second tissue engagement elements arranged for removable engagement with tissue adjacent opposite facing edges thereof and relative displacement thereof, thereby to bring said edges into desired mutual propinquity; and
a bonding enhancement material dispenser mounted with respect to at least one of said at least first and second tissue engagement elements for dispensing tissue bonding enhancement material onto said edges when in said desired mutual propinquity.

54. A tissue displacer assembly according to claim 53 and wherein said bonding enhancement material dispenser comprises a gas driven sprayer for applying said tissue bonding enhancement material onto said edges.

55. A tissue displacer assembly according to claim 53 and also comprising at least one retainer operative for retaining said at least first and second tissue engagement elements in propinquity, such that said edges of said tissue are in touching engagement

56. A tissue displacer assembly according to claim 55 and wherein said at least one retainer comprises a bandage assembly comprising:
a pair of side bandage portions, each having an adhesive surface for attachment to a patient's body and a release layer;
a plurality of conduits, fixed to each bandage portion; and
a plurality of elongate elements each extending through a corresponding one of the plurality of conduits, one end of each of the elongate elements extending through conduits on each side bandage portion being connected to a common pull-tab and an opposite end of each of the elongate elements extending through conduits on each side bandage portion being fixed to an opposite side bandage portion.

57. (canceled)

58. A tissue displacer assembly comprising:
at least first and second tissue engagement elements arranged for removable engagement with tissue adjacent opposite facing edges thereof;
at least first and second displaceable elements operatively engageable with said first and second tissue engagement elements for providing relative displacement of said at least first and second tissue engagement elements into and out of propinquity, such that said opposite facing edges of said tissue are mutually displaced into and out of touching engagement; and
at least one retainer operative, independently of the first and second displaceable elements, for retaining said at least first and second tissue engagement elements in propinquity, such that said opposite facing edges of said tissue are in touching engagement;
said at least one retainer comprising a bandage assembly comprising:
a pair of side bandage portions, each having an adhesive surface for attachment to a patient's body and a release layer;
a plurality of conduits, fixed to each bandage portion; and
a plurality of elongate elements each extending through a corresponding one of the plurality of conduits, one end of each of the elongate elements extending through conduits on each side bandage portion being connected to a common pull-tab and an opposite end of each of the elongate elements extending through conduits on each side bandage portion being fixed to an opposite side bandage portion.

59.-71. (canceled)
